(19) United States
(12) Patent Application Publication
(10) Pub. No.: US 2021/0196638 A1
(43) Pub. Date: Jul. 1, 2021

(54) ORAL DRUG DOSAGE FORMS HAVING A DESIRED PK PROFILE AND METHODS OF DESIGNING AND PRODUCING THEREOF

(71) Applicant: Triastek, Inc., Nanjing (CN)

(72) Inventors: Feihuang DENG, Nanjing (CN); Xin LIU, Nanjing (CN); Yu ZHENG, Nanjing (CN); Jie CHENG, Nanjing (CN); Senping CHENG, Nanjing (CN); Xiaoling LI, Dublin, CA (US)

(21) Appl. No.: 17/194,120

(22) Filed: Mar. 5, 2021

Related U.S. Application Data
(63) Continuation of application No. PCT/CN2020/100769, filed on Jul. 8, 2020.

(30) Foreign Application Priority Data
Sep. 6, 2019 (CN) PCT/CN2019/104722

(51) Int. Cl. A61K 9/20; B33Y 10/00; B33Y 80/00; B33Y 70/00; G16H 70/40; G16H 20/10

(52) U.S. Cl. CPC A61K 9/2095; B33Y 10/00; G16H 20/10; B33Y 70/00; G16H 70/40; B33Y 80/00

(57) ABSTRACT

The present disclosure, in some aspects, is directed to methods of designing an oral drug dosage form formulated and configured to have a desired pharmacokinetic profile. In other aspects, the present disclosure is directed to oral drug dosage forms having a desired pharmacokinetic profile, and methods of making, such as three-dimensional printing, such oral drug dosage forms.

### TECHNICAL FIELD

The present disclosure, in some aspects, is directed to methods of designing an oral drug dosage form formulated and configured to have a desired pharmacokinetic profile. In other aspects, the present disclosure is directed to oral drug dosage forms having a desired pharmacokinetic profile, and methods of making, such as three-dimensional printing, such oral drug dosage forms.

### BACKGROUND

The growing understanding of the mechanisms of drugs and reagents increasingly illustrates the importance of precision in vivo drug delivery to ensure optimized delivery in location, time, and amount, to achieve a desired pharmacokinetic profile for best use, efficacy, and safety of said drugs, drug candidates, and reagents. To achieve a desired pharmacokinetic profile, certain drugs and reagents may require, e.g., complex release profiles and/or administration dosing regimen. However, such demands required often run counter to manufacturing constraints and ensuring proper use and patient compliance via simplicity of administration, e.g., once-daily oral dosage forms or delivery systems. Additionally, the design of a drug dosage form capable of achieving a desired pharmacokinetic profile in an individual may not be readily obtained, even when based on such testing as in vitro release profile testing.

All references cited herein, including patent applications and publications, are incorporated by reference in their entirety.

### BRIEF SUMMARY

In some aspects, the present disclosure provides a method of designing an oral drug dosage form having a fixed amount of a drug and a desired composite pharmacokinetic (PK) profile in an individual, wherein the oral drug dosage form comprises a dosage unit comprising: a first modulated-release (MR1) portion comprising the drug; and a second modulated-release (MR2) portion comprising the drug, the method comprising: (a) obtaining a MR1 PK curve of a MR1 precursor drug dosage form comprising the MR1 portion in the individual: (b) obtaining a MR2 PK curve of a MR2 precursor drug dosage form comprising the MR2 portion in the individual; and (c) determining the relative amounts of the drug in the MR1 portion and the MR2 portion based on the MR1 PK curve and MR2 PK curve such that the MR1 portion and the MR2 portion when combined together produce the oral drug dosage form having the desired composite PK profile in the individual.

In another aspect, the present disclosure provides a method of designing an oral drug dosage form having a fixed amount of a drug and a desired composite pharmacokinetic (PK) profile in an individual, wherein the oral drug dosage form comprises a dosage unit comprising: a first modulated-release (MR1) portion comprising the drug; and a second modulated-release (MR2) portion comprising the drug, the method comprising: determining the relative amounts of the drug in the MR1 portion and the MR2 portion based on a MR1 PK curve of a MR1 precursor drug dosage form comprising the MR1 portion in the individual, and a MR2 PK curve of a MR2 precursor drug dosage form comprising the MR2 portion in the individual such that the MR1 portion and the MR2 portion when combined together produce the oral drug dosage form having the desired composite PK profile in the individual.

In some embodiments, the method further comprises obtaining the MR2 PK curve of the MR2 precursor drug dosage form comprising the MR2 portion in the individual.

In some embodiments, the method further comprises obtaining the MR1 PK curve of the MR1 precursor drug dosage form comprising the MR1 portion in the individual.

In some embodiments, the individual is a human. In some embodiments, the individual is selected from the group consisting of a dog, a rodent, a ferret, a pig, a guinea pig, a rabbit, and a non-human primate.

In some embodiments, the drug has linear pharmacokinetics.

In some embodiments, the MR1 portion is a MR1 layer. In some embodiments, the MR2 portion is a MR2 layer.

In some embodiments, the MR1 portion is an immediate-release (IR) portion, the IR portion having an immediate-release profile. In some embodiments, the MR2 portion is an extended-release (ER) portion, the ER portion having an extended-release profile.

In some embodiments, the MR1 portion is a first extended-release (ER) portion, the first ER portion having an extended-release profile, and the MR2 portion is a second extended-release (ER) portion, the second ER portion having an extended-release profile.

In some embodiments, the MR1 portion and the MR2 portion are stacked on top of each other. In some embodiments, the MR1 portion and the MR2 portion are positioned side-by-side with each other.

In some embodiments, the MR1 portion and MR2 portion are partially surrounded by a shell, and wherein the shell has a slower dissolution rate than the ER portion. In some embodiments, the shell is non-erodible.

In some embodiments, the MR1 portion has a top surface and a bottom surface, wherein the MR2 portion has a top surface and a bottom surface, and wherein the shell is in direct contact with both the MR1 portion and the MR2 portion and leaves one surface of the MR1 portion and/or one surface of the MR2 portion exposed.

In some embodiments, the MR1 portion is stacked on top of the MR2 portion, and wherein the shell leaves only the top surface of the MR1 portion exposed. In some embodiments, the bottom surface of the MR1 portion is in direct contact with the top surface of the MR2 portion.

In some embodiments, the dosage unit further comprises a third modulated-release (MR3) portion. In some embodiments, the MR3 portion is an IR portion, the IR portion having an immediate-release profile. In some embodiments, the MR3 portion is an ER portion, the ER portion having an extended-release profile. In some embodiments, the MR3 portion has a top surface and a bottom surface, wherein the MR2 portion is stacked on top of the MR3 portion, and wherein the shell leaves only the top surface of the MR1 portion exposed. In some embodiments, the bottom surface of the MR2 portion is in direct contact with the top surface of the MR3 portion.

In some embodiments, the MR2 portion is stacked on top of the MR1 portion, and wherein the shell leaves only the top surface of the MR2 portion exposed. In some embodiments, the bottom surface of the MR2 portion is in direct contact with the top surface of the MR1 portion.

In some embodiments, the MR1 portion is stacked on top of the MR2 portion, and wherein the shell leaves the top surface of the MR1 portion and the bottom surface of the MR2 portion exposed. In some embodiments, the bottom surface of the MR1 portion is in direct contact with the top surface of the MR2 portion. In some embodiments, the shell is between the MR1 portion and the MR2 portion. In some embodiments, the dosage unit further comprises an intermediate portion, wherein the intermediate portion is between the MR1 portion and the MR2 portion.

In some embodiments, the MR1 portion and the MR2 portion are positioned side-by-side with each other, wherein the shell leaves the top surface of both the MR1 portion and the MR2 portion exposed. In some embodiments, the MR1 portion has a side surface, wherein the MR2 portion has a side surface, and wherein the side surface of the MR1 portion is in directed contact with the side surface of the MR2 portion. In some embodiments, the shell separates the MR1 portion and the MR2 portion. In some embodiments, the dosage unit further comprises an intermediate portion, wherein the intermediate portion is between the MR1 portion and the MR2 portion.

In some embodiments, the oral drug dosage form comprises two dosage units. In some embodiments, the two dosage units are the same. In some embodiments, the two dosage units are different. In some embodiments, the two dosage units are stacked back-to-back. In some embodiments, the two dosage units are separated by the shell. In some embodiments, the two dosage units are separated by an intermediate portion.

In some embodiments, at least 80% the MR1 portion erodes within about 60 minutes following administration of the oral drug dosage form to the individual. In some embodiments, the MR1 portion comprises an erodible material.

In some embodiments, the MR2 portion comprises an erodible material, and wherein the drug contained in the MR2 portion is released from the oral drug dosage form over a period of at least about 5 hours.

In some embodiments, the desired composite PK profile is determined based on having an area under the curve (AUC) and Cₘₐₓ within an acceptable threshold of a reference PK curve of the drug. In some embodiments, the desired composite PK profile is further determined based on having a tₘₐₓ within an acceptable threshold of a reference PK curve of the drug.

In some embodiments, the method comprises selecting one or more parameters for the MR2 portion to obtain a desired release profile of the drug from the MR2 portion. In some embodiments, the one or more parameters is selected from the group consisting of: thickness, surface area, substrate erosion rate, and drug concentration in the MR2 portion.

In some embodiments, the method further comprises determining the MR1 PK curve and the MR2 PK curve and adjusting the relative amounts of the drug in the MR1 portion and the MR2 portion.

In some embodiments, the method further comprises determining a composite PK curve of the oral drug dosage form. In some embodiments, the method further comprises adjusting the relative amounts of the drug in the MR1 portion and the MR2 portion based on a comparison of the composite PK curve and the desired composite PK profile.

In some embodiments, the method further comprises producing the oral drug dosage form. In some embodiments, the oral drug dosage form is produced by three-dimensional printing. In some embodiments, the three-dimensional printing is carried out by fused deposition modeling (FDM). In some embodiments, the three-dimensional printing is carried out by melt extrusion deposition (MED).

In another aspect, the present disclosure provides a method of three-dimensional printing of an oral drug dosage form designed according to any one of the methods described herein.

In another aspect, the present disclosure provides an oral drug dosage form produced by the methods described herein.

In another aspect, the present disclosure provides an oral drug dosage form comprising a fixed amount of a drug formulated and configured to have a desired composite pharmacokinetic (PK) profile, the oral drug dosage form having two dosage units stacked back-to-back, wherein each dosage unit comprises: a first modulated-release (MR1) portion comprising the drug; a second modulated-release (MR2) portion comprising the drug; and a shell, wherein the MR1 portion has a top surface and a bottom surface, wherein the MR2 portion has a top surface and a bottom surface, wherein the shell partially surrounds the MR1 portion and the MR2 portion, and wherein the shell is in direct contact with both the MR1 portion and the MR2 portion and leaves one surface of the MR1 portion and/or one surface of the MR2 portion exposed. In some embodiments, the MR1 portion is an IR portion, the IR portion having an immediate-release profile, and the MR2 portion is an extended release (ER) portion comprising the drug, the ER portion having an extended-release profile. In some embodiments, the MR1 portion is a first ER portion, the first ER portion having an extended-release profile, and the MR2 portion is a second extended release (ER) portion comprising the drug, the second ER portion having an extended-release profile. In some embodiments, the drug has linear pharmacokinetics. In some embodiments, the shell is non-erodible. In some embodiments, the MR1 portion and the MR2 portion are stacked on top of each other. In some embodiments, the MR1 portion and the MR2 portion are positioned side-by-side with each other. In some embodiments, at least in one of the dosage units, the MR1 portion is stacked on top of the MR2 portion, and wherein the shell leaves only the top surface of the MR1 portion exposed. In some embodiments, the bottom surface of the MR1 portion is in direct contact with the top surface of the MR2 portion. In some embodiments, the dosage unit further comprises a third modulated-release (MR3) portion. In some embodiments, the MR3 portion is an IR portion. In some embodiments, the MR3 portion is an ER portion. In some embodiments, the MR3 portion is an IR portion, wherein the MR3 portion has a top surface and a bottom surface, wherein the MR2 portion is stacked on top of the MR3 portion, and wherein the shell leaves only the top surface of the MR1 portion exposed. In some embodiments, the bottom surface of the MR2 portion is in direct contact with the top surface of the MR3 portion. In some embodiments, at least in one of the dosage units, the MR2 portion is stacked on top of the MR1 portion, and wherein the shell leaves only the top surface of the MR2 portion exposed. In some embodiments, the bottom surface of the MR2 portion is in direct contact with the top surface of the MR1 portion. In some embodiments, at least in one of the dosage units, the MR1 portion and the MR2 portion are positioned side-by-side with each other, and wherein the shell leaves the top surface of both the MR1 portion and the MR2 portion exposed. In some embodiments, the two dosage units are the same. In some embodiments, the two dosage units are different. In some embodiments, substantially all of the MR1 portion erodes within at least about 20 minutes following administration of the oral drug dosage form to an individual. In some embodiments, the MR1 portion comprises an erodible material. In some embodiments, the MR2 portion comprises an erodible material, and wherein the drug contained in the MR2 portion is released from the oral drug dosage form over a period of at least about 6 hours.

In another aspect, the present disclosure provides an oral drug dosage form comprising a fixed amount of a drug formulated and configured to have a desired composite pharmacokinetic (PK) profile, the oral drug dosage form having two dosage units stacked back-to-back, wherein each dosage unit comprises: an immediate-release (IR) portion comprising the drug, the IR portion having an immediate-release profile: an extended release (ER) portion comprising the drug, the ER portion having an extended-release profile; and a shell, wherein the IR portion has a top surface and a bottom surface, wherein the ER portion has a top surface and a bottom surface, wherein the shell partially surrounds the IR portion and the ER portion, and wherein the shell is in direct contact with both the IR portion and the ER portion and leaves one surface of the IR portion and/or one surface of the ER portion exposed. In some embodiments, the drug has linear pharmacokinetics. In some embodiments, the shell is non-erodible. In some embodiments, the IR portion and the ER portion are stacked on top of each other. In some embodiments, the IR portion and the ER portion are positioned side-by-side with each other. In some embodiments, at least in one of the dosage units, the JR portion is stacked on top of the ER portion, and wherein the shell leaves only the top surface of the IR portion exposed. In some embodiments, the bottom surface of the IR portion is in direct contact with the top surface of the ER portion. In some embodiments, the dosage unit further comprises a second JR portion, wherein the second JR portion has a top surface and a bottom surface, wherein the ER portion is stacked on top of the second IR portion, and wherein the shell leaves only the top surface of the IR portion exposed. In some embodiments, the bottom surface of the ER portion is in direct contact with the top surface of the second IR portion. In some embodiments, at least in one of the dosage units, the ER portion is stacked on top of the IR portion, and wherein the shell leaves only the top surface of the ER portion exposed. In some embodiments, the bottom surface of the ER portion is in direct contact with the top surface of the IR portion. In some embodiments, at least in one of the dosage units, the IR portion and the ER portion are positioned side-by-side with each other, and wherein the shell leaves the top surface of both the JR portion and the ER portion exposed. In some embodiments, the two dosage units are the same. In some embodiments, the two dosage units are different. In some embodiments, substantially all of the JR portion erodes within at least about 20 minutes following administration of the oral drug dosage form to an individual. In some embodiments, the IR portion comprises an erodible material. In some embodiments, the ER portion comprises an erodible material, and wherein the drug contained in the ER portion is released from the oral drug dosage form over a period of at least about 6 hours.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### BRIEF DESCRIPTION OF THE DRAWINGS

FIGS. 1A-1J show exemplary dosage units and oral drug dosage forms.

FIGS. 2A-2E show deconstructed and deconstructed, cross-sectional views of exemplary dosage units and oral drug dosage forms.

FIG. 3 is a schematic of an exemplary workflow of the 3D Printing Formulation by Design (3DPFbD®) approach.

FIGS. 4A-4E show schematics of an exemplary oral drug dosage form comprising an extended-release (ER) portion, an immediate-release (IR) portion, and a shell. FIG. 4A depicts a deconstructed view of the oral drug dosage form to show the individual components thereof. FIG. 4B depicts the assembled oral drug dosage form. FIG. 4C is a schematic showing dimensional aspects of the oral drug dosage form. FIG. 4D shows the compositions of the components of the oral drug dosage form. FIG. 4E is a cross-sectional view of the oral drug dosage form.

FIG. 5 shows in vivo pharmacokinetic curves of an IR precursor drug dosage form and an IR reference drug dosage form.

FIG. 6 shows an in vivo pharmacokinetic curve of an ER precursor drug dosage form.

FIG. 7 shows theoretical simulated PK curves of oral drug dosage forms having different IR:ER drug ratios as compared to the PK curve of a reference drug.

FIG. 8 shows in vivo pharmacokinetic curves of a whole oral drug dosage form having 100 mg of the drug, an IR reference drug dosage form having 50 mg of the drug, and an ER reference drug dosage form having 100 mg of the drug.

FIG. 9 shows in intro dissolution rates of a whole oral drug dosage form having 100 mg of the drug and an ER reference drug dosage form having 100 mg of the drug.

FIG. 10 shows an in vivo pharmacokinetic curve of an optimized oral drug dosage form.

FIG. 11 shows in vivo pharmacokinetic curves of an ER precursor drug dosage form having 100 mg of the drug and an ER reference drug dosage form having 100 mg of the drug.

FIG. 12 shows in vivo pharmacokinetic curves of an optimized 3D-printed oral drug dosage form (100 mg of the drug), the theoretical prediction of the 3D-printed oral drug dosage form (100 mg of the drug), an ER reference drug dosage form (100 mg of the drug), and an IR reference drug dosage form (50 mg of the drug).

FIG. 13A shows a deconstructed view of an oral drug dosage form 1400 comprising an ER portion comprising a drug 1405, an IR portion comprising the drug 1410, and a shell 1415. FIG. 13B shows PK curves of an IR precursor drug dosage form and an ER precursor drug dosage form of the oral drug dosage form. FIG. 13C shows theoretical simulated PK curves of oral drug dosage forms having different IR:ER drug ratios. FIG. 13D shows an in vivo PK curve of an oral drug dosage form having an IR:ER drug ratio of 1:1 as compared to the theoretical simulated PK curve of an oral drug dosage form having an IR ER drug ratio of 1:1.

### DETAILED DESCRIPTION

The present disclosure provides novel methods for designing an oral drug dosage form having a fixed amount of a drug, the oral drug dosage form comprising a dosage unit comprising: a first modulated-release (MR1) portion (such as an immediate-release (IR) portion) comprising the drug; and a second modulated-release (MR2) portion (such as an extended-release (ER) portion) comprising the drug, designed to meet a desired composite PK profile in an individual based on a MR1 PK curve of a MR1 precursor drug dosage form comprising the MR1 portion and a MR2 PK curve of a MR2 precursor drug dosage form comprising the MR2 portion. As demonstrated herein, the inventors have discovered that such oral drug dosage forms can be designed by determining the relative amounts of the drug in the MR1 portion, such as an IR portion, and the MR2 portion, such as an ER portion, of the dosage unit based on PK data, such as a MR1 PK curve and/or a MR2 PK curve of precursor drug dosage forms, such that when the MR1 portion and the MR2 portion are combined together to form the oral drug dosage form, the desired composite PK profile is obtained. In some aspects, the methods described herein may be applied to designing an oral drug dosage form having a fixed amount of a drug and a desired composite PK profile in an individual, wherein the oral drug dosage form comprises a dosage unit comprising more than one ER portion and/or more than one IR portion. In some aspects, using the methods described herein, oral drug dosage forms may be designed to be bioequivalent to a reference drug dosage form or a reference administration regimen. The oral drug dosage forms designed using the methods described herein may be readily printed using three-dimensional printing (3D) techniques or manufacturing techniques comprising 3D printing techniques. Such drug dosage forms may be designed to, e.g., improve treatment efficacy, reduce toxicity, and increase patient compliance by, e.g., designing an oral drug dosage form for a once-daily dosing regimen that is bioequivalent with a regimen that involves administration of a drug dosage form two or more times per day. Further provided herein are novel oral drug dosage forms, such as oral drug dosage forms produced by the methods described herein.

Presented herein is a novel 3D Printing Formulation by Design (3DPFbD®) approach for designing oral drug dosage forms (including those having a complex geometric structure) having a desired pharmacokinetic profile. The 3DPFbD® approach, which utilizes multi-portioned designs, provides a method for producing customizable and easily optimizable 3D-printed solid drug dosage forms having a desired PK profile, and thus can be used to efficiently and effectively design and fabricate a drug delivery system. As demonstrated herein, the 3DPFbD® method described herein can be used to design modified release dosage forms with predetermined in vivo release profiles. This innovative approach provides, e.g., means for preclinical and clinical trial formulation development on a predictable and accelerated timeline. The methods described herein are examples of the 3DPFbD® approach.

Although much of the application discusses oral drug dosage forms, one of ordinary skill in the art will readily understand that this disclosure also applies and pertains to other oral dosage forms configured and formulated to provide a desired PK profile of any compound, such as a dosage form comprising a reagent (e.g., an oral reagent dosage form).

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, unless otherwise noted, “rate of release” or “release rate” of a drug refers to the quantity of drug released from a dosage form per unit time, e.g., milligrams of drug released per hour (mg/hour) or a percentage of a total drug dose released per hour. Drug release rates for dosage forms are typically measured as an in vitro rate of drug release, e.g., a quantity of drug released from the dosage form per unit time measured under appropriate conditions and in a suitable fluid.

A “zero-order release profile” characterizes the release profile of a dosage form that releases a constant amount of drug per unit time. A pseudo-zero order release profile is one that approximates a zero-order release profile. A dissolution curve shows a zero or pseudo-zero order release profile if its release rate remains constant (or relatively constant within ±10% of the average value) in the interval of time 0≤a<t≤b. Any profile following the equation: (M(t)/Mₜ)=k(t−a)ⁿ0≤n≤1.1 has the following release rate equation: (1/M)(dM/dt)=kn(t−a)ⁿ⁻¹.

A “first order release profile” characterizes the release profile of a dosage form that releases a percentage of a drug charge per unit time. A pseudo-first order release profile is one that approximates a first order release profile. A dissolution curve shows a first or pseudo-first order release profile within a certain interval of time 0≤a<t≤b if its release rate is a continued monotone decreasing function of time. Specifically, a dissolution curve shows a first order profile whenever its release rate is proportional to the remaining undissolved amount of drug, as determined by the following equation: (M(t)/MT)=1−exp(−kt). A dissolution curve shows a pseudo-first order profile when the drug release rate decreases with time as described by the Fickian or anomalous Fickian diffusion controlled release equation: (MW/M_{T})=ktⁿ, 0.3≤n≤0.7.

The maximum plasma drug concentration during the dosing period is referenced as Cₘₐₓ, while Cₘᵢₙ refers to the minimum blood plasma drug concentration at the end of a dosing interval; and Cₐᵥₑ refers to an average concentration during the dosing interval. The “degree of fluctuation” is defined as a quotient (Cₘₐₓ−Cᵢₙ)/Cₐᵥₑ.

Persons of skill in the art will appreciate that blood plasma drug concentrations obtained in individual subjects will vary due to interpatient variability in the many parameters affecting drug absorption, distribution, metabolism and excretion. For this reason, unless otherwise indicated, when a drug plasma concentration is listed, the value listed is the calculated mean value based on values obtained from a groups of subjects tested.

The term “bioavailability” refers to an extent to which—and sometimes rate at which—the active moiety (drug or metabolite) enters systemic circulation, thereby gaining access to the site of action.

“AUC” is the area under the plasma concentration-time curve and is considered to be the most reliable measure of bioavailability. It is directly proportional to the total amount of unchanged drug that reaches the systemic circulation.

As used herein, “treat,” “treatment,” or “treating” is an approach for obtaining beneficial or desired results including clinical results. For purposes of this disclosure, beneficial or desired clinical results include, but are not limited to, one or more of the following: alleviating one or more symptoms resulting from the disease, decreasing the dose of one or more other medications required to treat the disease, and/or increasing the quality of life.

As used herein, the term “individual” refers to a mammal and includes, but is not limited to, human, bovine, horse, feline, canine, rodent, rat, mouse, dog, or primate. In some embodiments, the individual is human.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### Methods of Designing an Oral Drug Dosage Form

The present disclosure provides, in some aspects, methods of designing an oral drug dosage form described herein having a fixed amount of a drug and a desired composite pharmacokinetic (PK) profile in an individual, wherein the oral drug dosage form comprises at least one dosage unit comprising: a MR1 portion (such as an IR portion or an ER portion) comprising the drug; and a MR2 portion (such as an IR portion or an ER portion) comprising the drug. The modulated-release portions described herein may have any drug release characteristic, such as an immediate-release profile or an extended-release profile, suitable for designing an oral drug dosage form having a desired composite PK profile in an individual. In some embodiments, the oral drug dosage form comprises a dosage unit comprising: an immediate-release (IR) portion comprising the drug, the IR portion having an immediate-release profile; and an extended-release (ER) portion comprising the drug, the ER portion having an extended-release profile. In some embodiments, the oral drug dosage form comprises a dosage unit comprising: a first ER portion comprising the drug, the first ER portion having an extended-release profile; and a second ER portion comprising the drug, the second ER portion having an extended-release profile. In some embodiments, the dosage unit further comprises another component, such as another modulated-release portion, e.g., an IR portion (such as an IR layer) or an ER portion (such as an ER layer), an intermediate portion, or a shell.

For purposes of brevity, in many embodiments disclosed herein a dosage form comprising an IR portion (as the MR1 portion) and an ER portion (as the MR2 portion) is described to exemplify the invention. This disclosure is not to be understood as limiting the description herein and such teachings can also be applied to other configurations wherein the MR1 portion and/or the MR2 portion are different modulated-release portions.

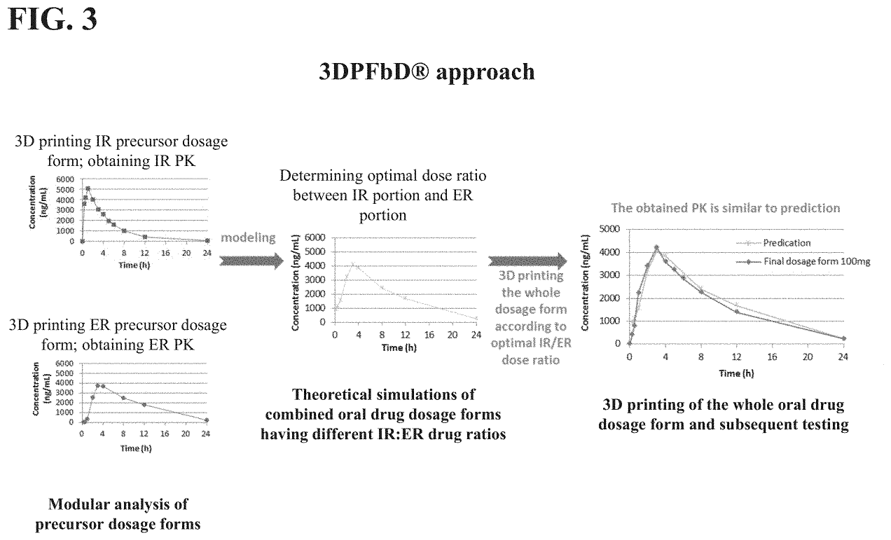

An exemplary schematic of the 3DPFbD® approach described herein is provided in FIG. 3. Specifically, in some embodiments, the method comprises: a modular PK analysis of precursor dosage forms, such as an IR precursor drug dosage form and an ER precursor drug dosage form; theoretical simulations based on the modular PK analysis for one or more combined oral drug dosage forms having a drug ratio of the IR portion and the ER portion (IR:ER drug ratio); and subsequent steps, such as 3D printing an oral drug dosage form and in vivo and/or in vitro testing.

In some embodiments, the method comprises: determining the relative amounts of the drug in the MR1 portion and the MR2 portion based on a MR1 PK curve of a MR1 precursor drug dosage form comprising the MR1 portion in the individual, and a MR2 PK curve of a MR2 precursor drug dosage form comprising the MR2 portion in the individual such that the MR1 portion and the MR2 portion when combined together produce the oral drug dosage form having the desired composite PK profile in the individual. In some embodiments, the method comprises: (a) obtaining a MR1 PK curve of a MR1 precursor drug dosage form comprising the MR1 portion in the individual; (b) obtaining a MR2 PK curve of a MR2 precursor drug dosage form comprising the MR2 portion in the individual; and (c) determining the relative amounts of the drug in the MR1 portion and the MR2 portion based on the MR1 PK curve and the MR2 PK curve such that the MR1 portion and the MR2 portion when combined together produce the oral drug dosage form having the desired composite PK profile in the individual. In some embodiments, the drug has linear pharmacokinetics.

In some embodiments, the method comprises: determining the relative amounts of the drug in the IR portion and the ER portion based on an IR PK curve of an IR precursor drug dosage form comprising the IR portion in the individual, and an ER PK curve of an ER precursor drug dosage form comprising the ER portion in the individual such that the IR portion and the ER portion when combined together produce the oral drug dosage form having the desired composite PK profile in the individual. In some embodiments, the method comprises: (a) obtaining an IR PK curve of an JR precursor drug dosage form comprising the IR portion in the individual; (b) obtaining an ER PK curve of an ER precursor drug dosage form comprising the ER portion in the individual; and (c) determining the relative amounts of the drug in the IR portion and the ER portion based on the IR PK curve and ER PK curve such that the IR portion and the ER portion when combined together produce the oral drug dosage form having the desired composite PK profile in the individual. In some embodiments, the drug has linear pharmacokinetics.

In other aspects, the present disclosure provides a method of designing an oral drug dosage form having a fixed amount of a drug and a desired composite pharmacokinetic (PK) profile in an individual, wherein the oral drug dosage form comprises a dosage unit comprising: an immediate-release (IR) portion comprising the drug, the IR portion having an immediate-release profile; and an extended-release (ER) portion comprising the drug, the ER portion having an extended-release profile, wherein the IR portion and the ER portion are stacked on top of each other, and wherein, at the fixed amount of the drug, the drug has linear pharmacokinetics, the method comprising: (a) obtaining an IR PK curve of an IR precursor drug dosage form comprising the IR portion in the individual; (b) obtaining an ER PK curve of an ER precursor drug dosage form comprising the ER portion in the individual, and (c) determining the relative amounts of the drug in the IR portion and the ER portion based on the IR PK curve and ER PK curve such that the IR portion and the ER portion when combined together produce the oral drug dosage form having the desired composite PK profile in the individual.

In other aspects, the present disclosure provides a method of designing an oral drug dosage form having a fixed amount of a drug and a desired composite pharmacokinetic (PK) profile in an individual, wherein the oral drug dosage form comprises a dosage unit comprising: a first extended-release (ER) portion comprising the drug, the first ER portion having an extended-release profile; and a second extended-release (ER) portion comprising the drug, the second ER portion having an extended-release profile, wherein the first ER portion and the second ER portion are stacked on top of each other, and wherein, at the fixed amount of the drug, the drug has linear pharmacokinetics, the method comprising: (a) obtaining a first ER PK curve of a first ER precursor drug dosage form comprising the first ER portion in the individual; (b) obtaining a second ER PK curve of a second ER precursor drug dosage form comprising the second ER portion in the individual; and (c) determining the relative amounts of the drug in the first ER portion and the second ER portion based on the first ER PK curve and the second ER PK curve such that the first ER portion and the second ER portion when combined together produce the oral drug dosage form having the desired composite PK profile in the individual.

In other aspects, the present disclosure provides a method of designing an oral drug dosage form having a fixed amount of a drug and a desired composite pharmacokinetic (PK) profile in a human, wherein the oral drug dosage form comprises a dosage unit comprising: an immediate-release (IR) portion comprising the drug, the IR portion having an immediate-release profile; and an extended-release (ER) portion comprising the drug, the ER portion having an extended-release profile, wherein the IR portion and the ER portion are stacked on top of each other, and wherein, at the fixed amount of the drug, the drug has linear pharmacokinetics, the method comprising: (a) obtaining an IR PK curve of an IR precursor drug dosage form comprising the IR portion in the human; (b) obtaining an ER PK curve of an ER precursor drug dosage form comprising the ER portion in the human; and (c) determining the relative amounts of the drug in the IR portion and the ER portion based on the IR PK curve and ER PK curve such that the IR portion and the ER portion when combined together produce the oral drug dosage form having the desired composite PK profile in the human.

In other aspects, the present disclosure provides a method of designing an oral drug dosage form having a fixed amount of a drug and a desired composite pharmacokinetic (PK) profile in an individual, wherein the oral drug dosage form comprises a dosage unit comprising: an immediate-release (R) portion comprising the drug, the IR portion having an immediate-release profile; and an extended-release (ER) portion comprising the drug, the ER portion having an extended-release profile, wherein the IR portion and the ER portion are stacked on top of each other, and wherein, at the fixed amount of the drug, the drug has linear pharmacokinetics, the method comprising: (a) obtaining an IR PK curve of an IR precursor drug dosage form comprising the JR portion in the individual; (b) obtaining an ER PK curve of an ER precursor drug dosage form comprising the ER portion in the individual; and (c) determining the relative amounts of the drug in the JR portion and the ER portion based on the JR PK curve and ER PK curve such that the IR portion and the ER portion when combined together produce the oral drug dosage form having the desired composite PK profile in the individual, wherein the oral drug dosage form has the desired composite PK profile in the individual for between 0 hours and about 24 hours.

In other aspects, the present disclosure provides a method of designing an oral drug dosage form having a fixed amount of a drug and a desired composite pharmacokinetic (PK) profile in an individual, wherein the oral drug dosage form comprises a dosage unit comprising: an immediate-release (IR) portion comprising the drug, the IR portion having an immediate-release profile; and an extended-release (ER) portion comprising the drug, the ER portion having an extended-release profile, wherein the IR portion and the ER portion are stacked on top of each other, and wherein the desired composite PK profile is determined based on having an area under the curve (AUC), a Cₘₐₓ, and a tₘₐₓ within an acceptable threshold of a reference PK curve of the drug, the method comprising: (a) obtaining an IR PK curve of an IR precursor drug dosage form comprising the IR portion in the individual; (b) obtaining an ER PK curve of an ER precursor drug dosage form comprising the ER portion in the individual; and (c) determining the relative amounts of the drug in the IR portion and the ER portion based on the IR PK curve and ER PK curve such that the IR portion and the ER portion when combined together produce the oral drug dosage form having the desired composite PK profile in the individual. In some embodiments, the drug has linear pharmacokinetics.

In other aspects, the present disclosure provides a method of designing an oral drug dosage form having a fixed amount of a drug and a desired composite pharmacokinetic (PK) profile in an individual, wherein the oral drug dosage form comprises a dosage unit comprising: an immediate-release (IR) portion comprising the drug, the IR portion having an immediate-release profile; and an extended-release (ER) portion comprising the drug, the ER portion having an extended-release profile, wherein the IR portion and the ER portion are positioned side-by-side, wherein, at the fixed amount of the drug, the drug has linear pharmacokinetics, the method comprising: (a) obtaining an IR PK curve of an IR precursor drug dosage form comprising the JR portion in the individual; (b) obtaining an ER PK curve of an ER precursor drug dosage form comprising the ER portion in the individual; and (c) determining the relative amounts of the drug in the IR portion and the ER portion based on the IR PK curve and ER PK curve such that the IR portion and the ER portion when combined together produce the oral drug dosage form having the desired composite PK profile in the individual.

In other aspects, the present disclosure provides a method of designing an oral drug dosage form having a fixed amount of a drug and a desired composite pharmacokinetic (PK) profile in an individual, wherein the oral drug dosage form comprises a dosage unit comprising: a first extended-release (ER) portion comprising the drug, the first ER portion having an extended-release profile; and a second extended-release (ER) portion comprising the drug, the second ER portion having an extended-release profile, wherein the first ER portion and the second ER portion are positioned side-by-side, wherein, at the fixed amount of the drug, the drug has linear pharmacokinetics, the method comprising: (a) obtaining a first ER PK curve of a first ER precursor drug dosage form comprising the first ER portion in the individual; (b) obtaining a second ER PK curve of a second ER precursor drug dosage form comprising the second ER portion in the individual, and (c) determining the relative amounts of the drug in the first ER portion and the second ER portion based on the first ER PK curve and second ER PK curve such that the first ER portion and the second ER portion when combined together produce the oral drug dosage form having the desired composite PK profile in the individual.

In other aspects, the present disclosure provides a method of designing an oral drug dosage form having a fixed amount of a drug and a desired composite pharmacokinetic (PK) profile in a human, wherein the oral drug dosage form comprises a dosage unit comprising: an immediate-release (IR) portion comprising the drug, the IR portion having an immediate-release profile; and an extended-release (ER) portion comprising the drug, the ER portion having an extended-release profile, wherein the IR portion and the ER portion are positioned side-by-side, wherein, at the fixed amount of the drug, the drug has linear pharmacokinetics, the method comprising: (a) obtaining an IR PK curve of an IR precursor drug dosage form comprising the JR portion in the human; (b) obtaining an ER PK curve of an ER precursor drug dosage form comprising the ER portion in the human; and (c) determining the relative amounts of the drug in the IR portion and the ER portion based on the JR PK curve and ER PK curve such that the IR portion and the ER portion when combined together produce the oral drug dosage form having the desired composite PK profile in the human.

In other aspects, the present application provides a method of determining the relative amounts of a drug in an immediate-release (IR) portion and an extended-release (ER) portion of an oral drug dosage form having a fixed amount of a drug and a desired composite pharmacokinetic (PK) profile in an individual, wherein the oral drug dosage form comprises a dosage unit comprising: the IR portion comprising the drug, the IR portion having an immediate-release profile; and the ER portion comprising the drug, the ER portion having an extended-release profile, the method comprising, determining the relative amounts of the drug in the IR portion and the ER portion based on an IR PK curve of an IR precursor drug dosage form comprising the IR portion in the individual, and an ER PK curve of an ER precursor drug dosage form comprising the ER portion in the individual such that the IR portion and the ER portion when combined together produce the oral drug dosage form having the desired composite PK profile in the individual. In some embodiments, the drug has linear pharmacokinetics.

In other aspects, the present application provides a method of determining the relative amounts of a drug in an immediate-release (IR) portion and an extended-release (ER) portion of an oral drug dosage form having a fixed amount of a drug and a desired composite pharmacokinetic (PK) profile in an individual, wherein the oral drug dosage form comprises a dosage unit comprising: the JR portion comprising the drug, the IR portion having an immediate-release profile; and the ER portion comprising the drug, the ER portion having an extended-release profile, wherein the IR portion and the ER portion are stacked on top of each other, the method comprising, determining the relative amounts of the drug in the IR portion and the ER portion based on an IR PK curve of an JR precursor drug dosage form comprising the IR portion in the individual, and an ER PK curve of an ER precursor drug dosage form comprising the ER portion in the individual such that the IR portion and the ER portion when combined together produce the oral drug dosage form having the desired composite PK profile in the individual. In some embodiments, the drug has linear pharmacokinetics.

In other aspects, the present application provides a method of determining the relative amounts of a drug in an immediate-release (IR) portion and an extended-release (ER) portion of an oral drug dosage form having a fixed amount of a drug and a desired composite pharmacokinetic (PK) profile in an individual, wherein the oral drug dosage form comprises a dosage unit comprising: the IR portion comprising the drug, the IR portion having an immediate-release profile; and the ER portion comprising the drug, the ER portion having an extended-release profile, wherein the JR portion and the ER portion are positioned side-by-side, the method comprising, determining the relative amounts of the drug in the IR portion and the ER portion based on an IR PK curve of an IR precursor drug dosage form comprising the IR portion in the individual, and an ER PK curve of an ER precursor drug dosage form comprising the ER portion in the individual such that the IR portion and the ER portion when combined together produce the oral drug dosage form having the desired composite PK profile in the individual. In some embodiments, the drug has linear pharmacokinetics.

In other aspects, the present application provides a method of producing an oral drug dosage form having a fixed amount of a drug and a desired composite pharmacokinetic (PK) profile in an individual, wherein the oral drug dosage form comprises a dosage unit comprising: the IR portion comprising the drug, the IR portion having an immediate-release profile; and the ER portion comprising the drug, the ER portion having an extended-release profile, the method comprising: determining the relative amounts of the drug in the IR portion and the ER portion based on an IR PK curve of an IR precursor drug dosage form comprising the IR portion in the individual, and an ER PK curve of an ER precursor drug dosage form comprising the ER portion in the individual such that the IR portion and the ER portion when combined together produce the oral drug dosage form having the desired composite PK profile in the individual. In some embodiments, the drug has linear pharmacokinetics.

In some aspects, the methods disclosed herein further comprise producing the oral drug dosage form, such as by three-dimensional printing or a manufacturing technique comprising a 3D printing technique, such as 3D printing in combination with another method, e.g., a combination of injection molding and 3D printing.

In some embodiments, the MR1 PK curve of a MR1 precursor drug dosage form comprising the MR1 portion in the individual and/or the MR2 PK curve of a MR2 precursor drug dosage form comprising the MR2 portion in the individual are determined prior to determining the relative amounts of the drug in the MR1 portion and the MR2 portion.

In some embodiments, the MR1 PK curve and the MR2 PK curve are obtained from the same species of individual. In some embodiments, the MR1 PK curve and the MR2 PK curve are obtained from the same individual, wherein administration of the MR1 precursor drug dosage form and MR2 precursor drug dosage form are separated by an appropriate period of time to allow clearance of the drug, e.g., at least about 5 drug half-lives.

In some embodiments, determining the relative amounts of the drug in the MR1 portion and the MR2 portion is based on a point-to-point comparison between the MR1 PK curve and/or MR2 PK curve and a desired composite PK curve. In some embodiments, the determining of the relative amounts of the drug in the MR1 portion and the MR2 portion is based on drug in vivo dynamic information.

### Oral Drug Dosage Forms and Dosage Units

In some aspects, provided herein are oral drug dosage forms and dosage units having a fixed amount of a drug and a desired composite pharmacokinetic (PK) profile in an individual. In some embodiments, the oral drug dosage form is designed according to the methods described herein. In some embodiments, the dosage unit is designed according to the methods described herein.

In some embodiments, the oral drug dosage form comprises a dosage unit comprising a MR1 portion (such as an IR portion having an immediate-release profile or an ER portion having an extended-release profile) comprising the drug, and a MR2 portion (such as an ER portion having an extended-release profile) comprising the drug. In some embodiments, the oral drug dosage form comprises a dosage unit comprising an IR portion, such as an IR layer, comprising the drug, the IR portion having an immediate-release profile, and an ER portion, such as an ER layer, comprising the drug, the ER portion having an extended-release profile. In some embodiments, the oral drug dosage form comprises a dosage unit comprising a first ER portion, such as an ER layer, comprising the drug, the first ER portion having an extended-release profile, and a second ER portion, such as an ER layer, comprising the drug, the second ER portion having an extended-release profile. In some embodiments, the term “dosage unit” refers to a portion of an oral drug dosage form comprising an IR portion and an ER portion. In some embodiments of the description herein, the term dosage unit may be used to describe and/or test a portion of an oral drug dosage form to, e.g., simplify design and/or testing of the oral drug dosage form. For example, in some embodiments wherein an oral drug dosage form comprises two or more of the same dosage unit, the design of the oral drug dosage form may be based on testing of a single dosage unit. In some embodiments, the dosage unit further comprises other components, such as another IR portion, another ER portion, a shell, or an intermediate portion. In some embodiments, when the oral drug dosage form comprises only one dosage unit, the terms oral drug dosage form and dosage unit may be used interchangeably to describe the dosage form.

The orientation of dosage units of an oral drug dosage form described herein may be assembled in a multitude of orientations relative to one another. In some embodiments, to facilitate the description of oral drug dosage forms within the scope of the present disclosure, the orientation of a first dosage unit relative to another dosage unit may be described based on surfaces of the dosage units that do not release the drug, e.g., a shell surface, or are not exposed to, e.g., GI fluid following administration. For example, in some embodiments, two dosage units that are positioned, such as stacked, back-to-back interface with one another at surfaces of each dosage unit that do not release the drug. In some embodiments, the orientation of two dosage units of an oral drug dosage form may be described as positioned side-by-side, wherein the two dosage units interface with one another at surfaces of each dosage unit that do not release the drug or are not exposed to, e.g., GI fluid following administration, wherein each dosage unit comprises a top surface from which the drug is release or is exposed to, e.g., GI fluid, and wherein the top surfaces of the dosage units are on the same surface of the oral drug dosage form. In some embodiments, due to, e.g., the nature of 3D printing, the delimitation between two dosage units of an oral drug dosage form is arbitrary.

In some embodiments, the oral drug dosage form comprises a single dosage unit. In some embodiments, the oral drug dosage form comprises more than one, such as any of 2, 3, 4, 5, or 6, dosage units described herein. In some embodiments, wherein the oral drug dosage form comprises more than one dosage unit, each dosage unit is the same. In some embodiments, wherein the oral drug dosage form comprises more than one dosage unit, at least one dosage unit is different from the other dosage units of the oral drug dosage form.

In some embodiments, the oral drug dosage form comprises two dosage units. In some embodiments, the two dosage units are the same. In some embodiments, wherein the oral drug dosage form comprises two dosage units, the two dosage units are different. In some embodiments, the two dosage units are stacked back-to-back. In some embodiments, wherein the two dosage units are stacked back-to-back, the drug is released from a first dosage unit on a first side of the oral drug dosage form and the drug is released from a second dosage unit on a second side of the oral drug dosage form. In some embodiments, the two dosage units are positioned side-by-side. One of ordinary skill in the art will readily appreciate that the oral drug dosage forms described herein may have a wide variety of configurations, including more complex arrangements of dosage units and oral drug dosage forms comprising a plurality of dosage units. For example, in some embodiments, the oral drug dosage form comprises four dosage units, wherein the first dosage unit and the second dosage unit are positioned side-by-side, wherein the third dosage unit and the fourth dosage unit are positioned side-by-side, and wherein the first and second dosage units are stacked back-to-back with the third and fourth dosage units.

In some embodiments, dosage units of an oral drug dosage form are separated, in whole or in part, by a component, e.g., a shell or an intermediate portion. In some embodiments, wherein the oral drug dosage form comprises two dosage units stacked back-to-back, the two dosage units are separated, in whole or in part, by a component, e.g., a shell or an intermediate portion. In some embodiments, wherein the oral drug dosage form comprises two dosage units position side-by-side, the two dosage units are separated, in whole or in part, by a component, e.g., a shell or an intermediate portion.

In some embodiments, the oral drug dosage form comprises one or more drugs. In some embodiments, wherein the oral drug dosage form comprises more than one dosage unit, dosage units of the oral drug dosage form may comprise different drugs or drug combinations. In some embodiments, wherein the oral drug dosage form comprises a first dosage unit and a second dosage unit, the first dosage unit comprises a different drug or drug combination than the second dosage unit. In some embodiments, the oral drug dosage form comprises one drug.

In some embodiments, the oral drug dosage unit is suitable for oral administration. The drug dosage forms of the present invention can be, for example, any size, shape, or weight that is suitable for oral administration to specific individuals, such as children and adults. In some embodiments, the selection of size, shape, or weight of the oral drug dosage form is based on an attribute of an individual to receive administration of the oral drug dosage form. In some embodiments, the attribute of the individual is one or more of height, weight, or age. In some embodiments, the shape of the oral drug dosage form comprises a cylinder, oval, bullet shape, arrow head shape, triangle, arced triangle, square, arced square, rectangle, arced rectangle, diamond, pentagon, hexagon, octagon, half moon, almond, or a combination thereof. In some embodiments, the size and shape of the oral drug dosage form is suitable for oral administration to the individual.

In some embodiments, the oral drug dosage form has a dimension that is less than about 22 mm, such as less than about 21 mm, less than about 20 mm, less than about 19 mm, less than about 18 mm, less than about 17 mm, less than about 16 mm, less than about 15 mm, less than about 14 mm, less than about 13 mm, less than about 12 mm, less than about 11 mm, less than about 10 mm, less than about 9 mm, less than about 8 mm, less than about 7 mm, less than about 6 mm, less than about 5 mm, less than about 4 mm, less than about 3 mm, less than about 2 mm, or less than about 1 mm. In some embodiments, the drug dosage form has a dimension that is about 1 mm to about 22 mm, such as about 21 mm, about 20 mm, about 19 mm, about 18 mm, about 17 mm, about 16 mm, about 15 mm, about 14 mm, about 13 mm, about 12 mm, about 11 mm, about 10 mm, about 9 mm, about 8 mm, about 7 mm, about 6 mm, about 5 mm, about 4 mm, about 3 mm, or about 2 mm.

In some embodiments, the fixed amount of the drug in an oral drug dosage form is between about 2000 mg to about 0.01 mg. In some embodiments, the fixed amount of the drug in an oral dosage form is less than about 2000 mg, such as less than about any of 1900 mg, 1800 mg, 1700 mg, 1600 mg, 1500 mg, 1400 mg, 1300 mg, 1200 mg, 1100 mg, 1000 mg, 900 mg, 800 mg, 700 mg, 600 mg, 500 mg, 450 mg, 400 mg, 350 mg, 300 mg, 250 mg, 200 mg, 150 mg, 100 mg, 75 mg, 50 mg, 45 mg, 40 mg, 35 mg, 30 mg, 25 mg, 20 mg, 15 mg, 10 mg, 5 mg, 4 mg, 3 mg, 2 mg, 1 mg, 0.75 mg, 0.5 mg, 0.25 mg, or 0.1 mg. In some embodiments, the fixed amount of the drug in an oral dosage form is about 2000 mg, such as about any of 1900 mg, 1800 mg, 1700 mg, 1600 mg, 1500 mg, 1400 mg, 1300 mg, 1200 mg, 1100 mg, 1000 mg, 900 mg, 800 mg, 700 mg, 600 mg, 500 mg, 450 mg, 400 mg, 350 mg, 300 mg, 250 mg, 200 mg, 150 mg, 100 mg, 75 mg, 50 mg, 45 mg, 40 mg, 35 mg, 30 mg, 25 mg, 20 mg, 15 mg, 10 mg, 5 mg, 4 mg, 3 mg, 2 mg, 1 mg, 0.75 mg, 0.5 mg, 0.25 mg, or 0.1 mg.

In some embodiments, the drug dosage form has a total weight of about 50 mg to about 2500 mg, such as about any of about 50 mg to about 150 mg, about 150 mg to about 250 mg, about 250 mg to about 350 mg, about 350 mg to about 450 mg, about 450 mg to about 550 mg, about 550 mg to about 650 mg, about 650 mg to about 750 mg, about 750 mg to about 850 mg, about 850 mg to about 950 mg, about 950 mg to about 1050 mg, about 1050 mg to about 1150 mg, about 1150 mg to about 1250 mg, about 1250 mg to about 1350 mg, about 1350 mg to about 1450 mg, about 1450 mg to about 1550 mg, about 1550 mg to about 1650 mg, about 1650 mg to about 1750 mg, about 1750 mg to about 1850 mg, about 1850 mg to about 1950 mg, about 1950 mg to about 2050 mg, about 2050 mg to about 2150 mg, about 2150 mg to about 2250 mg, about 2250 mg to about 2350 mg, or about 2350 mg to about 2450 mg. In some embodiments, the oral drug dosage form has a total weight of at least about 50 mg, such as at least about any of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, or 2500 mg. In some embodiments, the oral drug dosage form has a total weight of less than about 2500 mg, such as less than about any of 2400 mg, 2300 mg, 2200 mg, 2100 mg, 2000 mg, 1900 mg, 1800 mg, 1700 mg, 1600 mg, 1500 mg, 1400 mg, 1300 mg, 1200 mg, 1100 mg, 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, 100 mg, or 50 mg.

The dosage units described herein comprise: a first modulated-release (MR1) portion comprising the drug; and a second modulated-release (MR2) portion. In some embodiments, the dosage unit comprises one or more additional modulated-release portions. In some embodiments, the dosage unit comprises one or more other components, such as a shell or an intermediate portion.

For example, in some embodiments, dosage units described herein comprise: an immediate-release (IR) portion, such as an IR layer, comprising the drug and having an immediate-release profile; and an extended release (ER) portion, such as an ER layer, comprising the drug and having an extended-release profile. In some embodiments, the dosage unit further comprises a shell, wherein the IR portion and ER portion are partially surrounded by a shell. In some embodiments, dosage units described herein comprise: a first ER portion, such as an ER layer, comprising the drug and having an extended-release profile; and a second ER portion, such as a second ER layer, comprising the drug and having an extended-release profile. In some embodiments, the dosage unit further comprises a shell, wherein the first ER portion and the second ER portion are partially surrounded by a shell. In some embodiments, the dosage unit further comprises an intermediate portion, such as an intermediate layer. In some embodiments, the dosage unit comprises another drug.

The dosage units described herein may comprise a variety of combinations of the components thereof (e.g., one or more IR portions, one or more ER portions, one or more intermediate portions, and a shell), and may be arranged in a diverse array of configurations. The orientation of components (such as an IR portion, an ER portion, an intermediate portion, a shell) of a dosage unit may be assembled in a multitude of orientations relative to one another. In some embodiments, to facilitate the description of components of a dosage unit within the scope of the present disclosure, the orientation of a first component (such as an IR portion) relative to another component (such as an ER portion) may be described based on use of an imaginary axis that is perpendicular to the eroding surface (e.g., following exposure to GI fluid) of one or more, or all, of an IR portion, an ER portion, and an intermediate portion of the dosage unit. In some embodiments, the eroding surfaces of two components (whether or not erosion occurs concurrently) may overlap, substantially or in part, as assessed along an imaginary perpendicular axis, and the two components may be referred to as stacked. In some embodiments, the eroding surfaces of two components (whether or not erosion occurs concurrently) may not overlap as assessed along an imaginary perpendicular axis, and the two components may be referred to as positioned side-by-side.

As discussed herein, for purposes of brevity, in many embodiments disclosed herein a dosage form comprising an IR portion (as the MR1 portion) and an ER portion (as the MR2 portion) is described to exemplify the invention. This disclosure is not to be understood as limiting the description herein and such teachings can also be applied to other configurations wherein the MR1 portion and/or the MR2 portion are different modulated-release portions.

In some embodiments, the IR portion and the ER portion are in direct contact with each other. In some embodiments, the IR portion and the ER portion are separated, in whole or in part, by another component, e.g., a shell and/or an intermediate portion. In some embodiments, the IR portion and the ER portion of a dosage unit are stacked on top of each other. In some embodiments, the IR portion and the ER portion of a dosage unit are positioned side-by-side.

In some embodiments, the JR portion and the ER portion are partially surrounded by a shell. In some embodiments, the shell is in direct contact with the IR portion and the ER portion. In some embodiments, the shell has a slower dissolution rate than the ER portion. In some embodiments, the shell is not in direct contact with the IR portion and/or the ER portion. In some embodiments, the shell is in direct contact with an intermediate portion.

In some embodiments, the dosage unit comprises an IR portion, an ER portion, and a shell, wherein the IR portion has a top surface and a bottom surface, wherein the ER portion has a top surface and a bottom surface, and wherein the shell is in direct contact with both the IR portion and the ER portion and leaves one surface of the IR portion and/or one surface of the ER portion exposed. In some embodiments, the IR portion is stacked on top of the ER portion, wherein the shell leaves only the top surface of the IR portion exposed. In some embodiments, the bottom surface of the IR portion is in direct contact with the top surface of the ER portion. In some embodiments, the bottom surface of the ER portion is in direct contact with the shell. In some embodiments, the IR portion is stacked on top of the ER portion, wherein the shell leaves the top surface of the IR portion exposed and the bottom surface of the ER portion exposed. In some embodiment, the dosage unit further comprises an intermediate layer, wherein the intermediate portion is position between the IR portion and ER portion.

In some embodiments, the dosage unit further comprises a second IR portion, wherein the second IR portion has a top surface and a bottom surface, wherein the ER portion is stacked on top of the second IR portion, and wherein the shell leaves only the top surface of the IR portion exposed. In some embodiments, the bottom surface of the ER portion is in direct contact with the top surface of the second IR portion. In some embodiments, the bottom surface of the second IR portion is in direct contact with the shell.

In some embodiments, the ER portion is stacked on top of the IR portion, wherein the shell leaves only the top surface of the ER portion exposed. In some embodiments, the bottom surface of the ER portion is in direct contact with the top surface of the IR portion. In some embodiments, the bottom surface of the IR portion is in direct contact with the shell.

In some embodiments, the IR portion is stacked on top of the ER portion, wherein the shell leaves the top surface of the IR portion and the bottom surface of the ER portion exposed. In some embodiments, the bottom surface of the IR portion is in direct contact with the top surface of the ER portion. In some embodiments, wherein the shell leaves the top surface of the IR portion and the bottom surface of the ER portion exposed, wherein the dosage unit further comprises an intermediate portion, wherein the intermediate layer is between the IR portion and the ER portion. In some embodiments, wherein the shell leaves the top surface of the IR portion and the bottom surface of the ER portion exposed, and wherein a portion of the shell is positioned between the IR portion and the ER portion.

In some embodiments, the dosage unit further comprises an intermediate portion. In some embodiments, the intermediate portion is between the IR portion and the ER portion. In some embodiments, the intermediate portion is in direct contact with the IR portion and the ER portion. In some embodiments, the intermediate portion is only in direct contact with the IR portion. In some embodiments, the intermediate portion is only in direct contact with the ER portion. In some embodiments, the intermediate portion is stacked on top of the ER portion. In some embodiments, the intermediate portion is stacked on top of the IR portion.

In some embodiments, the IR portion and the ER portion of a dosage unit are positioned side-by-side with each other. In some embodiments, the IR portion and the ER portion are positioned side-by-side with each other and partially surrounded by a shell (e.g., in direct contact with a shell), wherein the shell leaves the top surface of both the IR layer and the ER layer exposed. In some embodiments, the bottom surface of both the JR portion and the ER portion are in direct contact with the shell. In some embodiments, the dosage unit further comprises an intermediate layer, wherein the intermediate layer is between the IR portion and the ER portion. In some embodiments, the IR portion and the ER portion are positioned side-by-side with each other, wherein the shell leaves the top surface of both the IR portion and the ER portion exposed, and wherein the shell leaves the bottom surface of both the IR portion and the ER portion exposed. In some embodiments, the IR portion and the ER portion are positioned side-by-side with each other, wherein the shell leaves the top surface of both the IR portion and the ER portion exposed, and wherein a portion of the shell is positioned between the IR portion and the ER portion.

In some embodiments, the IR portion surrounds, in whole or in part, the ER portion. In some embodiments, the ER portion surrounds, in whole or in part, the IR portion. In some embodiments, the IR portion and/or the ER portion surround, in whole or in part, another component of the dosage unit, such as an intermediate portion or a void, such as a gas-filled void. In some embodiments, at least a portion of the IR portion is in direct contact with a portion the ER portion. In some embodiments, at least a portion of the ER portion is in direct contact with a portion the IR portion. In some embodiments, the IR portion and ER portion are not in direct contact.

In some embodiments, the IR portion and the ER portion of the dosage unit are configured in a concentric-style configuration. In some embodiments, other components of the dosage unit are also configured in a concentric-style configuration, such as an intermediate portion, a shell, or a void. In some embodiments, at least a portion of the IR portion is in direct contact with a portion the ER portion. In some embodiments, at least a portion of the ER portion is in direct contact with a portion the IR portion. In some embodiments, the IR portion and ER portion are not in direct contact

In some embodiments, the dosage unit comprises more than one IR portions, such as any of 2, 3, 4, 5, or 6 IR portions, e.g., layers. In some embodiments, the dosage unit comprises more than one ER portions, such as any of 2, 3, 4, 5, or 6 ER portions. e.g., layers. In some embodiments, the dosage unit comprises one or more intermediate portions, such as any of 2, 3, 4, 5, or 6 intermediate portions, e.g., layers. In some embodiments, layer, when used in reference to an IR layer, an ER layer, or an intermediate layer, refers to the configuration of a component of the dosage unit and each may comprise a plurality of printed layers of the same material. In some embodiments, the portion or layer has a fill density, such a three-dimensional printed fill density. In some embodiments, the components described herein, e.g., the IR portion, the ER portion, the intermediate portion, and the shell, each comprise a plural of printed layers. In some embodiments, the plurality of printed layers is between about 5 printed layers to about 2500 printed layers, such as between any of about 10 printed layers to about 2500 printed layers, about 25 printed layers to about 100 printed layers, about 50 printed layers to about 200 printed layers, about 100 printed layers to about 200 printed layers, about 150 printed layers to about 250 printed layers, about 200 printed layers to about 250 printed layers, about 500 printed layers to about 1000 printed layers, or about 2000 printed layers to about 2400 printed layers. In some embodiments, the thickness of a printed layer is no more than about 5 mm, such as no more than about any of 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm. In some embodiments, the thickness of a printed layer is about any of 5 mm, 4 mm, 3 mm, 2 mm, 1 mm, 0.9 mm, 0.8 mm, 0.7 mm, 0.6 mm, 0.5 mm, 0.4 mm, 0.3 mm, 0.2 mm, 0.1 mm, 0.09 mm, 0.08 mm, 0.07 mm, 0.06 mm, 0.05 mm, 0.04 mm, 0.03 mm, 0.02 mm, or 0.01 mm.

In some embodiments, the total amount of a drug contained in a dosage unit is divided between an IR portion and an ER portion in any desired ratio. In some embodiments, a portion of the total amount of a drug contained in a dosage unit is divided between an IR portion and an ER portion in any desired ratio. In some embodiments, the IR:ER drug ratio is between about 1:100 and about 100:1. In some embodiments, the IR:ER drug ratio is about any of 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. In some embodiments, the drug ratio between an IR portion and an ER portion is between about 1:100 and about 100:1. In some embodiments, the drug ratio between an IR portion and an ER portion is about any of 10:1, 9:1, 8:1, 7:1, 6:1, 5:1, 4:1, 3:1, 2:1, 1:1, 1:2, 1:3, 1:4.1:5, 1:6, 1:7, 1:8, 1:9, or 1:10.

The dosage units described herein may be any shape or size suitable for oral administration. In some embodiments, at least a portion of the dosage unit, such as size and/or shape, is based on an interaction with one or more other dosage units. For example, in some embodiments, the shape of the bottom of a dosage unit, such as the shape of a shell, matches the shape of the of the bottom of another dosage unit, wherein the bottoms of the two dosage units associate with one another, such as are in direct contact with one another. In some embodiments, the shape of the dosage unit comprises a cylinder, oval, bullet shape, arrow head shape, triangle, arced triangle, square, arced square, rectangle, arced rectangle, diamond, pentagon, hexagon, octagon, half moon, almond, or a combination thereof.

In some embodiments, the largest dimension, e.g., largest diameter, of the dosage unit is about 1 mm to about 25 mm, such as any of about 2 mm to about 10 mm, about 5 mm to about 12 mm, about 8 mm to about 15 mm, about 5 mm to about 10 mm, or about 7 mm to about 9 mm. In some embodiments, the largest dimension, e.g., largest diameter, of the dosage unit is less than about 25 mm, such as less than about any of 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the largest dimension, e.g., largest diameter, of the dosage unit is greater than about 1 mm, such as greater than about any of 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm. In some embodiments, the largest dimension crossing an oral drug dosage form, e.g., largest diameter, is about any of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm.

In some embodiments, the dosage unit has a thickness of about 1 mm to about 25 mm, such as any of about 2 mm to about 10 mm, about 5 mm to about 12 mm, about 8 mm to about 15 mm, about 5 mm to about 10 mm, or about 7 mm to about 9 mm. In some embodiments, the dosage unit has a thickness of less than about 25 mm, such as less than about any of 24 mm, 23 mm, 22 mm, 21 mm, 20 mm, 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, or 1 mm. In some embodiments, the dosage unit has a thickness of greater than about 1 mm, such as greater than about any of 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm. In some embodiments, the dosage unit has a thickness of about any of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm.

In some embodiments, the total weight of the dosage unit is about 20 mg to about 1500 mg, such as about any of about 50 mg to about 150 mg, about 150 mg to about 250 mg, about 160 mg to about 170 mg, about 250 mg to about 350 mg, about 350 mg to about 450 mg, about 450 mg to about 550 mg, about 550 mg to about 650 mg, about 650 mg to about 750 mg, about 750 mg to about 850 mg, about 850 mg to about 950 mg, about 950 mg to about 1050 mg, about 1050 mg to about 1150 mg, about 1150 mg to about 1250 mg, about 1250 mg to about 1350 mg, or about 1350 mg to about 1450 mg. In some embodiments, the total weight of the dosage unit is less than about 1500 mg, such as less than about any of 1450 mg, 1400 mg, 1350 mg, 1300 mg, 1250 mg, 1200 mg, 1150 mg, 1100 mg, 1050 mg, 1000 mg, 950 mg, 900 mg, 850 mg, 800 mg, 750 mg, 700 mg, 650 mg, 600 mg, 550 mg, 500 mg, 475 mg, 450 mg, 425 mg, 400 mg, 375 mg, 350 mg, 325 mg, 300 mg, 275 mg, 250 mg, 225 mg, 200 mg, 175 mg, 150 mg, 125 mg, 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, or 25 mg. In some embodiments, the total weight of the dosage unit is greater than about 20 mg, such as greater than about any of 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, or 1450 mg. In some embodiments, the total weight of the dosage unit is about any of 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 160 mg, 165 mg, 170 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, or 1450 mg.

In some embodiments, one or more dosage units, such as 2, 3, or 4 dosage units, described herein can be configured to form an oral drug dosage form, wherein each dosage unit comprises an IR portion, such as an IR layer, and an ER portion, such as an ER layer, and optionally an intermediate portion, such as an intermediate layer, and/or a shell. In some embodiments, the dosage units of an oral drug dosage form are the same.

In some embodiments, the oral drug dosage form comprising a fixed amount of a drug formulated and configured to have a desired composite pharmacokinetic (PK) profile, comprises two dosage units stacked back-to-back. In some embodiments, each dosage unit comprises: an immediate-release (IR) layer comprising the drug, the IR layer having an immediate-release profile; an extended release (ER) layer comprising the drug, the ER layer having an extended-release profile; and a shell, wherein the IR layer has a top surface and a bottom surface, wherein the ER layer has a top surface and a bottom surface, wherein the shell partially surrounds the IR layer and the ER layer, and wherein the shell is in direct contact with both the IR layer and the ER layer and leaves one surface of the IR layer and/or one surface of the ER layer exposed. In some embodiments, each dosage unit comprises: a first ER layer comprising the drug, the first ER layer having an extended-release profile; a second ER layer comprising the drug, the second ER layer having an extended-release profile; and a shell, wherein the first ER layer has a top surface and a bottom surface, wherein the second ER layer has a top surface and a bottom surface, wherein the shell partially surrounds the first ER layer and the second ER layer, and wherein the shell is in direct contact with both the first ER layer and the second ER layer and leaves one surface, e.g., the top surface or the bottom surface, of the first ER layer and/or one surface, e.g., the top surface or the bottom surface, of the second ER layer exposed. In some embodiments, the drug has linear pharmacokinetics. In some embodiments, the shell is non-erodible.

In some embodiments, in at least one of the dosage units of an oral drug dosage form, the IR layer and the ER layer are stacked on top of each other. In some embodiments, in at least in one of the dosage units of an oral drug dosage form, the IR layer is stacked on top of the ER layer, the shell leaves only the top surface of the IR layer exposed. In some embodiments, the bottom surface of the IR layer is in direct contact with the top surface of the ER layer. In some embodiments, in at least one of the dosage units of an oral drug dosage form, the dosage unit further comprises a second IR layer, wherein the second IR layer has a top surface and a bottom surface, wherein the ER layer is stacked on top of the second IR layer, and wherein the shell leaves only the top surface of the IR layer exposed. In some embodiments, the bottom surface of the ER layer is in direct contact with the top surface of the second IR layer. In some embodiments, in at least one of the dosage units of an oral drug dosage form, the ER layer is stacked on top of the IR layer, and wherein the shell leaves only the top surface of the ER layer exposed. In some embodiments, the bottom surface of the ER layer is in direct contact with the top surface of the IR layer.

In some embodiments, in at least one of the dosage units of an oral drug dosage form, the IR layer and the ER layer are positioned side-by-side with each other. In some embodiments, in at least one of the dosage units of an oral drug dosage form, the IR layer and the ER layer are positioned side-by-side with each other, wherein the shell leaves the top surface of both the IR layer and the ER layer exposed. In some embodiments, in at least one of the dosage units of an oral drug dosage form, the dosage unit further comprises a second 1R layer, wherein the second IR layer has a top surface and a bottom surface, wherein the IR layer and the ER layer is stacked on top of the second IR layer, and wherein the shell leaves the top surface of the IR layer and the ER layer exposed. In some embodiments, the bottom surface of the IR layer and the ER layer are in direct contact with the top surface of the second IR layer. In some embodiments, in at least one of the dosage units of an oral drug dosage form, the dosage unit further comprises an intermediate layer, wherein the intermediate later is positioned between the IR layer and the ER layer.

In some embodiments, in at least one of the dosage units of an oral drug dosage form, the first ER portion, such as first ER layer, and the second ER portion, such as second ER layer, are positioned side-by-side with each other. In some embodiments, in at least one of the dosage units of an oral drug dosage form, the first ER portion and the second ER portion are positioned side-by-side with each other, wherein the shell leaves the top surface of both the first ER portion and the second ER portion exposed. In some embodiments, in at least one of the dosage units of an oral drug dosage form, the dosage unit further comprises an IR portion. In some embodiments, in at least one of the dosage unit of an oral drug dosage form, the shell separates the first ER portion and the second ER portion. In some embodiments, in at least one of the dosage units of an oral drug dosage form, the dosage unit further comprises an intermediate layer, wherein the intermediate later is positioned between the first ER portion and the second ER portion.

In some embodiments, wherein the oral drug dosage form comprises two dosage units stacked back-to-back, the two dosage units are the same. In some embodiments, wherein the oral drug dosage form comprises two dosage units stacked back-to-back, the two dosage units are different. In some embodiments, wherein the oral drug dosage form comprises two dosage units positioned side-by-side, the two dosage units are the same. In some embodiments, wherein the oral drug dosage form comprises two dosage units positioned side-by-side, the two dosage units are different. In some embodiments, substantially all of the IR portion, such as IR layer, erodes within at least about 20 minutes following administration of the oral drug dosage form to an individual. In some embodiments, the IR portion, such as IR layer, comprises an erodible material. In some embodiments, the ER portion, such as ER portion, comprises an erodible material, and wherein the drug contained in the ER portion is released from the oral drug dosage form over a period of at least about 6 hours.

In some embodiments, the components of the dosage units and/or the oral drug dosage forms described herein, such as the IR portion, the ER portion, the intermediate portion, and the shell, are integrated (e.g., do not form components that may be readily separated).

In some embodiments, the dosage unit and/or the oral drug dosage form comprises a coating, such as an outer coating. In some embodiments, the outer coating is a sugar coating. In some embodiments, the outer coating is a cosmetic coating. In some embodiments, the outer coating is a film coating. In some embodiments, the outer coating is a polymer coating.

Certain configurations and aspects of the components of the dosage unit are exemplified herein. One of ordinary skill in the art will understand that, in view of the disclosure provided herein, the exemplified configurations do not limit the scope of the oral drug dosage forms having a fixed amount of a drug and a desired composite pharmacokinetic (PK) profile in an individual provided herein. Some aspects of the oral drug dosage forms are described herein in a modular fashion, and such aspects may be combined to obtained oral drug dosage forms envisioned within the scope of the present application.

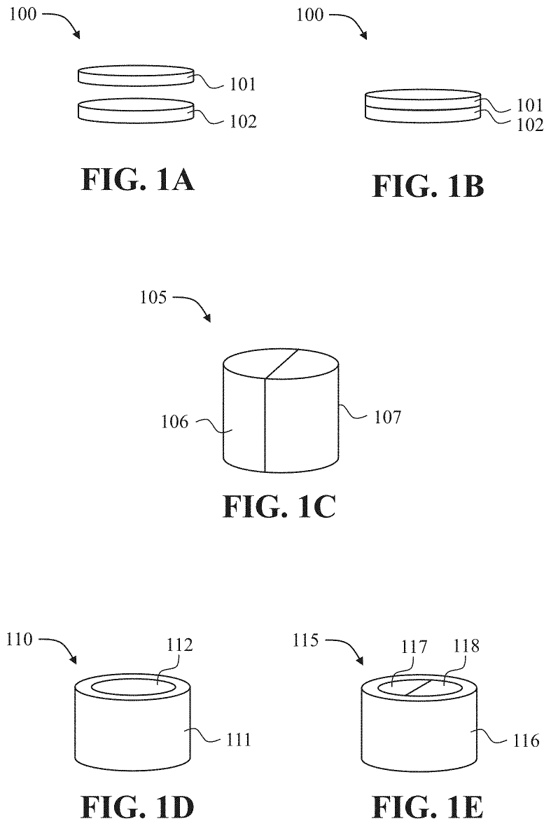

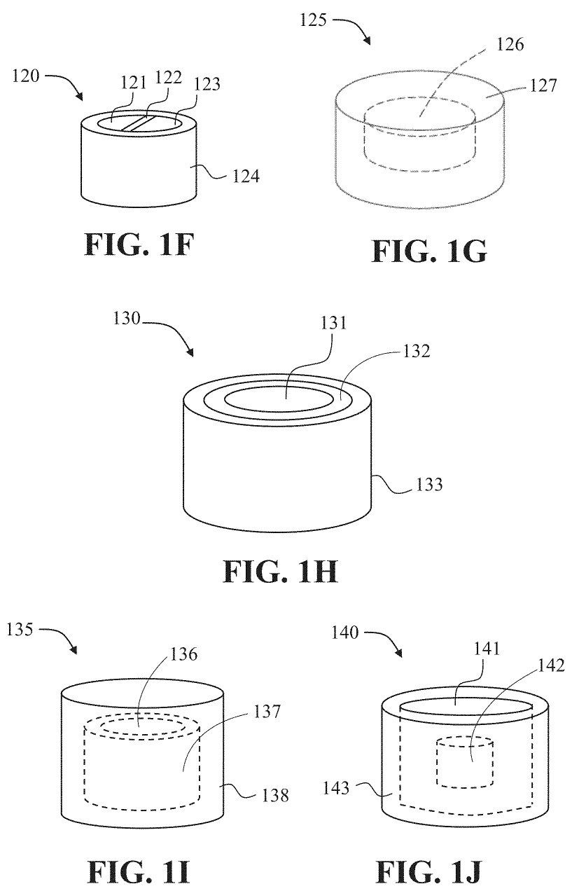

For purposes of example and explanation of the disclosure herein, exemplary oral drug dosage forms, are illustrated in FIG. 1. As shown in FIGS. 1A and B, in some embodiment, the oral drug dosage form comprises a dosage unit 100 comprising: an IR portion, such as an IR layer 101; and an ER portion, such as an ER layer 102, wherein the IR portion 101 and the ER portion 102 are stacked on top of each other. FIG. 1A shows a deconstructed view of the IR portion 101 and the ER portion 102. FIG. 1B shows a constructed view of the IR portion 101 and the ER portion 102.

As shown in FIG. 1C, in some embodiments, the oral drug dosage form comprises a dosage unit 105 comprising: an IR portion 106; and an ER portion 107, wherein the IR portion 106 and the ER portion 107 are positioned side-by-side with each other.

As shown in FIG. 1D, in some embodiments, the oral drug dosage form comprises a dosage unit 110 comprising: an IR portion, such as an IR layer 112; and an ER portion, such as an ER layer, wherein the IR portion 112 and the ER portion are stacked on top of each other, and wherein the IR portion 112 and ER portion are partially surrounded by a shell 111. As shown in FIG. 1E, in some embodiments, the oral drug dosage form comprises a dosage unit 115 comprising: an IR portion 117; and an ER portion 118, wherein the IR portion 117 and the ER portion 118 are positioned side-by-side with each other, and wherein the IR portion 117 and ER layer 118 are partially surrounded by a shell 116. As shown in FIG. 1F, in some embodiments, the oral drug dosage form comprises a dosage unit 120 comprising: an IR portion 121; and an ER portion 123, wherein the IR portion 121 and the ER portion 123 are positioned side-by-side with each other, wherein the IR portion 121 and the ER portion 123 are separated by a component, such as a shell or intermediate portion 122, and wherein the IR portion 121 and ER layer 123 are partially surrounded by a shell 124.

As shown in FIG. 1G, in some embodiments, the oral drug dosage form comprises a dosage unit 125 comprising a component that surrounds another component, e.g., (i) an IR portion 126 surrounded by an ER portion 127, or (ii) an ER portion 126 surrounded by an IR portion 127.

As shown in FIG. 1H in some embodiments, the oral drug dosage form comprises a dosage unit 130 comprising components in a concentric-style configuration, e.g., (i) an IR portion 131 partially surrounded by an ER portion 132 which is partially surround by a shell 133, (ii) an ER portion 131 partially surrounded by an IR portion 132 which is partially surround by a shell 133, (iii) an IR portion 132 partially surrounded by an ER portion 133, wherein the IR portion partially surrounds a core, such as an intermediate portion 131, (iv) an IR portion 132 partially surrounded by an ER portion 133, wherein the dosage unit comprises a void 131, (v) an ER portion 132 partially surrounded by an IR portion 133, wherein the ER portion partially surrounds a core, such as an intermediate portion 131, (vi) an ER portion 132 partially surrounded by an IR portion 133, wherein the dosage unit comprises a void 131, or (vii) an IR portion 131 partially surrounded by an intermediate portion 132 which is partially surrounded by an ER portion 133.

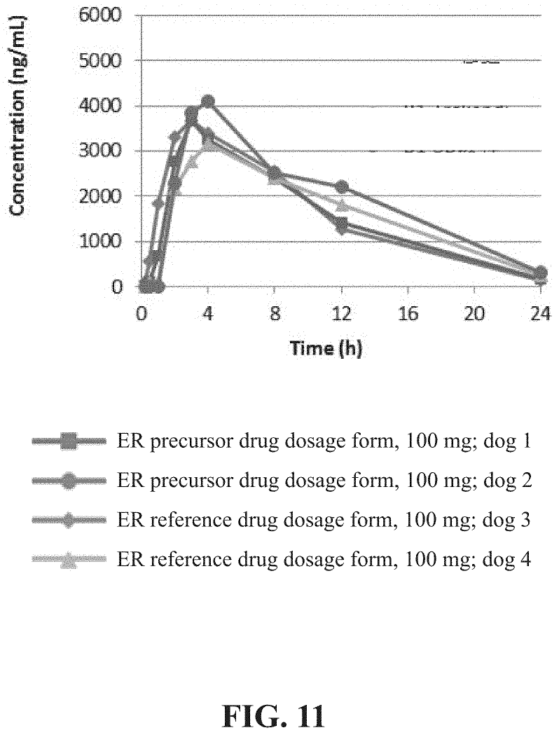

As shown in FIG. 11, in some embodiments, the oral drug dosage form comprises a dosage unit 135 comprising a component that surrounds one or more other components, e.g., (i) an IR portion 136 partially surrounded by an ER portion 137 which is surround by an IR portion or an intermediate portion 138, (ii) an ER portion 136 partially surrounded by an IR portion or an intermediate portion 137 which is surround by an IR portion or an intermediate portion 138, (iii) an JR portion 137 surrounded by an ER portion 138, wherein the IR portion partially surrounds a core, such as an intermediate portion 136, (iv) an IR portion 137 surrounded by an ER portion 138, wherein the dosage unit comprises a void 136, (v) an ER portion 137 surrounded by an IR portion 138, wherein the ER portion partially surrounds a core, such as an intermediate portion 136, (vi) an ER portion 137 surrounded by an IR portion 138, wherein the dosage unit comprises a void 136, or (vii) an IR portion 136 partially surrounded by an intermediate portion 137 which is surrounded by an ER portion 138.

As shown in FIG. 1J, in some embodiments, the oral drug dosage form comprises a dosage unit 140 comprising: (i) an ER portion 142 surrounded by an IR portion 141 which is partially surrounded by a shell 143, or (ii) an ER portion 142 surrounded by an intermediate portion 141 which is partially surrounded by an ER portion, an intermediate portion, or a shell 143.

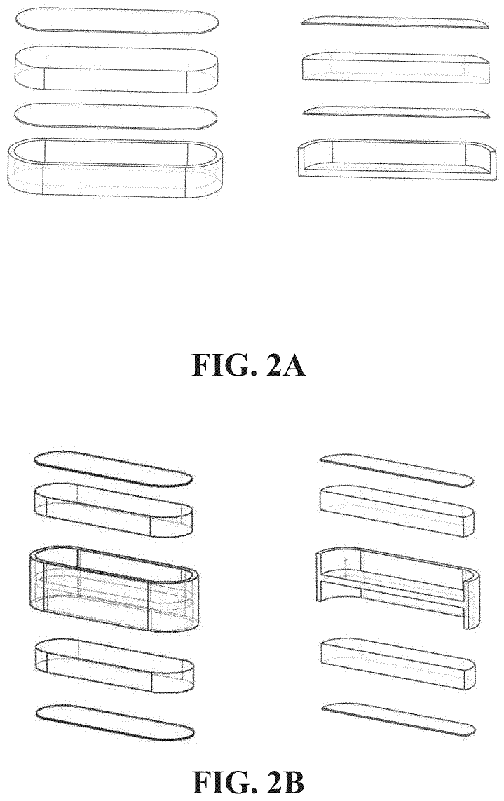

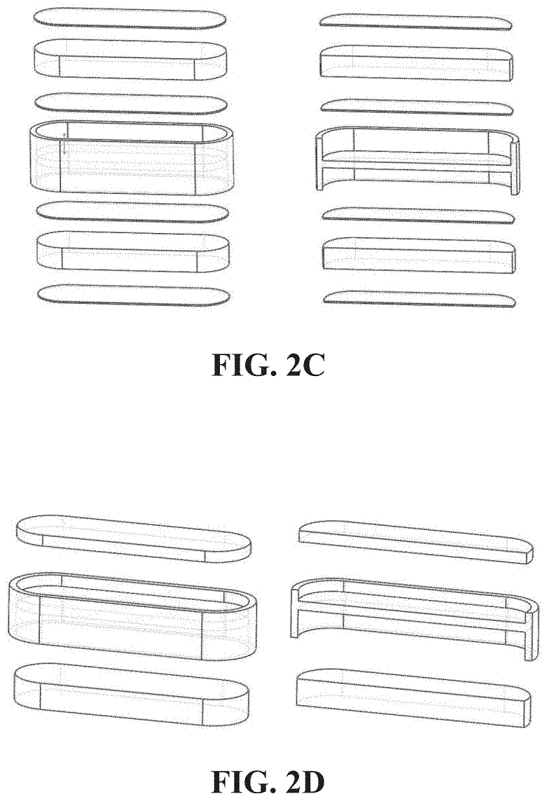

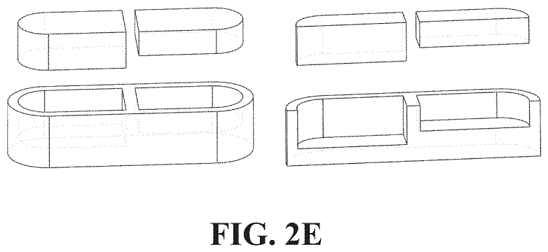

Additional multi-portion oral drug dosage forms and dosage units are contemplated herein. For example, as shown in FIGS. 2A-2E, in some embodiments, the oral drug dosage form comprises more than one ER portion, such as an ER layer, and/or more than one IR portion, such as an JR layer.

As shown in FIG. 2D, the oral drug dosage form comprises a dosage unit comprising a MR1 portion and a MR2 portion, wherein a shell or an intermediate portion separates the two modulated-release portions. In some embodiments, the oral drug dosage form or dosage unit comprises an ER portion, such as an ER layer, and an JR portion, such as an JR layer, or a second ER portion, such as an ER layer, wherein the two modulated-release portions are separated by a portion of a shell or an intermediate portion. See, e.g., FIG. 2D and FIG. 2E.

### IR Portions

The IR portions, such as IR layers, described herein comprise a drug and have an immediate release profile. In some embodiments, at least about 60%, such as at least about any of 65%, 70%, 75%, 80%, 85%, 90%, 95%, 96%, 97%, 98%, or 99%, of the drug in an IR portion is released from a dosage unit within about 60 minutes, such as any of about 50 minutes, 40 minutes, 30 minutes, 20 minutes, 10 minutes, or 5 minutes, following administration of the dosage unit to an individual. In some embodiments, at least about 80%, such as at least about any of 85%, 90%, 95%, 96%, 97%, 98%, or 99%, of the drug in an IR portion is released from a dosage unit within about 60 minutes, such as any of about 50 minutes, 40 minutes, 30 minutes, 20 minutes, 10 minutes, or 5 minutes, following administration of the dosage unit to an individual. In some embodiments, at least about 60%, such as at least about any of 65%, 70%, 75%, 80%, 85%, 90%, 95%, 96%, 97%, 98%, or 99%, of the IR portion erodes within about 60 minutes, such as any of about 50 minutes, 40 minutes, 30 minutes, 20 minutes, 10 minutes, or 5 minutes, following administration of a dosage unit to an individual. In some embodiments, at least about 80%, such as at least about any of 85%, 90%, 95%, 96%, 97%, 98%, or 99%, of the IR layer erodes within about 60 minutes, such as any of about 50 minutes, 40 minutes, 30 minutes, 20 minutes, 10 minutes, or 5 minutes, following administration of a dosage unit to an individual.

In some embodiments, the IR portion, such as IR layer, has a drug mass fraction (mass_{drug}/mass_{layer}) of between about 0.05 to about 1, such as any of about 0.1 to about 0.5, about 0.2 to about 0.6, about 0.3 to about 0.7, about 0.4 to about 0.8, about 0.5 to about 0.9, about 0.5 to about 1. In some embodiments, the IR portion has a drug mass fraction of at least about 0.05, such as at least about any of 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1. In some embodiments, the IR portion has a drug mass fraction of 1 or less, such as any of 0.95 or less, 0.9 or less, 0.85 or less, 0.8 or less, 0.75 or less, 0.7 or less, 0.65 or less, 0.6 or less, 0.55 or less, 0.5 or less, 0.45 or less, 0.4 or less, 0.35 or less, 0.3 or less, 0.25 or less, 0.2 or less, 0.15 or less, 0.1 or less, or 0.05 or less.

In some embodiments, the JR portion, such as JR layer, comprises at least about 0.001% drug, such as at least about any of 0.005% drug, 0.01% drug, 0.05% drug, 0.1% drug, 0.5% drug, 1% drug, 2% drug, 3% drug, 4% drug, or 5% drug. In some embodiments, the IR portion, such as IR layer, comprises between about 0.001% and 100% drug.

The IR portion(s) of the oral drug dosage forms described herein may comprise any amount of a drug. As described herein, the amount of a drug in an IR layer may depend on, e.g., the total amount of drug in the oral drug dosage form, the desired release profile, and the desired PK profile. In some embodiments, the amount of a drug in an IR layer is at about 0.001 mg to about 2000 mg. In some embodiments, the amount of a drug in an IR layer is at least about 0.001 mg, such as at least about any of 0.01 mg, 0.1 mg, 0.25 mg, 0.5 mg, 0.75 mg, 1 mg, 2 mg, 3 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 600 mg, 700 mg, 800 mg, 900 mg, 1000 mg, 1250 mg, or 1500 mg.

In some embodiments, the IR layer further comprises at least one other component, such as 2, 3, 4, 5, or 6 other components. In some embodiments, the IR layer comprises at least one other component admixed with a drug. In some embodiments, the IR layer further comprises a structural material. In some embodiments, the IR layer further comprises a material, such as a filler, a binder, a controlled-release polymer, a lubricant, a glidant, a disintegrant, a thermoplastic material, or a plasticizer.

In some embodiments, IR portion further comprises an excipient. In some embodiments, the excipient is selected from the group consisting of: acacia, alginate, alginic acid, aluminum acetate, benzyl alcohol, butyl paraben, butylated hydroxy toluene, citric acid, calcium carbonate, candelilla wax, croscarmellose sodium, confectioner sugar, colloidal silicone dioxide, cellulose, plain or anhydrous calcium phosphate, camuba wax, corn starch, carboxymethylcellulose calcium, calcium stearate, calcium disodium ethylenediaminetetraacetic acid (EDTA), copolyvidone, castor oil hydrogenated, calcium hydrogen phosphate dehydrate, cetylpyridine chloride, cysteine HCl, crosspovidone, calcium phosphate dibasic, calcium phosphate tribasic, dibasic calcium phosphate, disodium hydrogen phosphate, dimethicone, erythrosine sodium, ethyl cellulose, ethylenediaminetetraacetic acid (EDTA), gelatin, glyceryl monooleate, glycerin, glyceryl monostearate, glyceryl behenate, hydroxy propyl cellulose, hydroxyl propyl methyl cellulose, hypromellose, hydroxypropyl methylcellulose (HPMC) phthalate, iron oxide, ferric oxide, iron oxide yellow, iron oxide red, lactose (hydrous, anhydrous, monohydrate, or spray dried), magnesium stearate, microcrystalline cellulose, mannitol, methyl cellulose, magnesium carbonate, mineral oil, methacrylic acid copolymer, magnesium oxide, methyl paraben, polyvinylpyrrolidone (PVP), polyethylene glycol (PEG), polysorbate 80, propylene glycol, polyethylene oxide, propylene paraben, polaxamer, polaxamer 407, polaxamer 188, potassium bicarbonate, potassium sorbate, potato starch, phosphoric acid, polyoxy 140 stearate, sodium starch glycolate, starch pregelatinized, sodium crossmellose, sodium lauryl sulfate, starch, silicon dioxide, sodium benzoate, stearic acid, sucrose, sorbic acid, sodium carbonate, saccharin sodium, sodium alginate, silica gel, sorbiton monooleate, sodium stearyl fumarate, sodium chloride, sodium metabisulfite, sodium citrate dehydrate, sodium starch, sodium carboxy methyl cellulose, succinic acid, sodium propionate, titanium dioxide, talc, triacetin, and triethyl citrate.

In some embodiments, IR portion further comprises an erodible material, such as an immediate release material. In some embodiments, the immediate release material is selected from the group consisting of polyvinyl caprolactam-polyvinyl acetate-polyethylene glycol graft copolymer 57/30/13, polyvinylpyrrolidone-co-vinyl-acetate (PVP-VA), polyvinylpyrrolidone-polyvinyl acetate copolymer (PVP-VA) 60/40, polyvinylpyrrolidone (PVP), polyvinyl acetate (PVAc) and polyvinylpyrrolidone (PVP) 80/20, vinylpyrrolidone-vinyl acetate copolymer (VA64) polyethylene glycol-polyvinyl alcohol graft copolymer 25/75, kollicoat IR-polyvinyl alcohol 60/40, polyvinyl alcohol (PVA or PV-OH), poly(ethylene oxide) (PEO), poly(ethylene glycol) (PEG), a cellulose or cellulose derivative, hydroxypropyl methylcellulose acetate succinate or hypromellose acetate succinate (HPMCAS), carbomer, hydroxyl propyl cellulose (HPC), poloxamer, hydroxy propyl methylcellulose phthalate (HPMCP), poloxamer, polyglycolic acid (PGA), a saccharide, glucose, hydrogel, gelatin, sodium alginate, arabic gum, xanthan gum, and a combination thereof.

In some embodiments, IR portion further comprises a release agent. In some embodiments, the release agent is a release rate accelerant, such as lactose, mannitol, or combinations thereof. In some embodiments, the release agent is an excipient. In some embodiments, the release agent is an erodible material.

In some embodiments, IR portion further comprises a thermoplastic material. In some embodiments, the thermoplastic material is admixed with a plasticizer. In some embodiments, the IR portion further comprises a plasticizer. In some embodiments, the plasticizer is triethyl citrate (TEC). In some embodiments, the plasticizer is selected from the group consisting of block copolymers of polyoxyethylene-polyoxypropylene, vitamin e polyethylene glycol succinate, hydroxystearate, polyethylene glycol (such as PEG400), macrogol cetostearyl ether 12, polyoxyl 20 cetostearyl ether, polysorbate 20, polysorbate 60, polysorbate 80, acetin, acetylated triethyl citrate, tributyl citrate, tributyl o-acetylcitrate, triethyl citrate, polyoxyl 15 hydroxystearate, peg-40 hydrogenated castor oil, polyoxyl 35 castor oil, dibutyl sebacate, diethylphthalate, glycerine, methyl 4-hydroxybenzoate, glycerol, castor oil, oleic acid, tryacetin, polyalkylene glycol, and a combination thereof.

In some embodiments, the IR layer is printed via dispensing of an IR material, such as an IR material comprising the components described herein.

### ER Portions

The ER portions, such as ER layers, described herein comprise a drug and have an extended-release profile.

In some embodiments, the drug contained in an ER portion, such as a layer, is released from the dosage unit over a period of time starting from when the ER portion is exposed to GI fluid.

In some embodiments, the drug contained in an ER portion, such as ER layer, is released from a dosage unit over a period of at least about 3 hours, such as at least about any of 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 24 hours, 48 hours, 72 hours, 96 hours, 120 hours, 144 hours, 168 hours, 192 hours, 216 hours, 240 hours, 264 hours, 288 hours, 312 hours, 336 hours, 360 hours, 384 hours, 408 hours, 432 hours, 456 hours, 480 hours, 504 hours, 528 hours, 552 hours, 576 hours, 600 hours, 624 hour, 648 hours, 672 hours, 696 hours, or 720 hours. In some embodiments, the ER portion, such as ER layer, of a dosage unit erodes over a period of at least about 3 hours, such as at least about any of 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 24 hours, 48 hours, 72 hours, 96 hours, 120 hours, 144 hours, 168 hours, 192 hours, 216 hours, 240 hours, 264 hours, 288 hours, 312 hours, 336 hours, 360 hours, 384 hours, 408 hours, 432 hours, 456 hours, 480 hours, 504 hours, 528 hours, 552 hours, 576 hours, 600 hours, 624 hour, 648 hours, 672 hours, 696 hours, or 720 hours.

In some embodiments, the dissolution rate (or erosion rate) of the ER layer is about 0.05 mm/hour to about 0.5 mm/hour. In some embodiments, the dissolution rate (or erosion rate) of the ER layer is at least about 0.05 mm/hour, such as at least about any of 0.1 mm/hour, 0.2 mm/hour, 0.3 mm/hour, 0.4 mm/hour, or 0.5 mm/hour.

In some embodiments, the extended-release profile comprises a zero-order release profile, a first-order release profile, a delayed release profile, a pulsed release profile, an iterative pulsed release profile, or a combination thereof.

In some embodiments, the ER layer has a drug mass fraction (mass_{drug}/mass_{layer}) of between about 0.05 to about 1, such as any of about 0.1 to about 0.5, about 0.2 to about 0.6, about 0.3 to about 0.7, about 0.4 to about 0.8, about 0.5 to about 0.9, about 0.5 to about 1. In some embodiments, the ER layer has a drug mass fraction of at least about 0.05, such as at least about any of 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1. In some embodiments, the ER layer has a drug mass fraction of 1 or less, such as any of 0.95 or less, 0.9 or less, 0.85 or less, 0.8 or less, 0.75 or less, 0.7 or less, 0.65 or less, 0.6 or less, 0.55 or less, 0.5 or less, 0.45 or less, 0.4 or less, 0.35 or less, 0.3 or less, 0.25 or less, 0.2 or less, 0.15 or less, 0.1 or less, or 0.05 or less.

In some embodiments, the ER portion, such as ER layer, comprises at least about 0.001% drug, such as at least about any of 0.005% drug, 0.01% drug, 0.05% drug, 0.1% drug, 0.5% drug, 1% drug, 2% drug, 3% drug, 4% drug, or 5% drug. In some embodiments, the ER portion, such as ER layer, comprises between about 0.001% and 100% drug.

The ER layer(s) of the oral drug dosage forms described herein may comprise any amount of a drug. As described herein, the amount of a drug in an ER layer may depend on, e.g., the total amount of drug in the oral drug dosage form, the desired release profile, and the desired PK profile. In some embodiments, the amount of a drug in an ER layer is at about 0.001 mg to about 2000 mg. In some embodiments, the amount of a drug in an ER layer is at least about 0.001 mg, such as at least about any of 0.01 mg, 0.1 mg, 0.25 mg, 0.5 mg, 0.75 mg, 1 mg, 2 mg, 3 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 600 mg, 700 mg, 800 mg, 900 mg, 1000 mg, 1250 mg, or 1500 mg.

In some embodiments, the drug contained in the ER portion, such as ER layer, is released from a dosage unit at a release rate of between about 0.00001 mg/hour and 500 mg/hour. In some embodiments, the drug contained in the ER portion, such as ER layer, is released from a dosage unit at a release rate of at less than about 500 mg/hour, such as less than about any of 400 mg/hour, 300 mg/hour, 200 mg/hour, 100 mg/hour, 50 mg/hour, 25 mg/hour, 10 mg/hour, 5 mg/hour, or 1 mg/hour.

In some embodiments, the ER portion, such as ER layer, comprises at least one other component. In some embodiments, the ER layer comprises at least one other component admixed with a drug. In some embodiments, the ER layer further comprises a structural material. In some embodiments, the ER layer further comprises a material, such as a filler, a binder, a controlled-release polymer, a lubricant, a glidant, a disintegrant, a thermoplastic material, or a plasticizer.

In some embodiments, the ER layer further comprises an excipient. In some embodiments, the excipient is selected from the group consisting of: acacia, alginate, alginic acid, aluminum acetate, benzyl alcohol, butyl paraben, butylated hydroxy toluene, citric acid, calcium carbonate, candelilla wax, croscarmellose sodium, confectioner sugar, colloidal silicone dioxide, cellulose, plain or anhydrous calcium phosphate, camuba wax, corn starch, carboxymethylcellulose calcium, calcium stearate, calcium disodium ethylenediaminetetraacetic acid (EDTA), copolyvidone, castor oil hydrogenated, calcium hydrogen phosphate dehydrate, cetylpyridine chloride, cysteine HCl, crosspovidone, calcium phosphate dibasic, calcium phosphate tribasic, dibasic calcium phosphate, disodium hydrogen phosphate, dimethicone, erythrosine sodium, ethyl cellulose, ethylenediaminetetraacetic acid (EDTA), gelatin, glyceryl monooleate, glycerin, glyceryl monostearate, glyceryl behenate, hydroxy propyl cellulose, hydroxyl propyl methyl cellulose, hypromellose, hydroxypropyl methylcellulose (HPMC) phthalate, iron oxide, ferric oxide, iron oxide yellow, iron oxide red, lactose (hydrous, anhydrous, monohydrate, or spray dried), magnesium stearate, microcrystalline cellulose, mannitol, methyl cellulose, magnesium carbonate, mineral oil, methacrylic acid copolymer, magnesium oxide, methyl paraben, polyvinylpyrrolidone (PVP), polyethylene glycol (PEG), polysorbate 80, propylene glycol, polyethylene oxide, propylene paraben, polaxamer, polaxamer 407, polaxamer 188, potassium bicarbonate, potassium sorbate, potato starch, phosphoric acid, polyoxy 140 stearate, sodium starch glycolate, starch pregelatinized, sodium crossmellose, sodium lauryl sulfate, starch, silicon dioxide, sodium benzoate, stearic acid, sucrose, sorbic acid, sodium carbonate, saccharin sodium, sodium alginate, silica gel, sorbiton monooleate, sodium stearyl fumarate, sodium chloride, sodium metabisulfite, sodium citrate dehydrate, sodium starch, sodium carboxy methyl cellulose, succinic acid, sodium propionate, titanium dioxide, talc, triacetin, and triethyl citrate.

In some embodiments, the ER layer further comprises an erodible material, such as a sustained release material. In some embodiments, the sustained release material is selected from the group consisting of polyvinyl caprolactam-polyvinyl acetate-polyethylene glycol graft copolymer 57/30/13, polyvinylpyrrolidone-co-vinyl-acetate (PVP-VA), polyvinylpyrrolidone-polyvinyl acetate copolymer (PVP-VA) 60/40, polyvinylpyrrolidone (PVP), polyvinyl acetate (PVAc) and polyvinylpyrrolidone (PVP) 80/20, vinylpyrrolidone-vinyl acetate copolymer (VA64), polyethylene glycol-polyvinyl alcohol graft copolymer 25/75, kollicoat IR-polyvinyl alcohol 60/40, polyvinyl alcohol (PVA or PV-OH), poly(vinyl acetate) (PVAc), an (optionally alkyl-, methyl-, or ethyl-) acrylate, a methacrylate copolymer, an ethacrylate copolymer, poly(butyl methacrylate-co-(2-dimethylaminoethyl) methacrylate-co-methyl methacrylate) 1:2:1, poly(dimethylaminoethylmethacrylate-co-methacrylic esters), poly(ethyl acrylate-co-methyl methacrylate-co-trimethylammonioethyl methacrylate chloride), poly(methyl acrylate-co-methyl methacrylate-co-methacrylic acid) 7:3:1, poly(methacrylic acid-co-methylmethacrylate) 1:2, poly(methacylic acid-co-ethyl acrylate) 1:1, poly(methacylic acid-co-methyl methacrylate) 1:1, poly(ethylene oxide) (PEO), poly(ethylene glycol) (PEG), hyperbranched polyesteramide, a cellulose or cellulose derivative, hydroxypropyl methylcellulose phthalate, hypromellose phthalate, hydroxypropyl methylcellulose or hypromellose (HMPC), hydroxypropyl methylcellulose acetate succinate or hypromellose acetate succinate (HPMCAS), poly(lactide-co-glycolide) (PLGA), carbomer, poly(ethylene-co-vinyl acetate), ethylene-vinyl acetate copolymer, polyethylene (PE), and polycaprolactone (PCL), hydroxyl propyl cellulose (HPC), polyoxyl 40 hydrogenated castor oil, methyl cellulose (MC), ethyl cellulose (EC), poloxamer, hydroxypropyl methylcellulose phthalate (HPMCP), poloxamer, hydrogenated castor and soybean oil, glyceryl palmitostearate, carnauba wax, polylactic acid (PLA), polyglycolic acid (PGA), cellulose acetate butyrate (CAB), colloidal silicon dioxide, a saccharide, glucose, polyvinyl acetate phthalate (PVAP), a wax, beeswax, hydrogel, gelatin, hydrogenated vegetable oil, polyvinyl acetal diethyl aminolactate (AEA), paraffin, shellac, sodium alginate, cellulose acetate phthalate (CAP), fatty oil, arabic gum, xanthan gum, glyceryl monostearate, octadecanoic acid, and a combination thereof.

In some embodiments, the ER layer further comprises a thermoplastic material. In some embodiments, the thermoplastic material is admixed with a plasticizer. In some embodiments, the other component is a plasticizer. In some embodiments, the plasticizer is triethyl citrate (TEC). In some embodiments, the plasticizer is selected from the group consisting of block copolymers of polyoxyethylene-polyoxypropylene, vitamin e polyethylene glycol succinate, hydroxystearate, polyethylene glycol (such as PEG400), macrogol cetostearyl ether 12, polyoxyl 20 cetostearyl ether, polysorbate 20, polysorbate 60, polysorbate 80, acetin, acetylated triethyl citrate, tributyl citrate, tributyl o-acetylcitrate, triethyl citrate, polyoxyl 15 hydroxystearate, peg-40 hydrogenated castor oil, polyoxyl 35 castor oil, dibutyl sebacate, diethylphthalate, glycerine, methyl 4-hydroxybenzoate, glycerol, castor oil, oleic acid, tryacetin, polyalkylene glycol, and a combination thereof.

### Intermediate Portions

In some embodiments, the dosage units described herein further comprise one or more intermediate portions, such as intermediate layers. In some embodiments, the intermediate layer is in direct contact with the IR layer. In some embodiments, the intermediate layer is in direct contact with the ER layer. In some embodiments, the intermediate layer is in direct contact with the shell. In some embodiments, the intermediate layer is in direct contact with the IR layer and the ER layer. In some embodiments, the intermediate layer is in direct contact with the IR layer, the ER layer, and the shell. In some embodiments, the intermediate layer is positioned between the IR layer and the ER layer. In some embodiments, the intermediate layer is positioned between the JR layer and the shell. In some embodiments, the intermediate layer is positioned between the ER layer and the shell.

In some embodiments, the intermediate layer delays release of a drug from a dosage unit. In some embodiments, the intermediate layer delays release of a drug from a dosage unit for about 5 minutes to about 12 hours, such as any of about 5 minutes to about 1 hour, about 1 hours to about 3 hours, about 3 hours to about 6 hours, about 6 hours, to about 9 hours, or about 9 hours to about 12 hours. In some embodiments, the intermediate layer reduces interference of between two or more components that contact the intermediate layer.

In some embodiments, the dissolution rate (or erosion rate) of the intermediate portion is about 0.1 mm/hour to about 50 mm/hour. In some embodiments, the dissolution rate (or erosion rate) of the intermediate portion is at least about 0.1 mm/hour, such as at least about any of 1 mm/hour, 5 mm/hour, 10 mm/hour, 20 mm/hour, 30 mm/hour, or 40 mm/hour. In some embodiments, the dissolution rate (or erosion rate) of the intermediate portion is less than about 50 mm/hour, such as less than about any of 40 mm/hour, 30 mm/hour, 20 mm/hour, 10 mm/hour, 5 mm/hour, or 1 mm/hour.

In some embodiments, the intermediate layer is stack on top of the IR layer, wherein the intermediate layer has a top surface and a bottom surface, wherein the IR layer has a top surface and a bottom surface, and wherein the shell is in direct contact with both the intermediate layer and the IR layer. In some embodiments, the ER layer is stacked on top of the intermediate layer. In some embodiments, the shell leaves the top surface of the intermediate layer exposed.

In some embodiments, the intermediate layer is stack on top of the ER layer, wherein the intermediate layer has a top surface and a bottom surface, wherein the ER layer has a top surface and a bottom surface, and wherein the shell is in direct contact with both the intermediate layer and the ER layer and leaves the top surface of the intermediate layer exposed.

In some embodiments, the intermediate layer is erodible. In some embodiments, the intermediate layer comprises an erodible material. In some embodiments, the intermediate layer is not admixed with the drug. In some embodiments, the intermediate layer is admixed with a different drug. In some embodiments, the intermediate layer blocks interactions of a drug in the IR layer and the ER layer. In some embodiments, the intermediate layer blocks interactions of one or more other components, such as an excipient, in the IR layer and the ER layer. In some embodiments, the intermediate layer blocks migration of a drug and/or one or more other components, such as an excipient, in the IR layer and the ER layer.

In some embodiments, the intermediate layer has a slower dissolution rate than the IR layer. In some embodiments, the intermediate layer has a faster dissolution rate than the IR layer. In some embodiments, the intermediate layer has a slower dissolution rate than the ER layer. In some embodiments, the intermediate layer has a faster dissolution rate than the ER layer. In some embodiments, the intermediate layer has a slower dissolution rate than the IR layer and a faster dissolution rate than the ER layer. In some embodiments, the dissolution rate of the intermediate layer is selected based on a target dissolution rate. In some embodiments, the dissolution rate of the intermediate layer is selected based on a target drug release rate from an oral drug dosage form.

In some embodiments, the intermediate layer doesn't comprise a drug. In some embodiments, the intermediate layer comprises a second drug.

In some embodiments, the intermediate portion, such as intermediate layer, comprises one or more components, such as 2, 3, 4, 5, or 6 components. In some embodiments, the intermediate portion comprises a structural material. In some embodiments, the intermediate portion comprises any one or more of a material, such as a filler, a binder, a controlled-release polymer, a lubricant, a glidant, a disintegrant, a thermoplastic material, or a plasticizer.

In some embodiments, intermediate portion comprises an excipient. In some embodiments, the excipient is selected from the group consisting of: acacia, alginate, alginic acid, aluminum acetate, benzyl alcohol, butyl paraben, butylated hydroxy toluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium stearate, calcium disodium ethylenediaminetetraacetic acid (EDTA), copolyvidone, castor oil hydrogenated, calcium hydrogen phosphate dehydrate, cetylpyridine chloride, cysteine HCl, crosspovidone, calcium phosphate dibasic, calcium phosphate tribasic, dibasic calcium phosphate, disodium hydrogen phosphate, dimethicone, erythrosine sodium, ethyl cellulose, ethylenediaminetetraacetic acid (EDTA), gelatin, glyceryl monooleate, glycerin, glyceryl monostearate, glyceryl behenate, hydroxy propyl cellulose, hydroxyl propyl methyl cellulose, hypromellose, hydroxypropyl methylcellulose (HPMC) phthalate, iron oxide, ferric oxide, iron oxide yellow, iron oxide red, lactose (hydrous, anhydrous, monohydrate, or spray dried), magnesium stearate, microcrystalline cellulose, mannitol, methyl cellulose, magnesium carbonate, mineral oil, methacrylic acid copolymer, magnesium oxide, methyl paraben, polyvinylpyrrolidone (PVP), polyethylene glycol (PEG), polysorbate 80, propylene glycol, polyethylene oxide, propylene paraben, polaxamer, polaxamer 407, polaxamer 188, potassium bicarbonate, potassium sorbate, potato starch, phosphoric acid, polyoxy 140 stearate, sodium starch glycolate, starch pregelatinized, sodium crossmellose, sodium lauryl sulfate, starch, silicon dioxide, sodium benzoate, stearic acid, sucrose, sorbic acid, sodium carbonate, saccharin sodium, sodium alginate, silica gel, sorbiton monooleate, sodium stearyl fumarate, sodium chloride, sodium metabisulfite, sodium citrate dehydrate, sodium starch, sodium carboxy methyl cellulose, succinic acid, sodium propionate, titanium dioxide, talc, triacetin, and triethyl citrate.

In some embodiments, intermediate portion comprises an erodible material. In some embodiments, the erodible material is selected from the group consisting of polyvinyl caprolactam-polyvinyl acetate-polyethylene glycol graft copolymer 57/30/13, polyvinylpyrrolidone-co-vinyl-acetate (PVP-VA), polyvinylpyrrolidone-polyvinyl acetate copolymer (PVP-VA) 60/40, polyvinylpyrrolidone (PVP), polyvinyl acetate (PVAc) and polyvinylpyrrolidone (PVP) 80/20, vinylpyrrolidone-vinyl acetate copolymer (VA64), polyethylene glycol-polyvinyl alcohol graft copolymer 25/75, kollicoat IR-polyvinyl alcohol 60/40, polyvinyl alcohol (PVA or PV-OH), poly(ethylene oxide) (PEO), poly(ethylene glycol) (PEG), a cellulose or cellulose derivative, hydroxypropyl methylcellulose acetate succinate or hypromellose acetate succinate (HPMCAS), carbomer, hydroxyl propyl cellulose (HPC), poloxamer, hydroxypropyl methylcellulose phthalate (HPMCP), poloxamer, polyglycolic acid (PGA), a saccharide, glucose, hydrogel, gelatin, sodium alginate, arabic gum, xanthan gum, and a combination thereof. In some embodiments, the erodible material is selected from the group consisting of polyvinyl caprolactam-polyvinyl acetate-polyethylene glycol graft copolymer 57/30/13, polyvinylpyrrolidone-co-vinyl-acetate (PVP-VA), polyvinylpyrrolidone-polyvinyl acetate copolymer (PVP-VA) 60/40, polyvinylpyrrolidone (PVP), polyvinyl acetate (PVAc) and polyvinylpyrrolidone (PVP) 80/20, vinylpyrrolidone-vinyl acetate copolymer (VA64), polyethylene glycol-polyvinyl alcohol graft copolymer 25/75, kollicoat IR-polyvinyl alcohol 60/40, polyvinyl alcohol (PVA or PV-OH), poly(vinyl acetate) (PVAc), an (optionally alkyl-, methyl-, or ethyl-) acrylate, a methacrylate copolymer, an ethacrylate copolymer, poly(butyl methacrylate-co-(2-dimethylaminoethyl) methacrylate-co-methyl methacrylate) 1:2:1, poly(dimethylaminoethylmethacrylate-co-methacrylic esters), poly(ethyl acrylate-co-methyl methacrylate-co-trimethylammonioethyl methacrylate chloride), poly(methyl acrylate-co-methyl methacrylate-co-methacrylic acid) 7:3:1, poly(methacrylic acid-co-methylmethacrylate) 1:2, poly(methacylic acid-co-ethyl acrylate) 1:1, poly(methacylic acid-co-methyl methacrylate) 1:1, poly(ethylene oxide) (PEO), poly(ethylene glycol) (PEG), hyperbranched polyesteramide, a cellulose or cellulose derivative, hydroxypropyl methylcellulose phthalate, hypromellose phthalate, hydroxypropyl methylcellulose or hypromellose (HMPC), hydroxypropyl methylcellulose acetate succinate or hypromellose acetate succinate (HPMCAS), poly(lactide-co-glycolide) (PLGA), carbomer, poly(ethylene-co-vinyl acetate), ethylene-vinyl acetate copolymer, polyethylene (PE), and polycaprolactone (PCL), hydroxyl propyl cellulose (HPC), polyoxyl 40 hydrogenated castor oil, methyl cellulose (MC), ethyl cellulose (EC), poloxamer, hydroxypropyl methylcellulose phthalate (HPMCP), poloxamer, hydrogenated castor and soybean oil, glyceryl palmitostearate, carnauba wax, polylactic acid (PLA), polyglycolic acid (PGA), cellulose acetate butyrate (CAB), colloidal silicon dioxide, a saccharide, glucose, polyvinyl acetate phthalate (PVAP), a wax, beeswax, hydrogel, gelatin, hydrogenated vegetable oil, polyvinyl acetal diethyl aminolactate (AEA), paraffin, shellac, sodium alginate, cellulose acetate phthalate (CAP), fatty oil, arabic gum, xanthan gum, glyceryl monostearate, octadecanoic acid, and a combination thereof.

In some embodiments, intermediate portion comprises a release agent. In some embodiments, the release agent is a release rate accelerant, such as lactose, mannitol, or combinations thereof. In some embodiments, the release agent is an excipient. In some embodiments, the release agent is an erodible material.

In some embodiments, the intermediate portion comprises a thermoplastic material. In some embodiments, the thermoplastic material is admixed with a plasticizer. In some embodiments, the plasticizer is triethyl citrate (TEC). In some embodiments, the plasticizer is selected from the group consisting of block copolymers of polyoxyethylene-polyoxypropylene, vitamin e polyethylene glycol succinate, hydroxystearate, polyethylene glycol (such as PEG400), macrogol cetostearyl ether 12, polyoxyl 20 cetostearyl ether, polysorbate 20, polysorbate 60, polysorbate 80, acetin, acetylated triethyl citrate, tributyl citrate, tributyl o-acetylcitrate, triethyl citrate, polyoxyl 15 hydroxystearate, peg-40 hydrogenated castor oil, polyoxyl 35 castor oil, dibutyl sebacate, diethylphthalate, glycerine, methyl 4-hydroxybenzoate, glycerol, castor oil, oleic acid, tracetin, polyalkylene glycol, and a combination thereof.

In some embodiments, the intermediate layer is printed via dispensing of an intermediate material, such as an intermediate material comprising the components described herein.

### Shell

In some embodiments, the dosage unit of an oral drug dosage form comprises a shell. In some embodiments, the shell partially surrounds an IR layer and an ER layer of a dosage unit. In some embodiments, the shell partially surrounds an IR layer, an ER layer, and an intermediate layer.

In some embodiments, the shell is not admixed with the drug. In some embodiments, the shell is admixed with a different drug.

In some embodiments, the shell is non-erodible. In some embodiments, the shell has a slower erosion rate than the erosion rate of an ER layer. In some embodiments, the shell does not substantially erode until after substantially all of a drug in an ER layer has been released therefrom. In some embodiments, the shell does not substantially erode for a period of at least about 6 hours, such as at least about any of 7 hours, 8 hours, 9 hours, 10 hours, 11 hours, 12 hours, 14 hours, 16 hours, 18 hours, 20 hours, 22 hours, 24 hours, 30 hours, 36 hours, 48 hours, or 72 hours, after administration of a dosage unit to an individual. In some embodiments, the shell comprises a pH sensitive material, such as a material that erodes in a certain pH range.

In some embodiments, the shell is non-permeable, such as non-permeable to water or gastrointestinal fluid. In some embodiments, the shell is substantially non-permeable.

In some embodiments, the shell comprises a material selected from the group consisting of: EUDRAGIT® RL, EUDRAGIT® RS, polyvinyl acetate and povidone mixtures, methacrylic acid copolymer, aminomethacrylic acid copolymer, methacrylic acid ester copolymer, butyl acrylate, methacrylic acid methylmethacrylate copolymer, ethyl methacrylate-co-methacrylic acid copolymer, butyl acrylate-monobutyl acrylate copolymer, ethyl acrylate-monomethacrylate copolymer, ethyl acrylate-methyl methacrylate copolymer, ethyl acrylate/methyl methacrylate/trimethylaminoethyl methacrylate polymer, methyl cellulose, ethyl cellulose, polyvinyl acetate phthalate, hypromellose succinate, polyethylene glycol-polyvinyl alcohol copolymer, hydroxypropyl methylcellulose phthalate or hypromellose phthalate, polyethylene glycol 15-hydroxystearate, a copolymer of methyl methacrylate and diethylaminoethylmethyl methacrylic acid ester, polymethyl acrylate-polymethyl methacrylate-polymethacrylic acid copolymer, N,N-dimethylaminoethylmethacrylate, polyvinylcaprolactam-polyvinyl acetate-polyethylene glycol graft copolymer, polybutyl methacrylate-poly N,N-dimethylaminoethyl methacrylate-polymethylmethacrylate copolymer, polyvinyl alcohol, polyethylene oxide, polyoxyethylene, hyperbranched polyesteramides, hydroxypropylmethylcellulose or hypromellose, hydroxyethylcellulose, cellulose acetate, vitamin E polyethylene glycol succinate, polydimethylsiloxane alkane, xanthan gum, polylactic acid, polylactide-polylactic acid copolymer, polycaprolactone, carnauba wax, glyceryl palmitostearate, hydrogenated castor oil, cellulose acetate butyrate, polyvinyl acetate, polyethyl acrylate-polymethylmethacrylate-polytrimethylammonium chloride ethyl methacrylate copolymer, polyethylene-polyvinyl acetate copolymer, and chitosan, and a combination thereof.

In some embodiments, the dissolution of the shell is pH dependent. In some embodiments, the dissolution of the shell occurs above a pH of about 5.5 to about 7. In some embodiments, the dissolution of the shell occurs above a pH of about 5.5, about 6, about 6.5, or about 7. In some embodiments, the shell comprises an enteric material.

In some embodiments, the shell is printed via dispensing of a shell material, such as a shell material comprising the components described herein.

### Drugs

The dosage units disclosed herein comprise an IR layer comprising a drug and an ER layer comprising the drug.

In some embodiments, the drug has linear pharmacokinetics or dose-independent pharmacokinetics (the drug exhibits a drug plasma concentration that is directly proportional to the administered dose). In some embodiments, the dosage unit, such as the IR layer and the ER layer, comprises an amount of the drug that is within a linear pharmacokinetic region of the drug. In some embodiments, the dosage unit releases an amount of the drug over time that is within a linear pharmacokinetic region of the drug. Methods of determining if a drug has linear pharmacokinetics or dose-independent pharmacokinetics are known in the art, thus one or ordinary skill in the art can readily assess a drug that in encompassed by the disclosure of the present application directed to linear pharmacokinetic or dose-independent pharmacokinetic drugs. See, e.g., Jeong et al., Biopharm Drug Dispos, 28, 2007.

In some embodiments, the dosage unit comprises one or more additional drugs. In some embodiments, the JR layer comprises one or more additional drugs. In some embodiments, the ER layer comprises one or more additional drugs. In some embodiments, the IR layer comprises an additional drug and the ER layer comprises an additional drug, wherein the additional drug in the IR layer is different than the additional drug in the ER layer. In some embodiments, the IR layer comprises an additional drug and the ER layer comprises an additional drug, wherein the additional drug in the IR layer is the same as the additional drug in the ER layer. In some embodiments, wherein the IR layer and the ER layer comprise an additional drug, the amount of the additional drug may be distributed between the IR layer and the ER layer independently from the amount of drug it the JR layer and the ER layer.

### Desired Composite PK Profiles

The present disclosure provides, in some aspects, methods of designing an oral drug dosage form described herein having a desired composite pharmacokinetic (PK) profile. Generally, pharmacokinetics refers to the movement of a drug in an individual following administration, and may be characterized by, e.g., the time course of drug absorption, bioavailability, blood, serum, and/or plasma drug concentrations over time, drug distribution, drug metabolism, and excretion of the drug.

In some embodiments, the desired composite PK profile of an oral drug dosage form described herein comprises one or more pharmacokinetic parameters. In some embodiments, the pharmacokinetic parameter is a blood, plasma, or serum-based parameter. In some embodiments, the desired composite PK profile comprises one or more pharmacokinetic parameters selected from Cₘₐₓ (e.g., the peak drug concentration in the plasma after administration), tₘₐₓ (the time to reach Cₘₐₓ), area under the curve (AUC; the integral of the concentration-time curve), Cₘᵢₙ (e.g., the lowest (trough) drug concentration in the plasma before the next dose is administered), volume of distribution, elimination half-life, elimination rate constant, and clearance. In some embodiments, the desired composite PK profile comprises Cₘₐₓ and AUC parameters. In some embodiments, the desired composite PK profile comprises Cₘₐₓ, tₘₐₓ and AUC parameters.

In some embodiments, the desired composite PK profile of an oral drug dosage form described herein comprises a range of values for each of the one or more pharmacokinetic parameters, such as any one or more of Cₘₐₓ, tₘₐₓ, and AUC. In some embodiments, the range of values of a pharmacokinetic parameter of a drug is an acceptable threshold, such as an acceptable threshold based on the pharmacokinetic parameter of a reference PK curve of the drug or a desired PK curve of the drug. In some embodiments, the range of values of a pharmacokinetic parameter of a drug is about 60% to about 145%, such as any of about 65% to about 140%, about 70% to about 135%, about 75% to about 130%, about 80% to about 125%, about 85% to about 120%, or about 90% to about 115%, of the pharmacokinetic parameter of a reference PK curve of the drug. In some embodiments, each of the pharmacokinetic parameters of a desired composite PK profile may have the same or a different acceptable threshold. For example, in some embodiments, the desired composite profile comprises more than one pharmacokinetic parameter, wherein one pharmacokinetic parameter has a larger acceptable threshold than another pharmacokinetic parameter.

In some embodiments, the desired composite PK profile is determined based on having a Cₘₐₓ within an acceptable threshold of a reference, such as a reference PK curve or desired PK curve of the drug. In some embodiments, the desired composite PK profile is determined based on having an AUC within an acceptable threshold of a reference, such as a reference PK curve or desired PK curve of the drug. In some embodiments, the desired composite PK profile is determined based on having a tₘₐₓ within an acceptable threshold of a reference, such as a reference PK curve or desired PK curve of the drug. In some embodiments, the desired composite PK profile is determined based on having an AUC and Cₘₐₓ within an acceptable threshold of a reference, such as a reference PK curve or desired PK curve of the drug. In some embodiments, the desired composite PK profile is determined based on having an AUC, Cₘₐₓ, and tₘₐₓ within an acceptable threshold of a reference, such as a reference PK curve or desired PK curve of the drug. In some embodiments, the desired composite PK profile is determined based on having a pharmacokinetic parameter, such as any one or more of AUC, Cₘₐₓ, and tₘₐₓ, within an acceptable threshold of a reference PK curve of the drug, wherein the acceptable threshold is about 60% to about 145%, such as any of about 65% to about 140%, about 70% to about 135%, about 75% to about 130%, about 80% to about 125%, about 85% to about 120%, or about 90% to about 115%, of the pharmacokinetic parameter of the reference PK curve of the drug. In some embodiments, the desired composite PK profile is determined based on having a pharmacokinetic parameter, such as any one or more of AUC, Cₘₐₓ, and tₘₐₓ, within an acceptable threshold of a reference PK curve of the drug, wherein the acceptable threshold is at least about an 80%, such as at least about any of 85%, 90%, or 95%, confidence interval that is within about 60% to about 145%, such as any of about 65% to about 140%, about 70% to about 135%, about 75% to about 130%, about 80% to about 125%, about 85% to about 120%, or about 90% to about 115%, of the pharmacokinetic parameter of the reference PK curve of the drug. In some embodiments, the desired composite PK profile is determined based on having a pharmacokinetic parameter, such as any one or more of AUC, Cₘₐₓ, and tₘₐₓ, within an acceptable threshold of a reference PK curve of the drug, wherein the acceptable threshold is at about a 90% confidence interval that is within about 80% to about 125% of the pharmacokinetic parameter of the reference PK curve of the drug.

In some embodiments, the desired composite PK profile is bioequivalent with a reference oral drug dosage form or a dosing regimen thereof, e.g., administration of the reference oral drug dosage form on a twice-a-day schedule. In some embodiments, the desired composite PK profile is bioequivalent with a reference oral drug dosage form, wherein the oral drug dosage form and the reference oral drug dosage form are administered at the same molar dose of a drug under the same conditions. In some embodiments, the desired composite PK profile is bioequivalent with a reference oral drug dosage form regimen, wherein the oral drug dosage form and the reference oral drug dosage form regimen are administered at the same molar dose of a drug under the same conditions. In some embodiments, the desired composite PK profile is a pharmaceutical alternative to a reference oral drug dosage form or a dosing regimen thereof, e.g., twice a day administration of the reference oral drug dosage form. In some embodiments, the desired composite PK profile is not significantly different in the rate and extent to which the active ingredient or active moiety in the oral drug dosage form becomes available at the site of drug action when compared to a reference, such as a reference oral drug dosage form or a dosing regimen thereof, e.g., twice a day administration of the reference oral drug dosage form, administered at the same molar dose under similar conditions in an appropriately designed study.

In some embodiments, the oral drug dosage form described herein having a desired PK profile is a bioequivalent of a reference oral drug dosage form or a dosing regimen thereof, wherein the 90% confidence interval for the ratio of the oral drug dosage form and the reference oral drug dosage form or the dosing regimen thereof falls within an about 80% to about 125% acceptance range for AUC and Cₘₐₓ. In some embodiments, the oral drug dosage form described herein having a desired PK profile is a bioequivalent of a reference oral drug dosage form or a dosing regimen thereof, wherein the 90% confidence interval for the ratio of the oral drug dosage form and the reference oral drug dosage form or the dosing regimen thereof falls within an about 80% to about 125% acceptance range for AUC, Cₘₐₓ, and tₘₐₓ.

In some embodiments, the desired PK profiles of the oral drug dosage forms described herein comprise an improved PK parameter, as compared to a reference, such as a reference oral drug dosage form or a dosing regimen thereof. In some embodiments, the improved PK parameter is an earlier Tₘₐₓ and/or a longer plateau period.

In some embodiments, the desired composite PK profile is a desired composite PK profile for a period of time. In some embodiment, the desired composite PK profile is for at least about 4 hours, such as at least about any of 6 hours, 8 hours, 10 hours, 12 hours, 14 hours, 16 hours, 18 hours, 20 hours, 22 hours, or 24 hours. In some embodiments, the desired composite PK profile is for at least about 4 hours, such as at least about any of 6 hours, 8 hours, 10 hours, 12 hours, 14 hours, 16 hours, 18 hours, 20 hours, 22 hours, or 24 hours, following administration of the drug to the individual.

In some embodiments, the reference, such as a reference PK curve of a drug, is a theoretical reference PK curve. In some embodiments, the reference PK curve of a drug is a measured reference PK curve. In some embodiments, the reference PK curve of a drug is a composite PK curve, e.g., based on two or more PK curves. In some embodiments, the reference PK curve is based on a dosing regimen of the drug, e.g., administration of the drug at least two or more times a day. In some embodiments, the reference oral drug dosage form or a dosing regimen thereof is an oral drug dosage form or a dosing regimen thereof that has approval by a government regulatory agency, e.g., the United States Food and Drug Administration (FDA). Techniques for measuring a PK curve of a drug, such as from an oral drug dosage form described herein or a reference oral drug dosage form, are known in the art. See, e.g., Heller et al., Annu Rev Anal Chem, 11, 2018; and Ghandforoush-Sattari et al., J Amino Acids, Article ID 346237, Volume 2010.

In some embodiments, the desired composite PK profile is predetermined. In some embodiments, the desired composite PK profile is based on a theoretical PK profile or PK curve. In some embodiments, the desired composite PK profile is based on a reference oral drug dosage form or a dosing regimen thereof, e.g., twice a day administration of the reference oral drug dosage form. In some embodiments, the desired composite PK profile is based on the PK curve of a reference oral drug dosage form or a dosing regimen thereof, e.g., twice a day administration of the reference oral drug dosage form. In some embodiments, the desired composite PK profile is based on a composite PK curve of a reference oral drug dosage form dosing regimen, e.g., twice a day administration of the reference oral drug dosage form.

In some embodiments, the desired composite PK profile is specific to an individual. In some embodiments, the individual is a human. In some embodiments, the individual is selected from the group consisting of a dog, a rodent, a mouse, a rat, a ferret, a pig, a guinea pig, a rabbit, and a non-human primate. In some embodiments, the desired composite PK profile is designed for a human.

In some embodiments, the dosage unit has a desired composite PK profile. In some embodiments, the PK curve of each of a plurality of dosage units results in an oral drug dosage form having a desired composite PK profile. In some embodiments, the PK curve of each of a plurality of dosage units, wherein each dosage unit is the same, results in an oral drug dosage form having a desired composite PK profile.

### Initial Design of the Dosage Units Described Herein

In some aspects, provided herein are methods of designing and/or producing an initial oral drug dosage form or a dosage unit having a release profile that can be adjusted based on the methods described herein to have a desired composite PK profile in an individual. In some embodiments, the initial dosage unit is adjusted by determining the relative amounts of the drug in the MR1 and MR2 portions based on the MR1 PK curve and the MR2 PK curve such that the MR1 portion and the MR2 portion when combined together produce the desired composite PK profile. In some embodiments, the initial dosage unit is adjusted by determining the relative amounts of the drug in the IR layer and ER layer based on the IR PK curve and ER PK curve such that the IR layer and the ER layer when combined together produce the desired composite PK profile.

In some embodiments, the initial dosage unit has a desired drug release profile, such as an in vitro drug release profile. Methods for designing and manufacturing dosage units having a desired release profile are known in the art. See, e.g., Goole et al., Int J Pharm, 499, 2016; and U.S. Pat. No. 10,350,822, which are both incorporated by reference in their entirety. Methods for in vitro dissolution testing include a logarithmic curve method, probability unit method, exponential model method, Weibull method, and Gompertz method. Statistical analysis methods for determining dissolution similarity of two dissolution profiles, e.g., an experimentally determined dissolution profile and a desired drug release profile, comprise regression analysis, ANOVA, similarity factor method, varying factor method, Splitpolt method, and Chow's method. In some embodiments, the dissolution similarity is evaluated using the similarity factor. In some embodiments, the dissolution similarity is evaluated using Chow's method.

In some embodiments, the method for designing the initial dosage unit comprises a step that is based on dissolution testing, such as in vitro dissolution testing. In some embodiments, the method comprises selecting one or more parameters for the ER layer to obtain a desired release profile, such as an in vitro release profile, of the drug from the ER layer. In some embodiments, the method comprises selecting one or more parameters for the IR layer to obtain a desired release profile, such as an in vitro release profile, of the drug from the IR layer. In some embodiments, the one or more parameters are selected from the group consisting of: thickness, surface area, substrate erosion rate, drug mass fraction or drug concentration, and layer configuration.

In some embodiments, the methods of designing an initial oral drug dosage form described herein may be performed, in whole or in part, on a computer system. In some embodiments, the computer system comprises a user interface. In some embodiments, the method comprises inputting one or more parameters of the oral drug dosage form into the computer system. In some embodiments, the computer system is used to calculate the parameters of the oral drug dosage form to provide a desired drug release profile. In some embodiments, the computer system comprises 3D drawing software. In some embodiments, the computer system is used to create a 3D drawing of the initial oral drug dosage form based on the pre-determined parameters of the initial oral drug dosage form. In some embodiments, the computer system comprises slicing software. In some embodiments, the computer system is used to convert a three-dimensional drawing of the initial oral drug dosage form into 3D printing code, e.g., G code. In some embodiments, the computer system executes the three-dimensional printing code, thereby printing the initial oral drug dosage form.

### Precursor Drug Dosage Forms

In some aspects, the methods described herein comprise obtaining a PK curve of a precursor drug dosage form in an individual. In some embodiments, the methods described herein comprise designing and/or producing a precursor drug dosage form, such as an IR precursor drug dosage form comprising the IR layer, and an ER precursor drug dosage form comprising the ER layer.

“Precursor drug dosage form,” as used herein, refers to a dosage form that models a portion of an oral drug dosage form or a dosage unit. In some embodiments, wherein the oral drug dosage form or dosage unit comprises an IR portion and an ER portion, the IR precursor drug dosage form comprises the IR portion and the ER precursor drug dosage form comprises the ER portion. In some embodiments, wherein the oral drug dosage form or dosage unit comprises an IR portion, an ER portion, and a shell, the IR precursor drug dosage form comprises the IR portion and the shell, and the ER precursor drug dosage form comprises the ER portion and the shell. In some embodiments, the individual components of a precursor drug dosage form, such as IR portion, ER portion, intermediate portion, and shell, are the same as are present in the oral drug dosage form or dosage unit. In some embodiments, the individual components of a precursor drug dosage form are situated in the same manner as are positioned in the oral drug dosage form or dosage unit.

In some embodiments, the method comprises obtaining, such as producing and/or 3D printing, a precursor drug dosage form, wherein the precursor drug dosage form is based on a dosage unit or an oral drug dosage form described herein. In some embodiments, the precursor drug dosage form is designed to simulate and test the contribution of a component, e.g., an ER layer, an IR layer, or an intermediate layer, of the dosage unit or the oral drug dosage form to the pharmacokinetics of said dosage unit or oral drug dosage form. In some embodiments, the precursor drug dosage form comprises a component, such as a single layer, e.g., an ER layer, an IR layer, or an intermediate layer, of the dosage unit or the oral drug dosage form. In some embodiments, the precursor drug dosage form further comprises a shell. In some embodiments, the component of the precursor drug dosage form is positioned in the precursor drug dosage form as it would be positioned in the dosage unit or the oral drug dosage form. In some embodiments, the component is a single component of the dosage unit or the oral drug dosage form. In some embodiments, the component is more than one component of the dosage unit or the oral drug dosage form. In some embodiments, the component of the precursor drug dosage form is not present in the dosage unit or the oral drug dosage form, e.g., use of a component to simulate the design of the dosage unit or the oral drug dosage form. In some embodiments, the component of the precursor drug dosage form is present to control an exposed surface (such as exposed to gastrointestinal fluid following administration to an individual) of another component, e.g., wherein the component is an intermediate layer. In some embodiments, a plurality of different precursor drug dosage forms can be obtained from a single dosage unit or a single oral drug dosage form.

In some embodiments, the precursor drug dosage form comprises an IR layer. In some embodiments, the precursor drug dosage form comprises an IR layer and a shell. In some embodiments, the precursor drug dosage form comprises an IR layer and an intermediate layer. In some embodiments, precursor drug dosage form comprises an IR layer, an intermediate layer, and a shell. In some embodiments, the precursor drug dosage form further comprises another component of the dosage unit or the oral drug dosage form, such as a second IR layer, an ER layer, an intermediate layer, or a shell.

In some embodiments, the precursor drug dosage form comprises an ER layer. In some embodiments, the precursor drug dosage form comprises an ER layer and a shell. In some embodiments, the precursor drug dosage form comprises an ER layer and an intermediate layer. In some embodiments, precursor drug dosage form comprises an ER layer, an intermediate layer, and a shell. In some embodiments, the precursor drug dosage form further comprises another component of the dosage unit or the oral drug dosage form, such as a second IR layer, an ER layer, an intermediate layer, or a shell.

In some embodiments, wherein the dosage unit comprises the IR layer stacked on top of the ER layer, a first precursor drug dosage form comprises the IR layer optionally stacked on top of a first intermediate layer, and a second precursor drug dosage form comprises the ER layer optionally stacked on top of a second intermediate layer. In some embodiments, the first intermediate layer is based on a property of the ER layer, e.g., dissolution rate. In some embodiments, the second intermediate layer is based on a property of the IR layer, e.g., dissolution rate.

In some embodiments, wherein the dosage unit comprises the ER layer stacked on top of the IR layer, the first precursor drug dosage form comprises the ER layer optionally stacked on top of a first intermediate layer, and the second precursor drug dosage form comprises the IR layer optionally stacked on top of a second intermediate layer. In some embodiments, the first intermediate layer is based on a property of the IR layer, e.g., dissolution rate. In some embodiments, the second intermediate layer is based on a property of the ER layer, e.g., dissolution rate.

In some embodiments, wherein the dosage unit comprises the IR layer stacked on top of the ER layer, wherein the IR layer and the ER layer are partially surrounded by a shell, and wherein the shell is in direct contact with both the IR layer and the ER layer and leaves only a top surface of the IR layer exposed, the first precursor drug dosage form comprises the IR layer partially surrounded by a first shell, wherein the first shell is in direct contact with the IR layer and leave the top surface of the IR layer exposed, and the second precursor drug dosage form comprises the ER layer partially surrounded by a second shell. In some embodiments, the second shell leaves the top surface of the ER layer exposed. In some embodiments, the second precursor drug dosage form further comprises an intermediate layer stacked on top of the ER layer, wherein the shell leaves a top surface of the intermediate layer exposed.

In some embodiments, wherein the dosage unit comprises the IR layer and the ER layer positioned side-by-side with each other, wherein the shell leaves the top surface of both the IR layer and the ER layer exposed, a first precursor drug dosage form comprises the IR layer, and optionally, a first intermediate layer side-by-side with the IR layer, and a first shell leaving the top surface of the IR layer exposed, a second precursor drug dosage form comprises the ER layer, and optionally, a second intermediate layer side-by-side with the ER layer, and a second shell leaving the top surface of the IR layer exposed.

In some embodiments, wherein the IR layer is stacked on top of the ER layer, and wherein the shell leaves the top surface of the IR layer and the bottom surface of the ER layer exposed, a first precursor drug dosage form comprises the IR layer, and optionally, an intermediate layer stacked on the bottom of the IR layer, wherein a first shell leaves the top surface of the IR layer and the bottom surface of the intermediate layer exposed, and a second precursor drug dosage form comprises the ER layer, and optionally, an intermediate layer stacked on top of the ER layer, wherein a second shell leaves the bottom surface of the ER layer and the top surface of the intermediate layer exposed

In some embodiments, the method comprises obtaining a PK curve of precursor drug dosage forms of a dosage unit in an individual. In some embodiments, the method comprises obtaining an ER PK curve of an ER precursor drug dosage form comprising the ER layer in the individual. In some embodiments, the method comprises: obtaining an IR PK curve of an IR precursor drug dosage form comprising the IR layer in the individual; and obtaining an ER PK curve of an ER precursor drug dosage form comprising the ER layer in the individual.

In some embodiments of the methods described herein, the PK curve of multiple ER precursor drug dosage forms are obtained and one or more ER precursor drug dosage forms are selected to be used for the oral drug dosage form or the dosage unit.

### Obtaining PK Curves

In some aspects, the methods described herein comprise obtaining, such as determining or measuring, a PK curve of a drug in an individual. In some embodiments, the method comprises obtaining an IR PK curve of an IR precursor drug dosage form comprising the IR laver in an individual. In some embodiments, the method comprises obtaining an ER PK curve of an ER precursor drug dosage form comprising the ER layer in the individual. In some embodiments, the method comprises obtaining an IR PK curve of an IR precursor drug dosage form comprising the IR layer in an individual, and obtaining an ER PK curve of an ER precursor drug dosage form comprising the ER layer in the individual. In some embodiments, the method comprises obtaining a PK curve of a dosage unit or an oral drug dosage form described herein. In some embodiments, the method comprises obtaining a PK curve of a reference oral drug dosage form or a dosing regimen thereof.

Techniques for obtaining a PK curve of a drug, such as from a dosing unit or an oral drug dosage form described herein, or a reference oral drug dosage form or a dosing regimen thereof, are known in the art. Se. e.g., Heller el al., Annu Rev Anal Chem, 11, 2018; and Ghandforoush-Sattari et al., J Amino Acids, Article ID 346237, Volume 2010. In some embodiments, the PK curve of the drug in the individual is measured in a blood, plasma, or serum sample from the individual. In some embodiments, the PK curve of the drug in the individual is measured using a mass spectrometry technique, such as LC-MS/MS.

In some embodiments, the PK curve of a drug is obtained for a period of at least about 3 half-lives of the drug, such as at least about any of 4 half-lives of the drug, 5 half-lives of the drug, 6 half-lives of the drug, 7 half-lives of the drug, 8 half-lives of the drug, 9 half-lives of the drug, or 10 half-lives of the drug, following administration of the drug to the individual. In some embodiments, the PK curve of a drug is obtained for a period of at least about 6 hours, such as at least about 8 hours, 10 hours, 12 hours, 14 hours, 16 hours, 18 hours, 20 hours, 22 hours, 24 hours, 36 hours, or 48 hours, following administration of the drug to the individual. In some embodiments, at least one or more of Cₘₐₓ, tₘₐₓ, and AUC may be obtained from the PK curve. In some embodiments, the AUC is limited by a time factor following administration of the drug to the individual, e.g., AUC_{0-6 hours} (AUC from 0-6 hours following administration). In some embodiments, pharmacokinetic parameters from a PK curve are assessed using a non-compartmental model.

In some embodiments, the PK curve of the drug is measured in an individual that is different than the individual for which the desired composite PK profile is designed for, e.g., the PK curve is measured in a dog and the desired composite PK profile is designed for a human.

In some embodiments, more than one PK curve is obtained. For example, in some embodiments, at least 2, such as at least any of 3, 4, 5, 10, or 15, PK curves are obtained for an IR precursor drug dosage form and/or an ER precursor drug dosage form.

In some embodiments, PK curves for a plurality of ER precursor drug dosage forms are obtained, wherein at least two of the plurality of ER precursor drug dosage forms have a different configuration, such as a different parameter is selected from layer surface area, thickness, and erosion rate.

In some embodiments, two or more PK curves are combined to obtain a composite PK curve. In some embodiments, the two or more PK curves comprise PK curves of at least two different dosage forms, e.g., a PK curve of an IR precursor drug dosage form and a PK curve of an ER precursor drug dosage form. In some embodiments, the two or more PK curves are obtained from the same individual. In some embodiments, the two or more PK curves are obtained from at least two different individuals.

### Determining Relative Amounts of the Drug in Portions of the Oral Drug Dosage Form

The methods described herein comprise determining the relative amounts of the drug in the MR1 portion and the MR2 portion based on PK curves. In some embodiments, the method comprises determining the relative amounts of the drug in the IR layer and the ER layer based on the IR PK curve of an IR precursor drug dosage form comprising the IR layer in the individual and the ER PK curve of an ER precursor drug dosage form comprising the ER layer in the individual, such that the IR layer and ER layer when combined together to form the dosage unit or the oral drug dosage form produce the desired composite PK profile. In some embodiments, the method comprises determining the relative amounts of the drug in the IR layer and the ER layer based on the ER PK curve of an ER precursor drug dosage form comprising the ER layer in the individual.

In some embodiments, determining the relative amounts of the drug in an IR layer and an ER layer of a dosage unit is based on theoretical PK simulations of exemplary oral drug dosage forms having different IR:ER drug ratios, wherein theoretical simulations are based on a PK curve or an JR precursor drug dosage form and a PK curve of an ER precursor drug dosage form.

In some embodiments, the amounts of the drug in the IR layer and the ER layer are determined based on drug in vivo dynamic information, such as an in vivol in vitro correlation (IVIVC). In some embodiments, the IVIVC is based on the in vitro release and m vivo performance of a characterized drug, obtained using deconvolution based on PK data, such as PK data obtained from one or more PK curves. In some embodiments, the amounts of the drug in the IR layer and the ER layer are determined based on a point-to-point relationship of in vitro dissolution rate and in vivo dissolution rate (input rate) of the drug. In some embodiments, the each point of the point-to-point relationship is based on a time after administration time point. In some embodiments, the point-to-point relationship is calculated to determine the in vitro release end point of the extended release portion of a dosage unit or an oral drug dosage form, such as an ER layer, corresponding to the in vivo release end point, thereby allowing for an immediate release portion of the dosage unit or the oral drug dosage form, such as an IR layer, to be added to assess composite PK information.

In some embodiments, the deconvolution method is suitable for IVIVC calculation of PK curves in animals, such as dogs and rodents, and humans. In some embodiments, the PK curve of a drug obtained from a human is more complicated than a PK curve of the drug obtained from an animal, such as a dog or a rodent. In some embodiments, the IVIVC curve of in vitro and in vivo dissolution can be obtained based on a physiologically based pharmacokinetic (PBPK) model.

In some embodiments, the methods described herein comprise adjusting a parameter of a layer of the dosage unit or the oral drug dosage form, such as the IR layer or the ER layer. In some embodiments, parameter is selected from layer surface area, thickness, erosion rate. In some embodiments, the adjusting of a parameter of a layer is performed to adjust the drug release profile of said layer.

### Exemplary Methods of Designing an Oral Drug Dosage Form Described Herein

In some embodiments, provided herein is a method of designing an oral drug dosage form having a fixed amount of a drug and a desired composite pharmacokinetic (PK) profile in an individual, wherein the oral drug dosage form comprises a dosage unit comprising: an immediate-release (IR) portion comprising the drug, the IR portion having an immediate-release profile; and an extended release (ER) portion comprising the drug, the ER portion having an extended-release profile, the method comprising: determining the relative amounts of the drug in the IR portion and the ER portion based on an IR PK curve of an IR precursor drug dosage form comprising the IR portion in the individual, and an ER PK curve of an ER precursor drug dosage form comprising the ER portion in the individual such that the IR portion and the ER portion when combined together produce the oral drug dosage form having the desired composite PK profile in the individual. In some embodiments, the method further comprises obtaining the ER PK curve of the ER precursor drug dosage form comprising the ER portion in the individual. In some embodiments, the method further comprises obtaining the IR PK curve of the IR precursor drug dosage form comprising the IR portion in the individual.

In some embodiments, provided herein is a method of designing an oral drug dosage form having a fixed amount of a drug and a desired composite pharmacokinetic (PK) profile in an individual, wherein the oral drug dosage form comprises a dosage unit comprising: an immediate-release (IR) layer comprising the drug, the IR layer having an immediate-release profile; and an extended release (ER) layer comprising the drug, the ER layer having an extended-release profile, wherein the IR layer and the ER layer are stacked on top of each other, wherein the IR layer and ER layer are partially surrounded by a shell, wherein the shell has a slower dissolution rate than the ER layer, wherein the IR layer has a top surface and a bottom surface, wherein the ER layer has a top surface and a bottom surface, and wherein the shell is in direct contact with both IR layer and the ER layer and leaves only the top surface of the IR layer exposed, the method comprising: (a) obtaining an IR PK curve of an IR precursor drug dosage form comprising the IR layer in the individual; (b) obtaining an ER PK curve of an ER precursor drug dosage form comprising the ER layer in the individual; and (c) determining the relative amounts of the drug in the IR layer and ER layer based on the IR PK curve and ER PK curve such that the IR layer and the ER layer when combined together produce the desired composite PK profile, thereby designing the oral drug dosage form having the desired composite PK profile in the individual. In some embodiments, the individual is a human. In some embodiments, the drug has linear pharmacokinetics. In some embodiments, the drug has linear pharmacokinetics for the concentration of the drug administered to the individual. In some embodiments, the dosage unit further comprises a second IR layer, wherein the second IR layer has a top surface and a bottom surface, wherein the ER layer is stacked on top of the second IR layer, and wherein the shell leaves only the top surface of the IR layer exposed. In some embodiments, the dosage unit further comprises an intermediate layer positioned between the IR layer and the ER layer and/or the second IR layer and the ER layer.

In some embodiments, provided herein is a method of designing an oral drug dosage form having a fixed amount of a drug and a desired composite pharmacokinetic (PK) profile in an individual, wherein the oral drug dosage form comprises a dosage unit comprising: an immediate-release (IR) layer comprising the drug, the IR layer having an immediate-release profile; and an extended release (ER) layer comprising the drug, the ER layer having an extended-release profile, wherein the IR layer and the ER layer are stacked on top of each other, wherein the IR layer and ER layer are partially surrounded by a shell, wherein the shell has a slower dissolution rate than the ER layer, wherein the IR layer has a top surface and a bottom surface, wherein the ER layer has a top surface and a bottom surface, and wherein the shell is in direct contact with both 1R layer and the ER layer and leaves only the top surface of the ER layer exposed, the method comprising: (a) obtaining an IR PK curve of an IR precursor drug dosage form comprising the IR layer in the individual; (b) obtaining an ER PK curve of an ER precursor drug dosage form comprising the ER layer in the individual; and (c) determining the relative amounts of the drug in the IR layer and ER layer based on the IR PK curve and ER PK curve such that the IR layer and the ER layer when combined together produce the desired composite PK profile, thereby designing the oral drug dosage form having the desired composite PK profile in the individual. In some embodiments, the individual is a human. In some embodiments, the drug has linear pharmacokinetics. In some embodiments, the drug has linear pharmacokinetics for the concentration of the drug administered to the individual.

In some embodiments, provided herein is a method of designing an oral drug dosage form having a fixed amount of a drug and a desired composite pharmacokinetic (PK) profile in an individual, wherein the oral drug dosage form comprises a dosage unit comprising: an immediate-release (IR) layer comprising the drug, the IR layer having an immediate-release profile; and an extended release (ER) layer comprising the drug, the ER layer having an extended-release profile, wherein the IR layer and the ER layer are stacked on top of each other, wherein the IR layer and ER layer are partially surrounded by a shell, wherein the shell has a slower dissolution rate than the ER layer, wherein the IR layer has a top surface and a bottom surface, wherein the ER layer has a top surface and a bottom surface, and wherein the shell is in direct contact with both IR layer and the ER layer and leaves the top surface of the IR layer and the bottom surface of the ER layer exposed, the method comprising: (a) obtaining an IR PK curve of an IR precursor drug dosage form comprising the JR layer in the individual; (b) obtaining an ER PK curve of an ER precursor drug dosage form comprising the ER layer in the individual; and (c) determining the relative amounts of the drug in the IR layer and ER layer based on the IR PK curve and ER PK curve such that the IR layer and the ER layer when combined together produce the desired composite PK profile, thereby designing the oral drug dosage form having the desired composite PK profile in the individual. In some embodiments, the individual is a human. In some embodiments, the drug has linear pharmacokinetics. In some embodiments, the drug has linear pharmacokinetics for the concentration of the drug administered to the individual. In some embodiments, the dosage unit further comprises an intermediate layer positioned between the IR layer and the ER layer.

In some embodiments, provided herein is a method of designing an oral drug dosage form having a fixed amount of a drug and a desired composite pharmacokinetic (PK) profile in an individual, wherein the oral drug dosage form comprises a dosage unit comprising: an immediate-release (IR) layer comprising the drug, the IR layer having an immediate-release profile; and an extended release (ER) layer comprising the drug, the ER layer having an extended-release profile, the IR layer and the ER layer are positioned side-by-side with each other, wherein the IR layer and ER layer are partially surrounded by a shell, wherein the shell has a slower dissolution rate than the ER layer, wherein the IR layer has a top surface and a bottom surface, wherein the ER layer has a top surface and a bottom surface, and wherein the shell is in direct contact with both IR layer and the ER layer and leaves the top surface of both the IR layer and the ER layer exposed, the method comprising: (a) obtaining an IR PK curve of an IR precursor drug dosage form comprising the JR layer in the individual; (b) obtaining an ER PK curve of an ER precursor drug dosage form comprising the ER layer in the individual; and (c) determining the relative amounts of the drug in the IR layer and ER layer based on the IR PK curve and ER PK curve such that the IR layer and the ER layer when combined together produce the desired composite PK profile, thereby designing the oral drug dosage form having the desired composite PK profile in the individual. In some embodiments, the individual is a human. In some embodiments, the drug has linear pharmacokinetics. In some embodiments, the drug has linear pharmacokinetics for the concentration of the drug administered to the individual. In some embodiments, the dosage unit further comprises an intermediate layer positioned between the IR layer and the ER layer.

In some embodiments, provided herein is a method of designing an oral drug dosage form having a fixed amount of a drug and a desired composite pharmacokinetic (PK) profile in an individual, wherein the oral drug dosage form comprises a dosage unit comprising: an immediate-release (IR) layer comprising the drug, the IR layer having an immediate-release profile; and an extended-release (ER) layer comprising the drug, the ER layer having an extended-release profile, wherein the IR layer and the ER layer are stacked on top of each other, and wherein, at the fixed amount of the drug, the drug has linear pharmacokinetics, the method comprising: (a) obtaining an IR PK curve of an IR precursor drug dosage form comprising the IR portion in the individual; (b) obtaining an ER PK curve of an ER precursor drug dosage form comprising the ER portion in the individual; and (c) determining the relative amounts of the drug in the IR layer and the ER layer based on the IR PK curve and ER PK curve such that the IR layer and the ER layer when combined together produce the oral drug dosage form having the desired composite PK profile in the individual.

In some embodiments, provided herein is a method of designing an oral drug dosage form having a fixed amount of a drug and a desired composite pharmacokinetic (PK) profile in a human, wherein the oral drug dosage form comprises a dosage unit comprising: an immediate-release (JR) layer comprising the drug, the IR layer having an immediate-release profile; and an extended-release (ER) layer comprising the drug, the ER layer having an extended-release profile, wherein the IR layer and the ER layer are stacked on top of each other, and wherein, at the fixed amount of the drug, the drug has linear pharmacokinetics, the method comprising: (a) obtaining an IR PK curve of an IR precursor drug dosage form comprising the IR portion in the human; (b) obtaining an ER PK curve of an ER precursor drug dosage form comprising the ER portion in the human; and (c) determining the relative amounts of the drug in the IR layer and the ER layer based on the IR PK curve and ER PK curve such that the IR layer and the ER layer when combined together produce the oral drug dosage form having the desired composite PK profile in the human.

In some embodiments, provided herein is a method of designing an oral drug dosage form having a fixed amount of a drug and a desired composite pharmacokinetic (PK) profile in an individual, wherein the oral drug dosage form comprises a dosage unit comprising: an immediate-release (IR) layer comprising the drug, the IR layer having an immediate-release profile; and an extended-release (ER) layer comprising the drug, the ER layer having an extended-release profile, wherein the IR layer and the ER layer are stacked on top of each other, and wherein, at the fixed amount of the drug, the drug has linear pharmacokinetics, the method comprising: (a) obtaining an IR PK curve of an IR precursor drug dosage form comprising the IR portion in the individual; (b) obtaining an ER PK curve of an ER precursor drug dosage form comprising the ER portion in the individual; and (c) determining the relative amounts of the drug in the IR layer and the ER layer based on the JR PK curve and ER PK curve such that the IR layer and the ER layer when combined together produce the oral drug dosage form having the desired composite PK profile in the individual, wherein the time ranges of the desired composite PK profile, the IR PK curve, and the ER PK curve are each between 0 hours and about 24 hours.

In some embodiments, provided herein is a method of designing an oral drug dosage form having a fixed amount of a drug and a desired composite pharmacokinetic (PK) profile in an individual, wherein the oral drug dosage form comprises a dosage unit comprising: an immediate-release (IR) layer comprising the drug, the IR layer having an immediate-release profile; and an extended-release (ER) layer comprising the drug, the ER layer having an extended-release profile, wherein the IR layer and the ER layer are stacked on top of each other, and wherein the desired composite PK profile is determined based on having an area under the curve (AUC), a Cₘₐₓ, and a tₘₐₓ within an acceptable threshold of a reference PK curve of the drug, the method comprising: (a) obtaining an IR PK curve of an IR precursor drug dosage form comprising the IR portion in the individual; (b) obtaining an ER PK curve of an ER precursor drug dosage form comprising the ER portion in the individual; and (c) determining the relative amounts of the drug in the IR layer and the ER layer based on the IR PK curve and ER PK curve such that the IR layer and the ER layer when combined together produce the oral drug dosage form having the desired composite PK profile in the individual.

In some embodiments, provided herein is a method of determining the relative amounts of a drug in an immediate-release (IR) portion and an extended-release (ER) portion of an oral drug dosage form having a fixed amount of a drug and a desired composite pharmacokinetic (PK) profile in an individual, wherein the oral drug dosage form comprises a dosage unit comprising: the IR portion comprising the drug, the IR portion having an immediate-release profile, and the ER portion comprising the drug, the ER portion having an extended-release profile, the method comprising, determining the relative amounts of the drug in the IR portion and the ER portion based on an IR PK curve of an IR precursor drug dosage form comprising the IR portion in the individual, and an ER PK curve of an ER precursor drug dosage form comprising the ER portion in the individual such that the IR portion and the ER portion when combined together produce the oral drug dosage form having the desired composite PK profile in the individual.

In some embodiments, provided herein is a method of producing an oral drug dosage form having a fixed amount of a drug and a desired composite pharmacokinetic (PK) profile in an individual, wherein the oral drug dosage form comprises a dosage unit comprising: the IR portion comprising the drug, the IR portion having an immediate-release profile; and the ER portion comprising the drug, the ER portion having an extended-release profile, the method comprising: determining the relative amounts of the drug in the IR portion and the ER portion based on an IR PK curve of an IR precursor drug dosage form comprising the IR portion in the individual, and an ER PK curve of an ER precursor drug dosage form comprising the ER portion in the individual such that the IR portion and the ER portion when combined together produce the oral drug dosage form having the desired composite PK profile in the individual.

In some embodiments, the methods described herein further comprise determining the IR PK curve and the ER PK curve and adjusting the relative amounts of the drug in the IR portion and the ER portion. In some embodiments, the methods described herein further comprise determining a composite PK curve of the oral drug dosage form. In some embodiments, the methods described herein further comprise adjusting the relative amounts of the drug in the IR layer and the ER layer based on a comparison of the composite PK curve and the desired composite PK profile.

In some embodiments, the methods described herein further comprise producing the oral drug dosage form. In some embodiments, the oral drug dosage form is produced by three-dimensional printing. In some embodiments, the three-dimensional printing is carried out by fused deposition modeling (FDM).

### Methods of Three-Dimensional Printing

In some aspects, the present disclosure provides methods of printing, such as three-dimensional (3D) printing, an oral drug dosage form having a desired composite pharmacokinetic (PK) profile, or a component, such as a dosage unit, or a precursor thereof.

In some embodiments, the method comprises 3D printing an oral drug dosage form described herein. In some embodiments, the method comprises 3D printing a component of an oral drug dosage form described herein, such as dosage unit or a component thereof, e.g., an IR layer, an ER layer, an intermediate layer, or a shell. In some embodiments, the method comprises 3D printing a precursor drug dosage form, such as an IR precursor drug dosage form or an ER precursor drug dosage form.

As used herein, “printing,” “three-dimensional printing,” “3D printing,” “additive manufacturing,” or equivalents thereof, refers to a process that produces three-dimensional objects, such as drug dosage forms, layer-by-layer using digital designs. The basic process of three-dimensional printing has been described in U.S. Pat. Nos. 5,204,055; 5,260,009; 5,340,656; 5,387,380; 5,503,785; and 5,633.021. Additional U.S. patents and patent applications that related to three-dimensional printing include: U.S. Pat. Nos. 5,490,962; 5,518,690; 5,869,170; 6,530,958; 6,280,771; 6,514,518; 6,471,992; 8,828,411; U.S. Publication Nos. 2002/0015728; 2002/0106412; 2003/0143268; 2003/0198677; 2004/0005360. The contents of the above U.S. patents and patent applications are hereby incorporated by reference in their entirety.

In some embodiments, an additive manufacturing technique is used to produce the drug dosage forms described herein. In some embodiments, a layer-by-layer technique is used to produce the drug dosage forms described herein.

Different 3D printing methods have been developed for drug dosage form manufacturing in terms of raw materials, equipment, and solidification. These 3D printing methods include binder deposition (see Gibson et al., Additive Manufacturing Technologies: 3D Printing Rapid Prototyping, and Direct Digital Manufacturing, 2 ed. Springer, New York, 2015; Katstra et al., Oral dosage forms fabricated by three dimensional printing, J Control Release, 66, 2000; Katstra et al., Fabrication of complex oral delivery forms by three dimensional printing, Dissertation in Materials Science and Engineering, Massachusetts Institute of Technology, 2001; Lipson et al., Fabricated: The New World of 3D printing, John Wiley & Sons, Inc., 2013: Jonathan, Karim 3D printing in pharmaceutics: a new tool for designing customized drug delivery systems, Int J Pharm, 499, 2016), material jetting (see Jonathan, Karim, 3D printing in pharmaceutics: a new tool for designing customized drug delivery systems, Int J Pharm, 499, 2016), extrusion (see Gibson et al., Additive Manufacturing Technologies: 3D Printing, Rapid Prototyping, and Direct Digital Manufacturing. 2 ed. Springer, New York, 2015), and photopolymerization (see Melchels et al., A review on stereolithography and its application in biomedical engineering. Biomaterials, 31, 2010).

In some embodiments, the oral drug dosage forms described herein are 3D printed using an extrusion method. In some embodiments, the method of 3D printing comprises using a double screw extrusion method. In an extrusion process, material is extruded from robotically-actuated printing heads through printing nozzles. Unlike binder deposition, which requires a powder bed, extrusion methods can print on any substrate. A variety of materials can be extruded for three-dimensional printing, including thermoplastic materials disclosed herein, pastes and colloidal suspensions, silicones, and other semisolids. One common type of extrusion printing is fused deposition modeling, which uses solid polymeric filaments for printing. In fused deposition modeling, a gear system drives the filament into a heated nozzle assembly for extrusion (see Gibson et al., Additive Manufacturing Technologies: 3D Printing, Rapid Prototyping, and Direct Digital Manufacturing, 2 ed. Springer, New York, 2015).

In some embodiments, the 3D printing methods described herein comprise a continuous feed method.

In some embodiments, the 3D printing methods described herein comprise a batch feed method.

In some embodiments, the 3D printing is carried out by fused deposition modeling (FDM). In some embodiments, the 3D printing is carried out by non-filament FDM. In some embodiments, the 3D printing is carried out by melt extrusion deposition (MED). In some embodiments, the 3D printing is carried out by inkjet printing. In some embodiments, the 3D printing is carried out by selective laser sintering (SLS). In some embodiments, the 3D printing is carried out by stereolithography (SLA or SL). In some embodiments, the 3D printing is carried out by PolyJet, Multi-Jet Printing System (MJP), Perfactory, Solid Object Ultraviolet-Laser Printer, Bioplotter, 3D Bioprinting, Rapid Freeze Prototyping, Benchtop System, Selective Deposition Lamination (SDL), Laminated Objet Manufacturing (LOM), Ultrasonic Consolidation, ColorJet Printing (CJP), EOSINT Systems, Laser Engineered Net Shaping (LENS) and Aerosol Jet System, Electron Beam Melting (EBM), Laser CUSING®, Selective Laser Melting (SLM), Phenix PXTM Series, Microsintering, Digital Part Materialization (DPM), or VX System.

In some embodiments, the three-dimensional printing is carried out by hot melt extrusion coupled with a 3D printing technique, such as FDM. In some embodiments, the three-dimensional printing is carried out by melt extrusion deposition (MED).

In some embodiments, the methods for producing the oral drug dosage forms described herein comprise a 3D printing technique, such as 3D printing in combination with another method, e.g., a combination of injection molding and 3D printing. In some embodiments, the shell is produced using injection molding and one or more modulated-release portions is produced using a 3D printing technique.

The method instructions for 3D printing a drug dosage form disclosed herein may be generated a variety of ways, including direct coding, derivation from a solid CAD model, or other means specific to the 3D printing machine's computer interface and application software. These instructions may include information on the number and spatial placement of droplets, and on general 3D print parameters such as the drop spacing in each linear dimension (X, Y, Z), and volume or mass of fluid per droplet. For a given set of materials, these parameters may be adjusted in order to refine the quality of structure created. The overall resolution of the structure created is a function of the powder particle size, the fluid droplet size, the print parameters, and the material properties.

Because 3D printing may handle a range of pharmaceutical materials and control both composition and architecture locally, 3D printing is well suited to the fabrication of oral drug dosage forms with complex geometry and composition in accordance with the present invention.

In some embodiments, wherein the oral drug dosage form comprises more than one dosage unit, each dosage unit is printed separately and later assembled to form the oral drug dosage form. In some embodiments, wherein the oral drug dosage form comprises more than one dosage unit, the more than one dosage unit are printed as the formed oral drug dosage form.

The oral drug dosage forms and components thereof described in the present application can be printed on a commercial scale. For example, in some embodiments, the methods disclosed herein may be used to 3D print 10,000 to 100,000 units of an oral drug dosage form per hour. In some embodiments, the methods disclosed herein may be used to 3D print 10,000 to 100,000 oral drug dosage forms per hour. In some embodiments, the methods disclosed herein may be used to 3D print 10,000 to 100,000 units of a dosage unit per hour. In some embodiments, the methods disclosed herein may be used to 3D print 10,000 to 100,000 dosage units per hour.

Manufacturing the drug dosage forms using 3D printing methods also facilitates personalized medicine. Personalized medicine refers to stratification of patient populations based on biomarkers to aid therapeutic decisions and personalized dosage form design. Modifying digital designs is easier than modifying physical equipment. Also, automated, small-scale three-dimensional printing may have negligible operating cost. Hence, 3D printing can make multiple small, individualized batches economically feasible and enable personalized dosage forms designed to improve adherence.

Personalized drug dosage forms allow for tailoring the amount of drug delivered based on a patient's mass and metabolism. 3D printed dosage forms could ensure accurate dosing in growing children and permit personalized dosing of highly potent drugs. Personalized dosage forms can also combine all of patients' medications into a single daily dose, thus improve patients' adherence to medication and treatment compliance.

In some embodiments, the method comprises: dispensing an IR material to produce the IR layer comprising the drug. In some embodiments, multiple layers of the IR material are dispensed to produce the IR layer. In some embodiments, the IR layer has a pre-determined surface area, thickness, and drug mass fraction. In some embodiments, the method comprises: dispensing an ER material to produce the ER layer comprising the drug. In some embodiments, multiple layers of the ER material are dispensed to produce the ER layer. In some embodiments, the ER layer has a pre-determined surface area, thickness, and drug mass fraction. In some embodiments, the method comprises: dispensing an intermediate material to produce the intermediate layer comprising the drug. In some embodiments, multiple layers of the intermediate material are dispensed to produce the intermediate layer. In some embodiments, the intermediate layer has a pre-determined surface area and thickness. In some embodiments, the method comprises: dispensing a shell material to produce the shell comprising the drug. In some embodiments, multiple layers of the shell material are dispensed to produce the shell. In some embodiments, the shell has a pre-determined surface area and thickness.

In some embodiments, the method comprises: dispensing a shell material to produce the shell or a portion thereof; dispensing an ER material comprising the drug on top of the shell or portion thereof to produce the ER layer; and dispensing an IR material comprising the drug on top of the ER layer to produce the IR layer, thereby printing the oral drug dosage form or the dosage unit. In some embodiments, the method further comprises dispensing an intermediate material to produce the intermediate layer, wherein the intermediate layer is positioned as described herein.

In some embodiments, the method comprises: dispensing a shell material to produce the shell or a portion thereof; dispensing an IR material comprising the drug on top of the shell or portion thereof to produce the IR layer; and dispensing an ER material comprising the drug on top of the IR layer to produce the ER layer, thereby printing the oral drug dosage form or the dosage unit. In some embodiments, the method further comprises dispensing the IR material comprising the drug on top of the ER layer to produce a second IR layer. In some embodiments, the method further comprises dispensing an intermediate material to produce the intermediate layer, wherein the intermediate layer is positioned as described herein.

In some embodiments, the method comprises: dispensing a shell material to produce the shell or a portion thereof; dispensing an IR material comprising the drug on top of the shell or portion thereof to produce the IR layer; and dispensing an ER material comprising the drug on top of the shell or portion thereof to produce the ER layer, thereby printing the oral drug dosage form or the dosage unit, wherein the IR layer and the ER layer are positioned side-by-side. In some embodiments, the method further comprises dispensing an intermediate material to produce the intermediate layer, wherein the intermediate layer is positioned as described herein.

In some embodiments, the materials used to print the oral drug dosage forms and dosage units, or components thereof, e.g., precursor drug dosage forms, are each dispensed by a different printing head. For example, in some embodiments, the IR material and the ER material, and optionally if present, the intermediate material and the shell material, are each dispensed by a different printing head.

The 3D printing methods described herein encompass printing the materials in any order that will allow for production of the oral drug dosage form and dosage units, or components thereof, e.g., precursor drug dosage forms, disclosed herein.

In some embodiments, the method for 3D printing comprises designing the oral drug dosage form or dosage unit, or component thereof, e.g., a precursor drug dosage form, in whole or in part, on a computer system. In some embodiments, the method comprises inputting parameters of the desired drug release profile and/or the oral drug dosage form and/or the dosage unit and/or a precursor drug dosage form into the computer system. In some embodiments, the method comprises providing one or more parameters to be printed, e.g., layer surface area, thickness, drug mass fraction, erosion rate. In some embodiments, the method comprises providing the desired drug release profile. In some embodiments, the methods comprise creating a virtual image of the item to be printed. In some embodiments, the method comprises creating a computer model that contains the pre-determined parameters. In some embodiments, the method comprises feeding the pre-determined parameters to a 3D printer and printing the item according to such pre-determined parameters. In some embodiments, the method comprises creating a 3D drawing of the item to be printed based on the pre-determined parameters, wherein the 3D drawing is created on a computer system. In some embodiments, the method comprises converting, such as slicing, a 3D drawing into 3D printing code, e.g., G code. In some embodiments, the method comprises using the computer system to execute 3D printing code, thereby printing according to the methods described herein.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXAMPLES

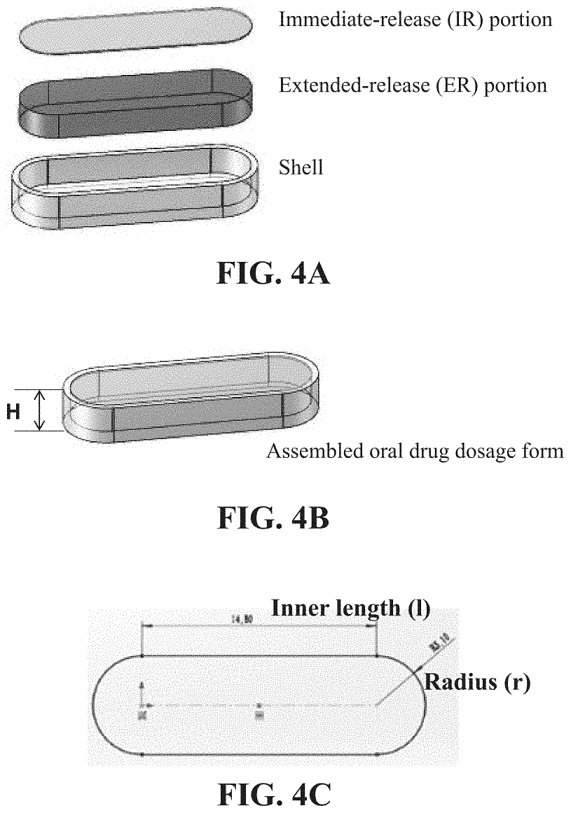

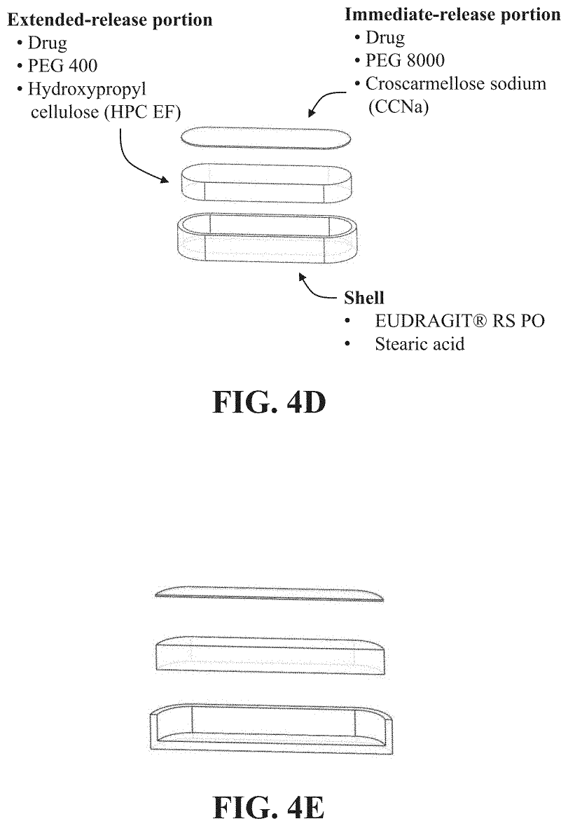

The examples below demonstrate the design of exemplary oral drug dosage forms having a fixed amount of a drug and a desired composite pharmacokinetic (PK) profile in an individual (e.g., FIGS. 4A-4E, FIG. 2E) using the novel 3D Printing Formulation by Design (3DPFbD®) approach described herein.

### Example 1

An initial oral drug dosage form comprising a fixed amount of drug, an immediate-release (IR) portion, and an extended-release (ER) portion was designed. See, e.g., FIGS. 4A and 4D (showing deconstructed views of the oral drug dosage form), and FIG. 4B (showing an assembled view of the oral drug dosage form; the ER portion and the IR portion are stacked and fit in the space formed by the shell).

To obtain an immediate-release (IR) pharmacokinetic (PK) curve to measure the drug plasma concentration attributable to the IR portion of the oral drug dosage form, a corresponding IR precursor drug dosage form comprising the designed IR portion and shell (excluding the ER portion of the oral drug dosage form) was printed by 3D printing. The materials and dimensions of the IR precursor drug dosage form are provided in Table 1 and Table 2. The dimensions provided are the outside boundaries. The IR portion of the IR precursor drug dosage form was manufactured to contain 50 mg of the drug. The PK curve of the IR precursor drug dosage form and the PK curve of an immediate-release reference drug dosage form (IR reference drug dosage form) having the same drug amount as the IR precursor drug dosage form were obtained according to the methods described herein.

TABLE 1
Composition of components of the IR precursor drug dosage form.

| | Weight/tablet | | |
|---|---|---|---|
| Module | (mg) | Material | % |
| Immediate-release | 83.3 | Drug | 60 |
| portion | | Polyethylene glycol (PEG) 8000 | 35 |
| | | Croscarmellose sodium (CCNa) | 5 |
| Shell | — | EUDRAGIT ® RS PO | 90 |
| | | Stearic acid | 10 |

TABLE 2
Dimensions of the components of the IR precursor drug dosage form.

| Module | Radius (mm) | Inner length (mm) | Thickness (mm) |
|---|---|---|---|
| Immediate-release | 3 | 13 | 0.6 |
| portion | | | |
| Shell | 3.6 | 13 | 1.4 |

To obtain an extended-release (ER) pharmacokinetics (PK) curve to measure the drug plasma concentration attributable to the ER portion of the oral drug dosage form, a corresponding ER precursor drug dosage form comprising the designed ER portion and shell (excluding the IR portion of the oral drug dosage form) was printed by 3D printing. The materials and dimensions of the ER precursor drug dosage form are provided in Table 3 and Table 4. The ER portion of the ER precursor drug dosage form was manufactured to contain 87.5 mg of the drug. The PK curve of the ER precursor drug dosage form and the PK curve of an extended-release reference drug dosage form (ER reference) having the same drug amount as the ER precursor drug dosage form were obtained according to the methods described herein.

TABLE 3
Composition of components of the ER precursor dosage form.

| | Weight/tablet | | |
|---|---|---|---|
| Module | (mg) | Material | % |
| Extended-release | 291.7 | Drug | 30 |
| portion | | Hydroxypropyl cellulose (HPC EF) | 50 |
| | | Polyethylene glycol (PEG) 400 | 20 |
| Shell | — | EUDRAGIT ® RS PO | 90 |
| | | Stearic acid | 10 |

TABLE 4
Dimensions of the components of the ER precursor dosage form.

| Module | Radius (mm) | Inner length (mm) | Thickness (mm) |
|---|---|---|---|
| Extended-release | 3 | 12.5 | 3 |
| portion | | | |
| Shell | 3.6 | 12.5 | 3.8 |

In vivo pharmacokinetic studies were performed in fasted male beagle dogs. After oral administration of the respective precursor drug dosage forms or reference drug dosage forms, blood samples were collected from the jugular vein at predetermined times and the plasma concentration of the drug was determined by LC-MS/MS analysis.

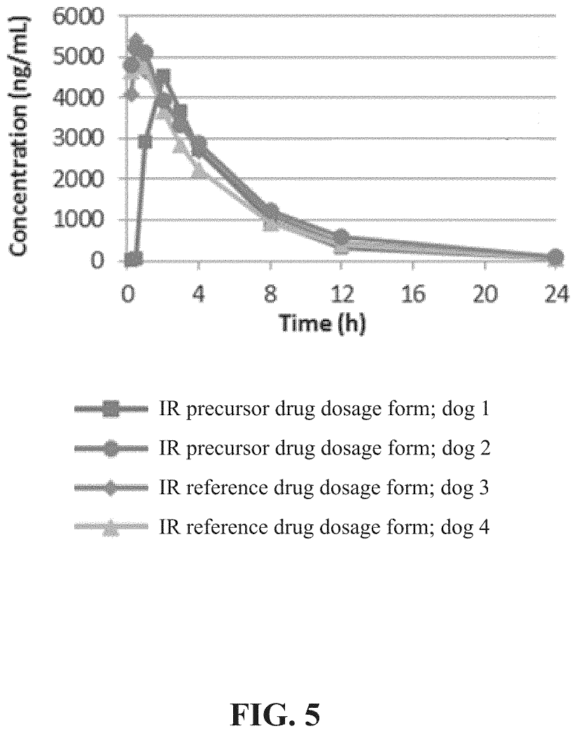

The 3D-printed IR precursor drug dosage form has a similar in vivo PK curve as the IR reference drug dosage form, thus demonstrating bioequivalence (FIG. 5).

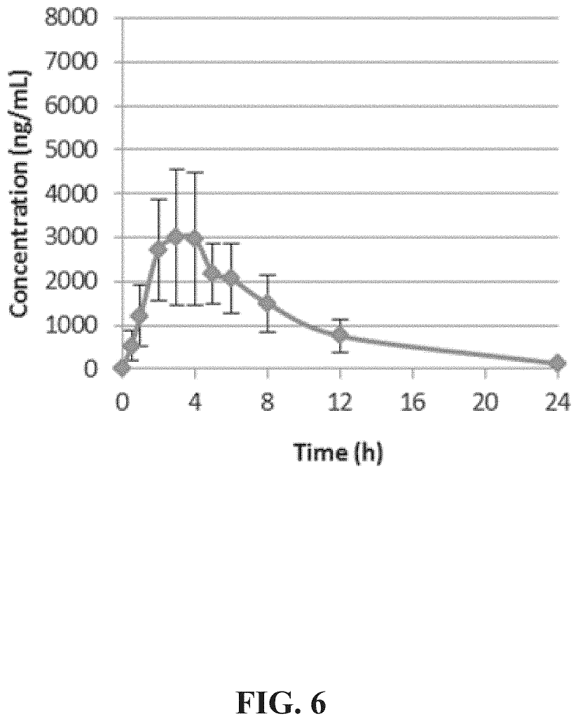

The 3D-printed ER precursor drug dosage form demonstrated an ER PK curve with a long and slowly descending plateau after reaching Cₘₐₓ (FIG. 6). The in vitro dissolution rate of the 3D-printed ER precursor drug dosage form was measured to be −16 hours (data not shown).

### Example 2

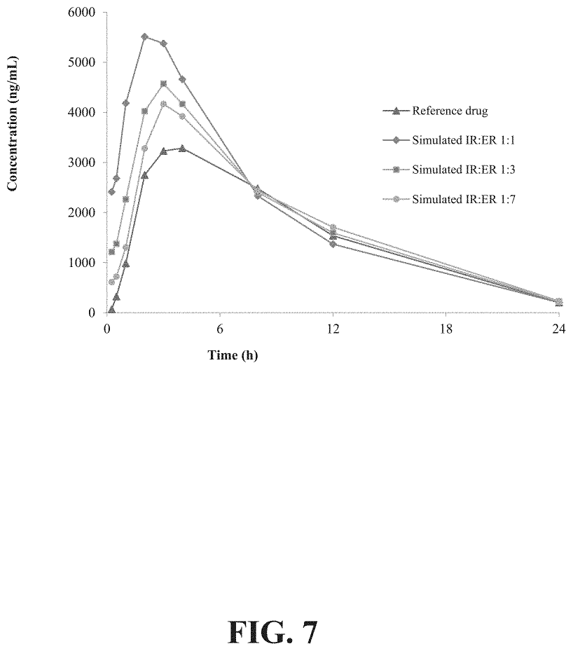

Using the PK curve information of the precursor drug dosage forms obtained in Example 1, the relative amounts of the drug in the IR portion and the ER portion necessary to achieve a desired composite PK profile of the oral drug dosage form were determined following the 3DPFbD® approach described herein. To achieve the desired composite PK profile, namely, a desired rapid initial pulse followed by a prolonged phase of drug delivery, different IR portions and ER portions were theoretically combined in variable ways and with different drug ratios such that the IR portion and ER portion could be assembled to achieve the designed oral drug dosage form comprising the IR portion and ER portion. The theoretical pharmacokinetic profiles of the oral drug dosage forms were simulated based on the predetermined pharmacokinetic curve of each component, and the formulation with a favorable outcome was selected for 3D printing and for further investigation in vivo. Based on the 3DPFbD® approach, it was determined that the desired theoretical PK profile of the composite oral drug dosage form could be achieved using an IR:ER drug ratio of 1:7. The simulated drug plasma concentration-time profiles of different IR:ER drug ratios, as compared to a reference ER drug dosage form having the same dose of the drug, are shown in FIG. 7.

### Example 3

Based on the simulated theoretical pharmacokinetics of the oral drug dosage form described in Example 2, the oral drug dosage form having a 1:7 drug ratio of the drug in the IR portion and ER portion was printed by 3D printing according to compositions and dimensions shown in Table 5 and Table 6.

TABLE 5
Composition of components of the oral drug dosage form.

| | Weight/tablet | | |
|---|---|---|---|
| Module | (mg) | Material | % |
| Immediate- | 20.8 | Drug | 60 |
| release | | Polyethylene glycol (PEG) 8000 | 35 |
| portion | | Croscarmellose sodium (CCNa) | 5 |
| Extended- | 291.7 | Drug | 30 |
| release | | Hydroxypropyl cellulose (HPC EF) | 50 |
| portion | | Polyethylene glycol (PEG) 400 | 20 |
| Shell | 250 | EUDRAGIT ® RS PO | 90 |
| | | Stearic acid | 10 |

TABLE 6
Dimensions of the components of the oral drug dosage form.

| Module | Radius (mm) | Inner Length (mm) | Thickness (mm) |
|---|---|---|---|
| Immediate-release | 3 | 12.5 | 0.2 |
| portion | | | |
| Extended-release | 3 | 12.5 | 3.0 |
| portion | | | |
| Shell | 3.6 | 12.5 | 4.0 |

In vivo pharmacokinetic studies were performed in fasted male beagle dogs. After oral administration of the oral drug dosage form or the reference drug dosage form, blood samples were collected from the jugular vein at predetermined times and the plasma concentration of drug was determined by LC-MS/MS analysis.

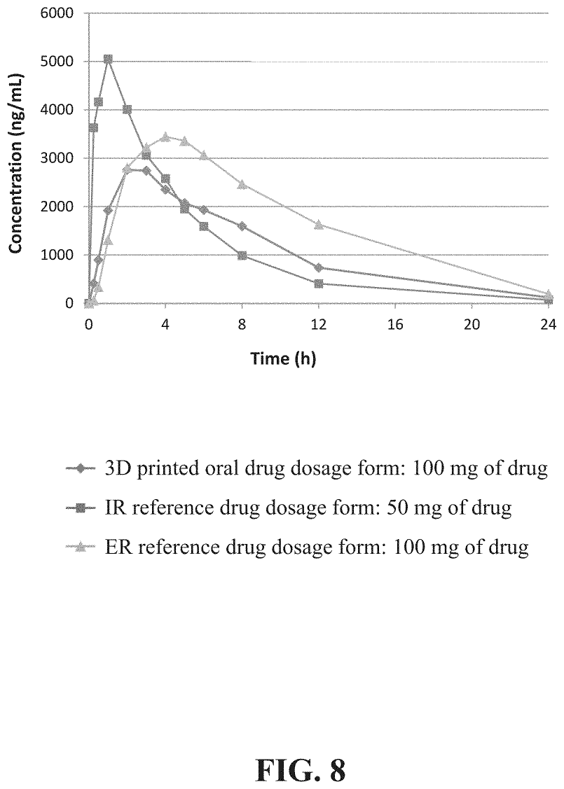

As can be seen from FIG. 8, the 3D-printed oral drug dosage form had a much smaller AUC and ˜2 hour earlier Tₘₐₓ when compared to the ER reference drug dosage form having the same dose of the drug (100 mg).

### Example 4

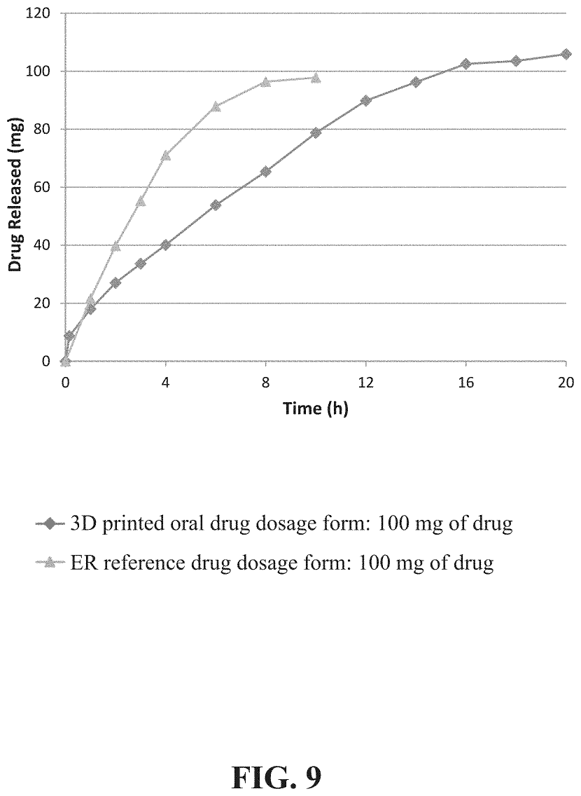

Optimization of the pharmacokinetics of the 3D-printed oral drug dosage form was performed. The dissolution rate of the 3D-printed oral drug dosage form and the ER reference drug dosage form was tested in vitro. As shown in FIG. 9, the 3D-printed oral drug dosage form had an in vitro dissolution rate of ˜16 hour while the ER reference drug dosage form had much faster in vitro dissolution rate (˜8 hours). In order to obtain a 3D-printed oral drug dosage form having an ER portion with an in vitro dissolution rate closer to that of the ER reference drug, the surface area and thickness of the ER portion was adjusted while keeping the same drug dose. A second ER precursor drug dosage form was printed according to the compositions and dimensions shown in Table 3 and Table 7. To increase the dissolution rate, the 3D-printed ER precursor dosage form was designed to have a larger surface area and smaller thickness as compared to the 3D-printed ER precursor dosage form in Example 1 (compare Table 4 and Table 7), but with the same amount of drug (87.5 mg of the drug).

TABLE 7
Dimensions of the components of the ER precursor dosage form.

| Module | Radius (mm) | Inner length (mm) | Thickness (mm) |
|---|---|---|---|
| Extended-release | 3.1 | 13 | 2.4 |
| portion | | | |
| Shell | 3.7 | 13 | 3.2 |

The 3D-printed ER precursor dosage form, having a larger surface area and smaller thickness, had a 12 hour in vitro dissolution rate (data not shown), which is faster than the in vitro dissolution of the 3D-printed ER precursor dosage form of Example 1 (˜16 hours).

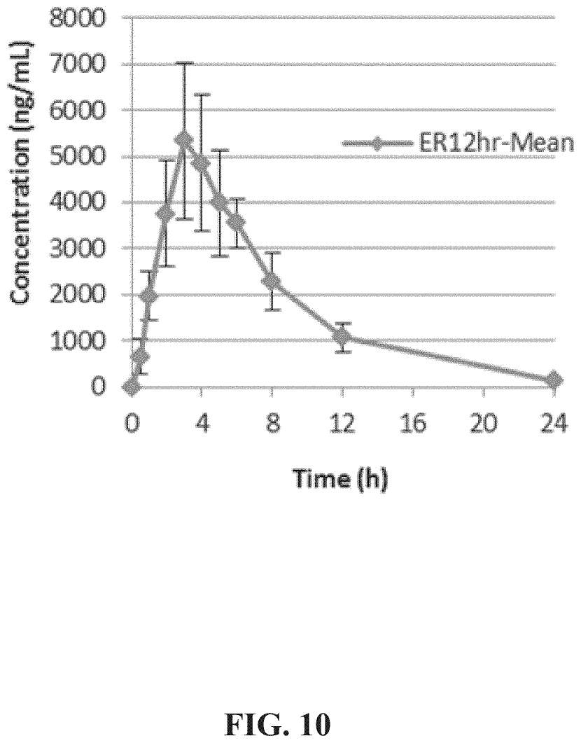

Using the optimized ER precursor dosage form configuration, an in vivo pharmacokinetic study was performed in fasted male beagle dogs. After oral administration of the 3D-printed ER precursor dosage form and a reference drug dosage form, blood samples were collected from jugular vein at predetermined times and the plasma concentration of the drug was determined by LC-MS/MS analysis. As can be seen from a comparison of FIGS. 6 and 10, the AUC of optimized ER precursor dosage form (FIG. 10) was larger than that of the ER precursor dosage form of Example 1 (FIG. 6), suggesting that the optimized ER precursor dosage form, which has an in vitro dissolution rate of 12 hours, is more desirable for use in the oral drug dosage form.

Using the same drug percentage in the ER material and the same surface area, an additional ER precursor dosage form was 3D printed having 100 mg of the drug to allow for a direct comparison of the 3D-printed ER precursor dosage form and the ER reference dosage form (100 mg of the drug). The 100 mg ER precursor dosage form was printed according to compositions and dimensions shown in Table 8 and Table 9. Using the 100 mg ER precursor dosage form and the 100 mg ER reference drug dosage form, an in vivo pharmacokinetic study was performed in fasted male beagle dogs. After oral administration of the 3D-printed ER precursor dosage form and the ER reference dosage form, blood samples were collected from the jugular vein at predetermined times and the plasma concentration of drug was determined by LC-MS/MS analysis.

TABLE 8
Composition of the components of the 100 mg ER
precursor dosage form.

| | Weight/ | | |
|---|---|---|---|
| | tablet | | |
| Module | (mg) | Material | % |
| Extended-release | 333.3 | Drug | 30 |
| portion | | Hydroxypropyl cellulose (HPC EF) | 50 |
| | | Polyethylene glycol (PEG) 400 | 20 |
| Shell | — | EUDRAGIT ® RS PO | 90 |
| | | Stearic acid | 10 |

TABLE 9
Dimensions of the components of the ER precursor dosage form.

| Module | Radius (mm) | Inner length (mm) | Thickness (mm) |
|---|---|---|---|
| Extended-release | 3.1 | 13 | 2.75 |
| portion | | | |
| Shell | 3.7 | 13 | 3.55 |

As can be seen from FIG. 11, replicates of in vivo pharmacokinetic profiles of the 3D-printed 100 mg ER precursor dosage form and the 100 mg ER reference drug dosage form are very similar, demonstrating overall similarity. The in vitro dissolution rate of the 3D-printed 100 mg ER precursor dosage form was about 12 hours (data not shown).

### Example 5

Theoretical pharmacokinetics of the oral drug dosage form comprising the optimized ER portion was simulated, according to Example 2, based on the PK curves of 3D-printed IR precursor dosage form from Example 1 and optimized 3D-printed ER precursor dosage form with 87.5 mg of drug from Example 4. The pharmacokinetics of the oral drug dosage form with IR:ER drug ratio of 1:7 demonstrated high similarity to the predicted pharmacokinetic profiles. Compared to the ER reference dosage form having same drug dose, the optimized 3D-printed oral drug dosage forms had a larger AUC, higher Cₘₐₓ, earlier Tₘₐₓ, and similar slowly descending plateau post-Cₘₐₓ.

Using the dimensions from the optimized ER precursor dosage form (87.5 mg of the drug in the ER portion), an oral drug dosage form was printed having an IR:ER drug ratio of 1:7 according to compositions and dimensions shown in Table 10 and Table 11.

An in vivo pharmacokinetic study was performed in fasted male beagle dogs. After oral administration of the 3D-printed oral drug dosage form and ER and IR reference drug dosage forms, blood samples were collected from the jugular vein at predetermined times and the plasma concentration of the drug was determined by LC-MS/MS analysis.

TABLE 10
Compositions of the components of the oral drug dosage form
(IR:ER = 1:7, 100 mg drug total).

| | Weight/tablet | | |
|---|---|---|---|
| Module | (mg) | Material | % |
| Immediate- | 20.8 | Drug | 60 |
| release | | Polyethylene glycol (PEG) 8000 | 35 |
| portion | | Croscarmellose sodium (CCNa) | 5 |
| Extended-release | 291.7 | Drug | 30 |
| portion | | Hydroxypropyl cellulose (HPC EF) | 50 |
| | | Polyethylene glycol (PEG) 400 | 20 |
| Shell | 250 | EUDRAGIT ® RS PO | 90 |
| | | Stearic acid | 10 |

TABLE 11
Dimensions of the components of the optimized oral
drug dosage form (IR:ER = 1:7).

| Module | Radius (mm) | Inner layer (mm) | Thickness (mm) |
|---|---|---|---|
| Immediate-release | 3.1 | 14.8 | 0.15 |
| portion | | | |
| Extended-release | 3.1 | 14.8 | 2.4 |
| portion | | | |
| Shell | 3.7 | 14.8 | 3.35 |

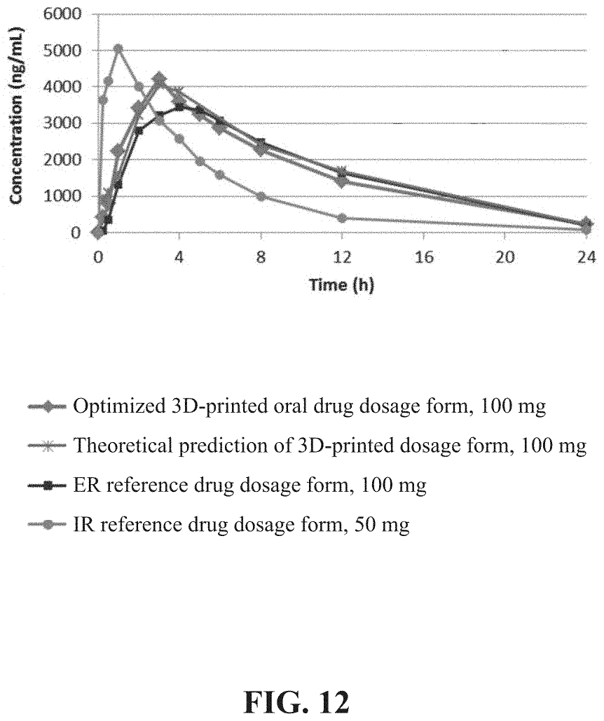

As can be seen from FIG. 12, the pharmacokinetics of the optimized 3D-printed oral drug dosage form were similar to the simulated theoretical pharmacokinetics of the oral drug dosage form. PK curves of IR reference drug dosage form (50 mg) and ER reference drug dosage form (100 mg) were also plotted as references. Compared to the same dose (100 mg) of the ER reference drug dosage form, the optimized 3D-printed oral drug dosage form showed a larger AUC, higher Cₘₐₓ, earlier Tₘₐₓ, and similar slowly descending plateau post-Cₘₐₓ; and the Cₘₐₓ of the optimized 3D-printed oral drug dosage form was lower than that of the IR reference drug dosage form.

Thus, the 3DPFbD® approach described herein was able to provide a customized, easy-to-adjust, and optimized 3D-printed oral drug dosage form with a desired PK profile having no dramatic fluctuation of plasma levels of the drug, faster drug effective plasma concentration, with longer and more stable plateau of effective drug plasma concentration, which would reduce side effect due to peak plasma levels of the drug when taken in high doses, and provide an easier administration regime, e.g., once-daily administration.

### Example 6

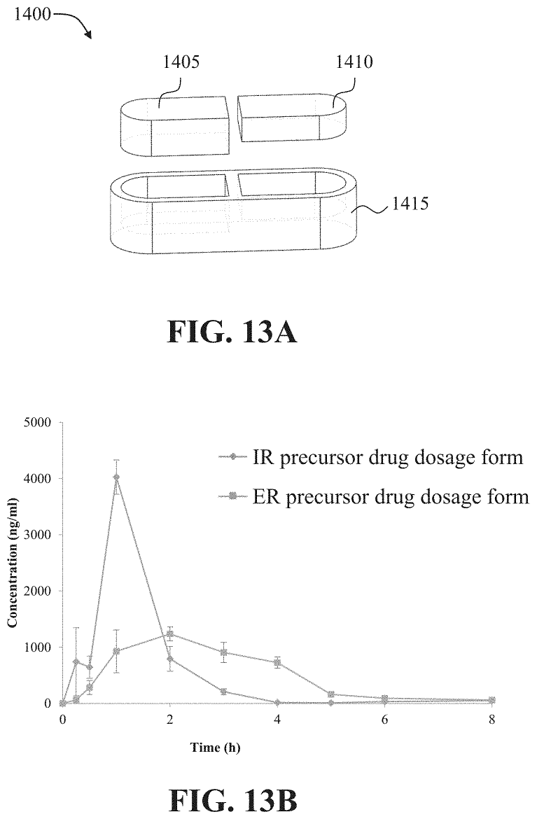

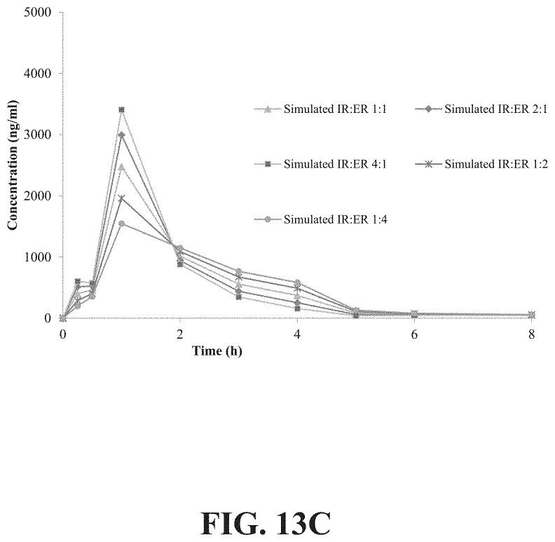

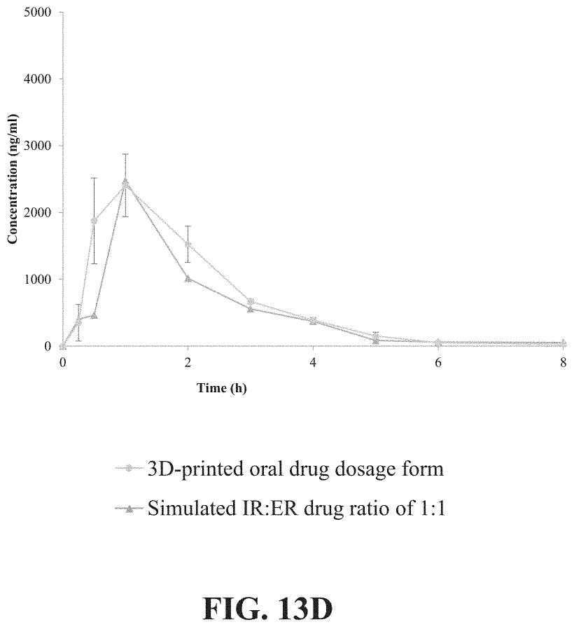

A BCS Class I drug (model drug) was incorporated into drug-containing portions of an oral drug dosage form comprising an IR portion and an ER portion positioned side-by-side and separated by a shell, the shell leaving the top surfaces of the IR portion and the ER portion exposed to fluid for concurrent release. A deconstructed view of the oral drug dosage form 1400 comprising an ER portion comprising the model drug 1405, an IR portion comprising the model drug 1410, and a shell 1415 is shown in FIG. 13A.

Using the designed oral drug dosage form, an IR precursor dosage form and an ER precursor dosage form were fabricated using a proprietary FDM pharmaceutical 3D printer. An in vivo pharmacokinetic study was performed in male beagle dogs fed low-fat diet to measure the PK curves of the precursor drug dosage forms. After oral administration of a 3D-printed IR precursor dosage form, a 3D-printed ER precursor dosage form, or a reference drug dosage form, blood samples were collected from the jugular vein at predetermined times and the plasma concentration of model drug was determined by LC-MS/MS analysis. The plasma concentrations following administration of the 3D-printed IR precursor dosage form and a 3D-printed ER precursor dosage form are shown in FIG. 13B.

Using the 3DPFbD® approach described herein, theoretical pharmacokinetics of oral drug dosage forms having different IR:ER drug ratios were simulated (FIG. 13C). An oral drug dosage form having an IR:ER drug ratio of 1:1 was selected to obtain the desired composite pharmacokinetic profile and the corresponding oral drug dosage form was 3D printed.

An in vivo pharmacokinetic study was performed in fasted male beagle dogs to measure the PK curve of the 3D-printed oral drug dosage form. After oral administration of the 3D-printed oral drug dosage form, blood samples were collected from the jugular vein at predetermined times and the plasma concentration of the drug was determined by LC-MS/MS analysis. As shown in FIG. 13D, the PK curve of the 3D-printed oral drug dosage form depicts modified release profile with a rapid initial peak followed by a smooth decline in plasma concentration, which was similar to the simulated theoretical pharmacokinetic curve.

1-71. (canceled)

72: A method of designing an oral drug dosage form having a fixed amount of a drug and a desired composite pharmacokinetic (PK) profile in an individual,
wherein the oral drug dosage form comprises a dosage unit comprising:
a first modulated-release (MR1) portion comprising the drug; and
a second modulated-release (MR2) portion comprising the drug,
the method comprising:
(a) obtaining a MR1 PK curve of a MR1 precursor drug dosage form comprising the MR1 portion in the individual;
(b) obtaining a MR2 PK curve of a MR2 precursor drug dosage form comprising the MR2 portion in the individual; and
(c) determining the relative amounts of the drug in the MR1 portion and the MR2 portion based on the MR1 PK curve and MR2 PK curve such that the MR1 portion and the MR2 portion when combined together produce the oral drug dosage form having the desired composite PK profile in the individual.

73: The method of claim 72, wherein the individual is a human.

74: The method of claim 72, wherein the drug has linear pharmacokinetics.

75: The method of claim 72, wherein the MR1 portion is a MR1 layer.

76: The method of claim 72, wherein the MR2 portion is a MR2 layer.

77: The method of claim 72, wherein the MR1 portion is an immediate-release (IR) portion having an immediate-release profile, and wherein the MR2 portion is an extended-release (ER) portion having an extended-release profile.

78: The method of claim 72, wherein the MR1 portion is a first extended-release (ER) portion having an extended-release profile, and wherein the MR2 portion is a second extended-release (ER) portion having an extended-release profile.

79: The method of claim 72, wherein the MR1 portion is a first immediate-release (IR) portion having an immediate-release profile, and wherein the MR2 portion is a second immediate-release (IR) portion having an immediate release profile.

80: The method of claim 77, wherein the MR1 portion and the MR2 portion are stacked on top of each other.

81: The method of claim 80, wherein the MR1 portion and MR2 portion are partially surrounded by a shell, and wherein the shell has a slower dissolution rate than the ER portion.

82: The method of claim 81, wherein the shell comprises an enteric material.

83: The method of claim 80, wherein the MR1 portion has a top surface and a bottom surface, wherein the MR2 portion has a top surface and a bottom surface, and wherein the shell is in direct contact with both the MR1 portion and the MR2 portion and leaves one surface of the MR1 portion and/or one surface of the MR2 portion exposed.

84: The method of claim 83, wherein the MR1 portion is stacked on top of the MR2 portion, and wherein the shell leaves only the top surface of the MR1 portion exposed.

85: The method of claim 77, wherein the oral drug dosage form further comprises a third modulated-release (MR3) portion, wherein the MR3 portion is an IR portion having an immediate-release profile or an ER portion having an extended-release profile.

86: The method of claim 85, wherein the shell separates the MR3 portion from the MR1 portion and the MR2 portion.

87: The method of claim 77, wherein the oral drug dosage form comprises two dosage units stacked back-to-back.

88: The method of claim 87, wherein the shell separates the two dosage units.

89: The method of claim 85, wherein the oral drug dosage form comprises two dosage units positioned side-by-side.

90: The method of claim 72, wherein at least 80% the MR1 portion erodes within about 60 minutes following administration of the oral drug dosage form to the individual.

91: The method of claim 90, wherein the MR1 portion comprises an erodible material.

92: The method of claim 72, wherein the MR2 portion comprises an erodible material, and wherein the drug contained in the MR2 portion is released from the oral drug dosage form over a period of at least about 5 hours.

93: The method of claim 72, wherein the desired composite PK profile is determined based on having an area under the curve (AUC) and Cₘₐₓ within an acceptable threshold of a reference PK curve of the drug.

94: The method of claim 93, wherein the desired composite PK profile is further determined based on having a tₘₐₓ within an acceptable threshold of a reference PK curve of the drug.

95: The method of claim 72, wherein the method comprises selecting one or more parameters for the MR2 portion to obtain a desired release profile of the drug from the MR2 portion.

96: The method of claim 95, wherein the one or more parameters is selected from the group consisting of: thickness, surface area, substrate erosion rate, and drug concentration in the MR2 portion.

97: The method of claim 72, further comprising determining the MR1 PK curve and the MR2 PK curve and adjusting the relative amounts of the drug in the MR1 portion and the MR2 portion.

98: The method of claim 72, further comprising determining a composite PK curve of the oral drug dosage form.

99: The method of claim 98, further comprising adjusting the relative amounts of the drug in the MR1 portion and the MR2 portion based on a comparison of the composite PK curve and the desired composite PK profile.

100: The method of claim 72, further comprising producing the oral drug dosage form by three-dimensional printing.

101: The method of claim 100, wherein the three-dimensional printing is carried out by melt extrusion deposition (MED).
